(19) United States
(12) Patent Application Publication
(10) Pub. No.: US 2020/0010482 A1
(43) Pub. Date: Jan. 9, 2020

(54) INHIBITORS OF BETA SECRETASE

(71) Applicant: Janssen Pharmaceutica NV, Beerse (BE)

(72) Inventors: Ann Marleen Vos, Boortmeerbeek (BE); Daniel Oehlrich, Geel (BE); Henricus Jacobus Maria Gijsen, Breda (NL); Karl Shawn Watts, Portland, OR (US); Sathesh Pangala Bhat, Jersey City, NJ (US); Frans Maria Alfons Van den Keybus, Essen (BE)

(21) Appl. No.: 16/489,885

(22) PCT Filed: Mar. 6, 2018
(86) PCT No.: PCT/EP2018/055401

§ 371 Date: Aug. 29, 2019

Related U.S. Application Data
(60) Provisional application No. 62/468,052, filed on Mar. 7, 2017.

(30) Foreign Application Priority Data
Sep. 7, 2017 (EP) 17189762.2

(51) Int. Cl. C07D 513/04; C07D 498/04; A61P 25/28

(52) U.S. Cl. CPC C07D 513/04; A61P 25/28; C07D 498/04

(57) ABSTRACT

The present invention relates to tricyclic inhibitors of beta-secretase having the structure shown in Formula (I) and (II)

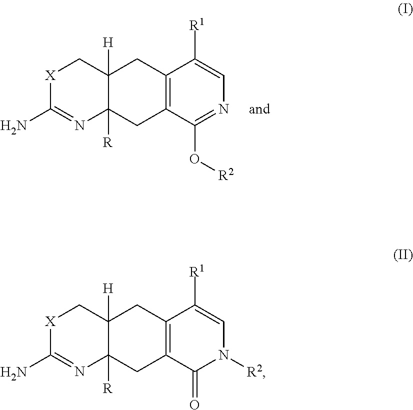

and the tautomers and the stereoisomeric forms thereof, wherein the radicals are as defined in the specification. The invention is also directed to pharmaceutical compositions comprising such compounds, to processes for preparing such compounds and compositions, and to the use of such compounds and compositions for the prevention and treatment of disorders in which beta-secretase is involved, such as Alzheimer's disease (AD), mild cognitive impairment, senility, dementia, dementia with Lewy bodies, Down's syndrome, dementia associated with stroke, dementia associated with Parkinson's disease, dementia associated with beta-amyloid, age-related macular degeneration, type 2 diabetes and other metabolic disorders.

### FIELD OF THE INVENTION

The present invention relates to tricyclic inhibitors of beta-secretase having the structure shown in Formula (I) and (II)

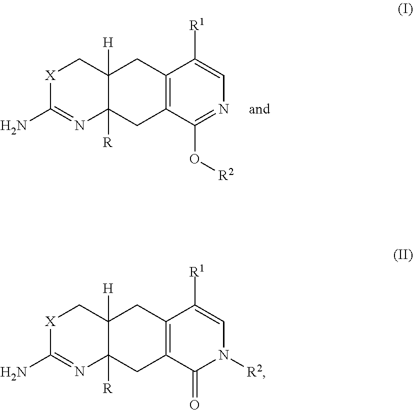

and the tautomers and the stereoisomeric forms thereof, wherein the radicals are as defined in the specification. The invention is also directed to pharmaceutical compositions comprising such compounds, to processes for preparing such compounds and compositions, and to the use of such compounds and compositions for the prevention and treatment of disorders in which beta-secretase is involved, such as Alzheimer's disease (AD), mild cognitive impairment, senility, dementia, dementia with Lewy bodies, Down's syndrome, dementia associated with stroke, dementia associated with Parkinson's disease, dementia associated with beta-amyloid, age-related macular degeneration, type 2 diabetes and other metabolic disorders.

### BACKGROUND OF THE INVENTION

Alzheimer's Disease (AD) is a neurodegenerative disease associated with aging. AD patients suffer from cognition deficits and memory loss as well as behavioral problems such as anxiety. Over 90% of those afflicted with AD have a sporadic form of the disorder while less than 10% of the cases are familial or hereditary. In the United States, about one in ten people at age 65 have AD while at age 85, one out of every two individuals are afflicted by AD. The average life expectancy from the initial diagnosis is 7-10 years, and AD patients require extensive care either in an assisted living facility or by family members. With the increasing number of elderly in the population, AD is a growing medical concern. Currently available therapies for AD merely treat the symptoms of the disease and include acetylcholinesterase inhibitors to improve cognitive properties as well as anxiolytics and antipsychotics to control the behavioral problems associated with this ailment.

The hallmark pathological features in the brain of AD patients are neurofibrillary tangles which are generated by hyperphosphorylation of tau protein and amyloid plaques which form by aggregation of beta-amyloid 1-42 (Abeta 1-42) peptide. Abeta 1-42 forms oligomers and then fibrils, and ultimately amyloid plaques. The oligomers and fibrils are believed to be especially neurotoxic and may cause most of the neurological damage associated with AD. Agents that prevent the formation of Abeta 1-42 have the potential to be disease-modifying agents for the treatment of AD. Abeta 1-42 is generated from the amyloid precursor protein (APP), comprised of 770 amino acids. The N-terminus of Abeta 1-42 is cleaved by beta-secretase (BACE1), and then gamma-secretase cleaves the C-terminal end. In addition to Abeta 1-42, gamma-secretase also liberates Abeta 1-40 which is the predominant cleavage product as well as Abeta 1-38 and Abeta 1-43. These Abeta forms can also aggregate to form oligomers and fibrils. Thus, inhibitors of BACE1 would be expected to prevent the formation of Abeta 1-42 as well as Abeta 1-40, Abeta 1-38 and Abeta 1-43 and would be potential therapeutic agents in the treatment of AD.

Type 2 diabetes (T2D) is caused by insulin resistance and inadequate insulin secretion from pancreatic beta-cells leading to poor blood-glucose control and hyperglycemia. Patients with T2D have an increased risk of microvascular and macrovascular disease and a range of related complications including diabetic nephropathy, retinopathy and cardiovascular disease. The rise in prevalence of T2D is associated with an increasingly sedentary lifestyle and high-energy food intake of the world's population.

Beta-cell failure and consequent dramatic decline in insulin secretion and hyperglycemia marks the onset of T2D. Most current treatments do not prevent the loss of beta-cell mass characterizing overt T2D. However, recent developments with GLP-1 analogues, gastrin and other agents show that preservation and proliferation of beta-cells is possible to achieve, leading to an improved glucose tolerance and slower progression to overt T2D.

Tmem27 has been identified as a protein promoting beta-cell proliferation and insulin secretion. Tmem27 is a 42 kDa membrane glycoprotein which is constitutively shed from the surface of beta-cells, resulting from a degradation of the full-length cellular Tmem27. Overexpression of Tmem27 in a transgenic mouse increases beta-cell mass and improves glucose tolerance in a diet-induced obesity DIO model of diabetes. Furthermore, siRNA knockout of Tmem27 in a rodent beta-cell proliferation assay (e.g. using INS1e cells) reduces the proliferation rate, indicating a role for Tmem27 in control of beta-cell mass.

BACE2 is the protease responsible for the degradation of Tmem27. It is a membrane-bound aspartyl protease and is co-localized with Tmem27 in human pancreatic beta-cells. It is also known to be capable of degrading APP, IL-1R2 and ACE2. The capability to degrade ACE2 indicates a possible role of BACE2 in the control of hypertension.

Inhibitors of BACE1 and/or BACE2 can in addition be used for the therapeutic and/or prophylactic treatment of amyotrophic lateral sclerosis (ALS), arterial thrombosis, autoimmune/inflammatory diseases, cancer such as breast cancer, cardiovascular diseases such as myocardial infarction and stroke, dermatomyositis, Down's Syndrome, gastrointestinal diseases, Glioblastoma multiforme, Graves Disease, Huntington's Disease, inclusion body myositis (IBM), inflammatory reactions, Kaposi Sarcoma, Kostmann Disease, lupus erythematosus, macrophagic myofasciitis, juvenile idiopathic arthritis, granulomatous arthritis, malignant melanoma, multiple myeloma, rheumatoid arthritis, Sjogren syndrome, SpinoCerebellar Ataxia 1, SpinoCerebellar Ataxia 7, Whipple's Disease or Wilson's Disease.

### SUMMARY OF THE INVENTION

The present invention is directed to compounds of Formula (I) and (II)

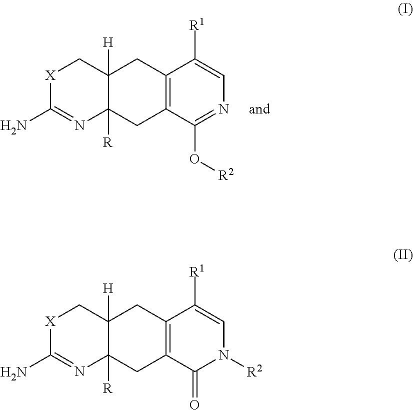

and the tautomers and the stereoisomeric forms thereof, wherein

X is S or O;

R is phenyl optionally substituted with 1, 2 or 3 substituents each independently selected from the group consisting of halo; hydroxyl; C₁₋₃alkyl; cyano; nitro; Het; Ar; (C₁₋₃alkyloxy)C₁₋₃alkyl-NH—C₁₋₃alkyl-; C₁₋₆alkyloxy optionally substituted with cyano, or C₁₋₃alkyloxy; C₂₋₆alkynyloxy; tetrahydro-2H-pyranyloxy; Ar-oxy-; Het-oxy-; Ar—CH(OH)—; —NR^{a}R^{b}; a divalent —NH—CH₂CH₂—O— substituent optionally substituted with 1 or 2 substituents each independently selected from halo and oxo; C₁₋₄alkyl(C═O)—; Ar(C═O)—; and R³—C₁₋₆alkyloxy-; wherein

Het is selected from pyridinyl and pyrimidinyl, each of which can be optionally substituted with halo, cyano, C₁₋₃alkyl, C₁₋₃alkyloxy, —CF₃, and —OCF₃;
Ar is phenyl optionally substituted with halo, cyano, C₁₋₃alkyl, C₁₋₃alkyloxy, —CF₃, —OCF₃;
R^{a} is selected from H, or C₁₋₃alkyl; and
R^{b} is selected from C₁₋₃alkyl, (C₁₋₃alkyloxy)C₁₋₃alkyl(C═O)—, or Het¹(C═O)—;
R³ is selected from the group consisting of C₃₋₆cycloalkyl; Het¹; Ar¹; tetrahydro-2H-pyranyl; C₃₋₆cycloalkyloxy; tetrahydro-2H-pyranyloxy; Het¹-oxy-; and Ar¹-oxy-; wherein
Ar¹ is phenyl optionally substituted with halo, cyano, C₁₋₃alkyl, C₁₋₃alkyloxy, cyano-C₁₋₃alkyloxy —CF₃, or —OCF₃;
Het¹ is selected from the group consisting of pyridyl, pyrimidinyl, pyrazinyl, pyridazinyl, thienyl, pyrazolyl, isoxazolyl, 1H-imidazolyl, thiazolyl, oxazolyl, 1H-indolyl, and 1H-indazolyl; each of which is optionally substituted with 1 or 2 substituents each independently selected from the group consisting of halo, cyano,
C₁₋₃alkyl, C₁₋₃alkyloxy, —CF₃, and —OCF₃;

R¹ is selected from the group consisting of hydrogen; halo; cyano; C₁₋₃alkyl optionally substituted with hydroxyl or C₁₋₃alkyloxy; C₃₋₆cycloalkyl; C₃₋₆cycloalkenyl; (C₃₋₆cycloalkyl)C₁₋₃alkyl; C₁₋₃alkyloxy; —NR^{x}R^{y}; C₁₋₃alkyloxy-(C═O)—; C₁₋₃alkyloxy-C₂₋₃alkenyl; (halo-phenyl)-C₂₋₃alkenyl-; heterocyclyl; homoaryl; heteroaryl;

C₃₋₆cycloalkyloxy; homoaryloxy; heteroaryloxy; homoaryl-CH₂-oxy; and heteroaryl-CH₂-oxy; wherein
R^{x} is hydrogen or C₁₋₃alkyl;
R^{y} is C₁₋₃alkyl or phenyl optionally substituted 1, 2, or 3 substituents each independently selected from halo, C₁₋₃alkyl, and C₁₋₃alkyloxy;
heterocyclyl is selected from the group consisting of piperidinyl, morpholinyl, 3,4-dihydro-2H-pyranyl; and tetrahydro-2H-pyranyl, each of which being optionally substituted with 1, 2 or 3 substituents each independently selected from the group consisting of C₁₋₃alkyl, C₃₋₆cycloalkyl and oxo;
homoaryl is phenyl optionally substituted with 1, 2 or 3 substituents each independently selected from the group consisting of halo, hydroxyl, cyano, C₁₋₃alkyl, mono-halo-C₁₋₃alkyl, poly-halo-C₁₋₃alkyl, cyano-C₁₋₃alkyl, C₁₋₃alkyloxy, mono-halo-C₁₋₃alkyloxy, poly-halo-C₁₋₃alkyloxy, C₁₋₃alkyloxy-(C═O)—, phenyloxy-, NR^{1a}R^{1b}, —(C═O)NR^{1a}R^{1b}, 1H-pyrazolyl optionally substituted with 1 or 2 methyl substituents; or is naphthalenyl, optionally substituted with C₁₋₃alkyl or C₁₋₃alkyloxy; wherein R^{1a} is hydrogen or C₁₋₃alkyl and R^{1b} is C₁₋₃alkyl, or NR^{1a}R^{1b} form together a 1-pyrrolidinyl, 1-piperidinyl, 4-piperazinyl or a 4-morpholinyl;
heteroaryl is selected from the group consisting of pyridyl, 2-oxo-1,2-dihydropyridinyl, 6-oxo-1,6-dihydropyridinyl, pyrimidinyl, pyrazinyl, pyridazinyl, pyrazolyl, isoxazolyl, oxazolyl, thiophenyl, indolyl, indazolyl, 1-benzothienyl, 1-benzofuranyl, isoquinolinyl, 5,6,7,8-tetrahydroquinolinyl, 3,4-dihydro-2H-chromenyl and 3,4-dihydro-2H-pyrido[3,2-b][1,4]oxazinyl, each of which is optionally substituted with 1, 2 or 3 substituents each independently selected from the group consisting of halo, cyano,
C₁₋₃alkyl, mono-haloC₁₋₃alkyl, poly-halo-C₁₋₃alkyl, C₁₋₃alkyloxy, C₃₋₆cycloalkyl, tetrahydro-2H-pyranyl, phenyl optionally substituted with C₁₋₃alkyl, and —NR^{1c}R^{1d}; wherein
R^{1c} is hydrogen or C₁₋₃alkyl, R^{1d} is C₁₋₃alkyl, or NR^{1c}R^{1d} form together 1-pyrrolidinyl, 1-piperidinyl, 4-piperazinyl, 4-morpholinyl or 1H-imidazolyl, each of which is optionally substituted with C₁₋₃alkyl; and

R² is hydrogen or C₁₋₃alkyl;

and the pharmaceutically acceptable addition salts and the solvates thereof.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

Exemplifying the invention are methods of treating a disorder mediated by the beta-secretase enzyme, comprising administering to a subject in need thereof a therapeutically effective amount of any of the compounds or pharmaceutical compositions described above.

Further exemplifying the invention are methods of inhibiting the beta-secretase enzyme, comprising administering to a subject in need thereof a therapeutically effective amount of any of the compounds or pharmaceutical compositions described above.

An example of the invention is a method of treating a disorder selected from the group consisting of Alzheimer's disease, mild cognitive impairment, senility, dementia, dementia with Lewy bodies, Down's syndrome, dementia associated with stroke, dementia associated with Parkinson's disease, dementia associated with beta-amyloid, and age-related macular degeneration, preferably Alzheimer's disease, type 2 diabetes and other metabolic disorders, comprising administering to a subject in need thereof, a therapeutically effective amount of any of the compounds or pharmaceutical compositions described above.

Another example of the invention is any of the compounds described above for use in treating: (a) Alzheimer's Disease, (b) mild cognitive impairment, (c) senility, (d) dementia, (e) dementia with Lewy bodies, (f) Down's syndrome, (g) dementia associated with stroke, (h) dementia associated with Parkinson's disease, (i) dementia associated with beta-amyloid or (j) age-related macular degeneration, (k) type 2 diabetes and (1) other metabolic disorders in a subject in need thereof.

### DETAILED DESCRIPTION OF THE INVENTION

The present invention is directed to compounds of formula (I) as defined hereinbefore, and pharmaceutically acceptable addition salts and solvates thereof. The compounds of formula (I) are inhibitors of the beta-secretase enzyme (also known as beta-site cleaving enzyme, BACE, BACE1, Asp2 or memapsin 2, or BACE2), and may be useful in the treatment of Alzheimer's disease, mild cognitive impairment, senility, dementia, dementia associated with stroke, dementia with Lewy bodies, Down's syndrome, dementia associated with Parkinson's disease, dementia associated with beta-amyloid, and age-related macular degeneration, preferably Alzheimer's disease, mild cognitive impairment or dementia, more preferably Alzheimer's disease, type 2 diabetes and other metabolic disorders.

In an embodiment the invention relates to compounds of Formula (I) and (III) as described herein, wherein

X is S or O;

R is phenyl optionally substituted with 1, 2 or 3 substituents each independently selected from the group consisting of halo; hydroxyl; C₁₋₃alkyl; cyano; nitro; Het; Ar; (C₁₋₃alkyloxy)C₁₋₃alkyl-NH—C₁₋₃alkyl-; C₁₋₆alkyloxy optionally substituted with cyano, or C₁₋₃alkyloxy; C₂₋₆alkynyloxy; tetrahydro-2H-pyranyloxy; Ar-oxy-; Het-oxy-; —NR^{a}R^{b}; a divalent —NH—CH₂CH₂—O— substituent optionally substituted with 1 or 2 substituents each independently selected from halo and oxo; and R³—C₁₋₆alkyloxy-; wherein

Het is selected from pyridinyl and pyrimidinyl, each of which can be optionally substituted with cyano;
Ar is phenyl;
R^{a} is selected from H, or C₁₋₃alkyl; and
R^{b} is selected from C₁₋₃alkyl, and (C₁₋₃alkyloxy)C₁₋₃alkyl(C═O)—;
R³ is selected from the group consisting of C₃₋₆cycloalkyl; Het¹; Ar¹; tetrahydro-2H-pyranyl; C₃₋₆cycloalkyloxy; tetrahydro-2H-pyranyloxy; Het¹-oxy-; and Ar¹-oxy-; wherein
Ar¹ is phenyl optionally substituted with halo, cyano, C₁₋₃alkyl, and C₁₋₃alkyloxy;
Het¹ is selected from the group consisting of pyridyl, pyrimidinyl, pyrazinyl, pyridazinyl, thienyl, pyrazolyl, isoxazolyl, 1H-imidazolyl, thiazolyl, oxazolyl, 1H-indolyl, and 1H-indazolyl; each of which is optionally substituted with 1 or 2 substituents each independently selected from the group consisting of halo, cyano, and C₁₋₃alkyl;
R¹ is selected from the group consisting of hydrogen; halo; cyano; C₁₋₃alkyl optionally substituted with hydroxyl or C₁₋₃alkyloxy; C₃₋₆cycloalkyl; C₁₋₃alkyloxy; C₁₋₃alkyloxy-(C═O)—; C₁₋₃alkyloxyC₂₋₃alkenyl; (halo-phenyl)-C₂₋₃alkenyl-; heterocyclyl; homoaryl; heteroaryl; homoaryl-CH₂-oxy; and heteroaryl-CH₂-oxy; wherein
heterocyclyl is 3,4-dihydro-2H-pyranyl;
homoaryl is phenyl optionally substituted with 1, 2 or 3 substituents each independently selected from the group consisting of halo, hydroxyl, cyano, C₁₋₃alkyl, mono-halo-C₁₋₃alkyl, poly-halo-C₁₋₃alkyl, cyano-C₁₋₃alkyl, C₁₋₃alkyloxy, mono-halo-C₁₋₃alkyloxy, poly-halo-C₁₋₃alkyloxy, C₁₋₃alkyloxy-(C═O)—, phenyloxy-, NR^{1a}R^{1b}, and —(C═O)NR^{1a}R^{1b};
or is naphthalenyl, optionally substituted with C₁₋₃alkyl or
C₁₋₃alkyloxy; wherein R^{1a} is hydrogen or C₁₋₃alkyl and R^{1b} is C₁₋₃alkyl, or NR^{1a}R^{1b} form together a 4-morpholinyl;
heteroaryl is selected from the group consisting of pyridyl, 2-oxo-1,2-dihydropyridinyl, 6-oxo-1,6-dihydropyridinyl, pyrimidinyl, pyrazinyl, pyridazinyl, pyrazolyl, isoxazolyl, oxazolyl, thiophenyl, indolyl, indazolyl, 1-benzothienyl, 1-benzofuranyl, isoquinolinyl, 5,6,7,8-tetrahydroquinolinyl, 3,4-dihydro-2H-chromenyl and 3,4-dihydro-2H-pyrido[3,2-b][1,4]oxazinyl, each of which is optionally substituted with 1, 2 or 3 substituents each independently selected from the group consisting of halo, cyano,
C₁₋₃alkyl, mono-haloC₁₋₃alkyl, poly-halo-C₁₋₃alkyl, C₁₋₃alkyloxy, tetrahydro-2H-pyranyl, phenyl optionally substituted with C₁₋₃alkyl, and —NR^{1c}R^{1d}; wherein
R^{1c} is hydrogen or C₁₋₃alkyl, R^{1d} is C₁₋₃alkyl, or NR^{1c}R^{1d} form together 1-pyrrolidinyl, 1-piperidinyl, 4-piperazinyl, 4-morpholinyl or 1H-imidazolyl, each of which is optionally substituted with C₁₋₃alkyl; and
R² is hydrogen or C₁₋₃alkyl.

In another embodiment, the invention relates to compounds of Formula (I) and (II) as described herein, wherein

X is S or O;

R is phenyl optionally substituted with 1, 2 or 3 substituents each independently selected from the group consisting of halo; hydroxyl; nitro; Het; C₁₋₆alkyloxy optionally substituted with cyano, or C₁₋₃alkyloxy; C₂₋₆alkynyloxy; tetrahydro-2H-pyranyloxy; Het-oxy-; —NR^{a}R^{b}; a divalent —NH—CH₂CH₂—O— substituent optionally substituted with 1 or 2 substituents each independently selected from halo and oxo; and R³—C₁₋₆alkyloxy-; wherein

Het is selected from pyridinyl and pyrimidinyl, each of which can be optionally substituted with cyano;
Ar is phenyl;

### R^{a} is H; and

R^{b} is (C₁₋₃alkyloxy)C₁₋₃alkyl(C═O)—;
R³ is selected from the group consisting of C₃₋₆cycloalkyl; Het¹; Ar¹; tetrahydro-2H-pyranyl; C₃₋₆cycloalkyloxy; tetrahydro-2H-pyranyloxy; Het¹-oxy-; and Ar¹-oxy-; wherein
Ar¹ is phenyl optionally substituted with halo, cyano, C₁₋₃alkyl, and C₁₋₃alkyloxy;
Het¹ is selected from the group consisting of pyridyl, pyrimidinyl, isoxazolyl, 1H-imidazolyl, thiazolyl, and 1H-indazolyl; each of which is optionally substituted with 1 or 2 substituents each independently selected from C₁₋₃alkyl;
R¹ is selected from the group consisting of hydrogen; halo; cyano; C₁₋₃alkyl optionally substituted with hydroxyl or C₁₋₃alkyloxy; C₃₋₆cycloalkyl; C₁₋₃alkyloxy; C₁₋₃alkyloxy-(C═O)—; C₁₋₃alkyloxyC₂₋₃alkenyl; (halo-phenyl)-C₂₋₃alkenyl-; heterocyclyl; homoaryl; heteroaryl; homoaryl-CH₂-oxy; and heteroaryl-CH₂-oxy; wherein
heterocyclyl is 3,4-dihydro-2H-pyranyl;
homoaryl is phenyl optionally substituted with 1, 2 or 3 substituents each independently selected from the group consisting of halo, hydroxyl, cyano, C₁₋₃alkyl, mono-halo-C₁₋₃alkyl, poly-halo-C₁₋₃alkyl, cyano-C₁₋₃alkyl, C₁₋₃alkyloxy, mono-halo-C₁₋₃alkyloxy, poly-halo-C₁₋₃alkyloxy, C₁₋₃alkyloxy-(C═O)—, phenyloxy-, NR^{1a}R^{1b}, and —(C═O)NR^{1a}R^{1b}; or is naphthalenyl, optionally substituted with C₁₋₃alkyl or
C₁₋₃alkyloxy; wherein R^{1a} is hydrogen or C₁₋₃alkyl and R^{1b} is C₁₋₃alkyl, or NR^{1a}R^{1b} form together a 4-morpholinyl;
heteroaryl is selected from the group consisting of pyridyl, 2-oxo-1,2-dihydropyridinyl, 6-oxo-1,6-dihydropyridinyl, pyrimidinyl, pyrazinyl, pyridazinyl, pyrazolyl, isoxazolyl, oxazolyl, thiophenyl, indolyl, indazolyl, 1-benzothienyl, 1-benzofuranyl, isoquinolinyl, 5,6,7,8-tetrahydroquinolinyl, 3,4-dihydro-2H-chromenyl and 3,4-dihydro-2H-pyrido[3,2-b][1,4]oxazinyl, each of which is optionally substituted with 1, 2 or 3 substituents each independently selected from the group consisting of halo, cyano,
C₁₋₃alkyl, mono-haloC₁₋₃alkyl, poly-halo-C₁₋₃alkyl, C₁₋₃alkyloxy, tetrahydro-2H-pyranyl, phenyl optionally substituted with C₁₋₃alkyl, and —NR^{1c}R^{1d}; wherein
R^{1c} is hydrogen or C₁₋₃alkyl, R^{1d} is C₁₋₃alkyl, or NR^{1c}R^{1d} form together 1-pyrrolidinyl, 4-piperazinyl, or 1H-imidazolyl, each of which is optionally substituted with C₁₋₃alkyl; and
R² is hydrogen or C₁₋₃alkyl.

In a further embodiment, the invention relates to compounds of Formula (I) and (II) as described herein, wherein

R is phenyl optionally substituted with 1, 2 or 3 substituents each independently selected from the group consisting of halo; and Het; wherein

Het is selected from pyridinyl and pyrimidinyl, each of which can be optionally substituted with cyano.

In another embodiment, the invention relates to compounds of Formula (I) and (II) as described herein, wherein

X is S or O;

R is phenyl optionally substituted with 1, 2 or 3 substituents each independently selected from the group consisting of halo; C₁₋₆alkyloxy optionally substituted with cyano, or C₁₋₃alkyloxy; C₂₋₆alkynyloxy; tetrahydro-2H-pyranyloxy; and R³—C₁₋₆alkyloxy-; wherein

R³ is selected from the group consisting of C₃₋₆cycloalkyl; Ar¹; tetrahydro-2H-pyranyl; C₃₋₆cycloalkyloxy; tetrahydro-2H-pyranyloxy; Het¹-oxy-; and Ar¹-oxy-; wherein
Ar¹ is phenyl optionally substituted with halo, cyano, C₁₋₃alkyl, and C₁₋₃alkyloxy;
Het¹ is selected from the group consisting of pyridyl, pyrimidinyl, isoxazolyl, 1H-imidazolyl, thiazolyl, and 1H-indazolyl; each of which is optionally substituted with 1 or 2 substituents each independently selected from C₁₋₃alkyl;
R¹ is selected from the group consisting of hydrogen; halo; homoaryl; and heteroaryl; wherein
homoaryl is phenyl optionally substituted with 1 or 2 or 3 substituents each independently selected from the group consisting of halo, cyano, C₁₋₃alkyl, and C₁₋₃alkyloxy;
heteroaryl is selected from the group consisting of pyridyl and isoxazolyl, each of which is optionally substituted with 1 or 2 substituents each independently selected from the group consisting of halo, C₁₋₃alkyl and C₁₋₃alkyloxy; and
R² is hydrogen or C₁₋₃alkyl.

In another embodiment, the invention relates to compounds of Formula (I) and (II) as described herein, wherein

X is S or O;

R¹ is homoaryl or heteroaryl; wherein
homoaryl is phenyl optionally substituted with 1 or 2 substituents each independently selected from the group consisting of halo, cyano, and C₁₋₃alkyl;
heteroaryl is selected from the group consisting of pyridyl, and isoxazolyl, each of which is optionally substituted with 1 or 2 substituents each independently selected from the group consisting of halo, cyano, and C₁₋₃alkyl; and
R² is hydrogen or C₁₋₃alkyl.

The invention relates in particular to compounds wherein carbon centres C₄ₐ and C₁₀ₐ in the tricyclic scaffold are of cis configuration (i.e. H and R are projected towards the same side out of the plane of the scaffold)

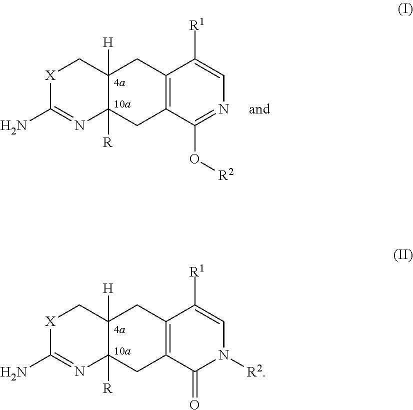

Thus, in particular, the invention relates to compounds of Formula (I′) and (II″) and compounds of Formula (I′) and (II″) as represented below, wherein the tricyclic core is in the plane of the drawing and H and R are projected above the plane of the drawing (with the bond shown with a bold wedge [symbol] ) in (I′) and (II′) or wherein the tricyclic core is in the plane of the drawing and H and R are projected below the plane of the drawing (with the bond shown with a wedge of parallel lines [symbol] ):

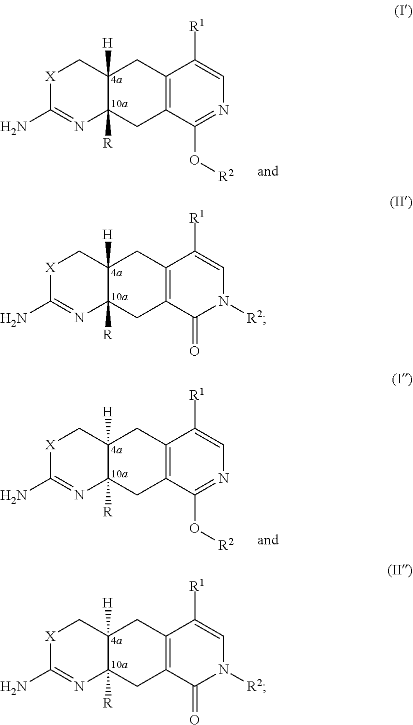

### Definitions

“Halo” shall denote fluoro, chloro and bromo; “C₁₋₃alkyl” and “C₁₋₆alkyl” shall denote a straight or branched saturated alkyl group having 1, 2 or 3 carbon atoms or 1, 2, 3, 4, 5, or 6 carbon atoms, respectively e.g. methyl, ethyl, 1-propyl, 2-propyl, etc.; “C₁₋₃alkyloxy” shall denote an ether radical wherein C₁₋₃alkyl is as defined before; “mono- and polyhaloC₁₋₃alkyl” shall denote C₁₋₃alkyl as defined before, substituted with 1, 2, 3 or where possible with more halo atoms as defined before; “mono- and polyhalo-C₁₋₃alkyloxy” shall denote an ether radical wherein mono- and polyhaloC₁₋₃alkyl is as defined before; “C₃₋₆cycloalkyl” shall denote cyclopropyl, cyclobutyl, cyclopentyl and cyclohexyl; “C₃₋₆cycloalkenyl” shall denote a C₃₋₆cycloalkyl radical bearing a C═C bond; “C₂₋₆alkynyl” shall denote a straight or branched acyclic group having 2 to 6 carbon atoms wherein at least one carbon-carbon bond is a triple bond; “C₂₋₃alkenyl” shall denote a straight or branched acyclic group having 2 to 3 carbon atoms wherein a carbon-carbon bond is a double bond.

The term “subject” as used herein, refers to an animal, preferably a mammal, most preferably a human, who is or has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

Hereinbefore and hereinafter, the term “compound of formula (I)” is meant to include the addition salts, the solvates and the stereoisomers thereof.

The terms “stereoisomers” or “stereochemically isomeric forms” hereinbefore or hereinafter are used interchangeably.

The invention includes all stereoisomers of the compound of Formula (I) either as a pure stereoisomer or as a mixture of two or more stereoisomers.

Enantiomers are stereoisomers that are non-superimposable mirror images of each other. A 1:1 mixture of a pair of enantiomers is a racemate or racemic mixture. Diastereomers (or diastereoisomers) are stereoisomers that are not enantiomers, i.e. they are not related as mirror images. If a compound contains a double bond, the substituents may be in the E or the Z configuration. If a compound contains a disubstituted cycloalkyl group, the substituents may be in the cis or trans configuration. Therefore, the invention includes enantiomers, diastereomers, racemates, E isomers, Z isomers, cis isomers, trans isomers and mixtures thereof.

The absolute configuration is specified according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of formula (I) is for instance specified as (R), this means that the compound is substantially free of the (S) isomer; when a compound of formula (I) is for instance specified as E, this means that the compound is substantially free of the Z isomer; when a compound of formula (I) is for instance specified as cis, this means that the compound is substantially free of the trans isomer.

For use in medicine, the addition salts of the compounds of this invention refer to non-toxic “pharmaceutically acceptable addition salts”. Other salts may, however, be useful in the preparation of compounds according to this invention or of their pharmaceutically acceptable addition salts. Suitable pharmaceutically acceptable addition salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable addition salts thereof may include alkali metal salts, e.g., sodium or potassium salts; alkaline earth metal salts, e.g., calcium or magnesium salts; and salts formed with suitable organic ligands, e.g., quaternary ammonium salts.

Representative acids which may be used in the preparation of pharmaceutically acceptable addition salts include, but are not limited to, the following: acetic acid, 2,2-dichloroactic acid, acylated amino acids, adipic acid, alginic acid, ascorbic acid,

L-aspartic acid, benzenesulfonic acid, benzoic acid, 4-acetamidobenzoic acid, (+)-camphoric acid, camphorsulfonic acid, capric acid, caproic acid, caprylic acid, cinnamic acid, citric acid, cyclamic acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, beta-oxo-glutaric acid, glycolic acid, hippuric acid, hydrobromic acid, hydrochloric acid, (+)-L-lactic acid, (+)-DL-lactic acid, lactobionic acid, maleic acid, (−)-L-malic acid, malonic acid, (+)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid,
p-toluenesulfonic acid, trifluoromethylsulfonic acid, and undecylenic acid. Representative bases which may be used in the preparation of pharmaceutically acceptable addition salts include, but are not limited to, the following: ammonia, L-arginine, benethamine, benzathine, calcium hydroxide, choline, dimethylethanolamine, diethanolamine, diethylamine, 2-(diethylamino)-ethanol, ethanolamine, ethylene-diamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide. A particular salt is the trifluoroacetic acid addition salt.

The names of compounds were generated according to the nomenclature rules agreed upon by the Chemical Abstracts Service (CAS) or according to the nomenclature rules agreed upon by the International Union of Pure and Applied Chemistry (IUPAC). In case of tautomeric forms, the name of the depicted tautomeric form of the structure was generated. The other non-depicted tautomeric form is also included within the scope of the present invention.

### Preparation of the Compounds

### Experimental Procedure 1

Final compounds according to Formula (I) and (II) can be prepared by deprotecting intermediate compounds of Formula (III) and (IV) wherein Q represents a base labile (e.g. an acyl) or acid labile (e.g. a trityl) protecting group (Reaction Scheme 1). Such reactions can be performed under art-known reaction conditions.

Alternatively, final compounds according to Formula (I) can be obtained by functional group interconversion of R¹ by art-known methods, such as, for example, exchanging a bromine for a heterocycle by using standard cross coupling reactions, such as, for example, the Suzuki reaction.

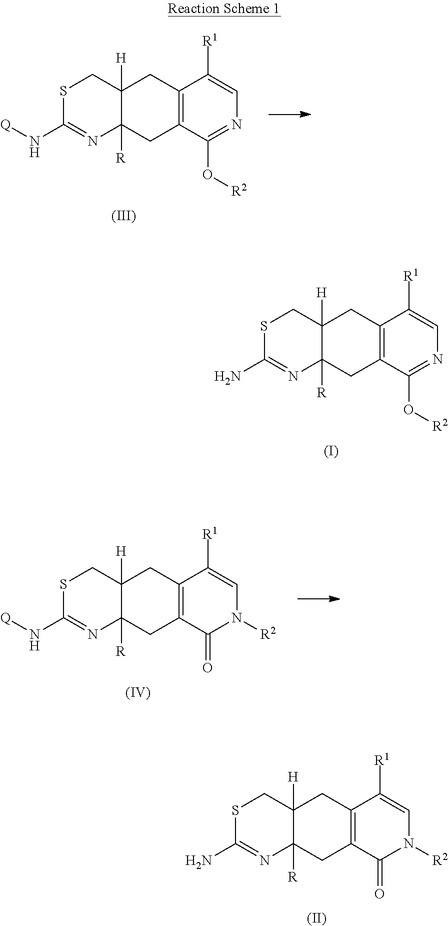

### Preparation of the Intermediate Compounds

### Experimental Procedure 2

Intermediates of Formula (III) or (IV) wherein R^{1a} represents C₁₋₃alkyl, (C₃₋₆cycloalkyl)C₁₋₃alkyl, homoaryl, heteroaryl or heterocyclyl, herein referred to as intermediates of Formula (III-a) and (IV-a) respectively, can be prepared by a Suzuki-Miyaura cross coupling reaction of the corresponding intermediate of Formula (III-b) or (IV-b) wherein R^{1b} represents halo, preferably bromo, with an intermediate of Formula (V) wherein R^{1a} is as defined hereinbefore and R^{a} and R^{b} may be hydrogen or C₁₋₄alkyl, or may be taken together to form a bivalent radical of formula CH₂CH₂, CH₂CH₂CH₂, or C(CH₃)_{2C}(CH₃)₂ (Reaction Scheme 2). The reaction can be performed in a suitable reaction inert solvent, such as, toluene, or mixtures of inert solvents such as, for example, 1,4-dioxane/water in the presence of a suitable base, such as, for example, potassium phosphate tribasic or potassium carbonate, a suitable Pd-complex catalyst such as, for example, palladium (II) acetate, and a suitable ligand, such as, for example, tricyclohexylphosphine, at an elevated temperature in the range of 60 to 120° C. for a period of time to ensure the completion of the reaction. Intermediates of Formula (V) can be obtained commercially or synthesized according to literature procedures.

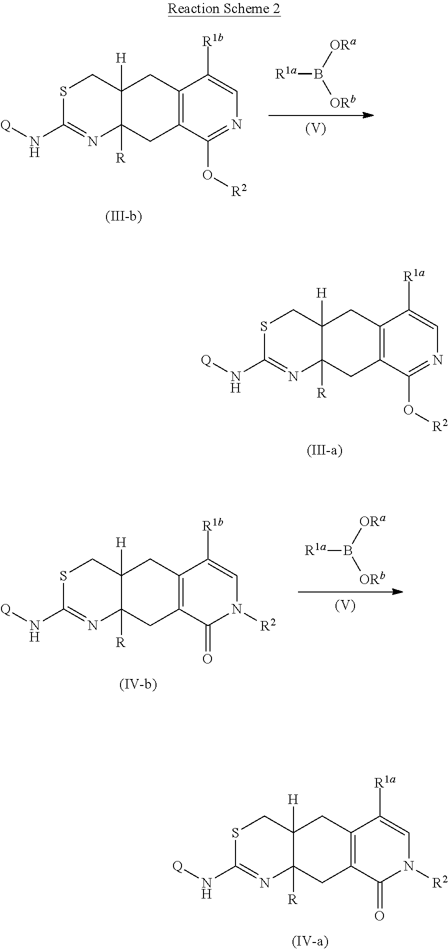

### Experimental Procedure 3

Intermediates of Formula (IV) wherein R² is C₁₋₃alkyl herein referred to as intermediates of Formula (IV-c) can be prepared by reaction the corresponding intermediates of Formula (III) wherein R² is methyl, herein referred to as intermediates of Formula (III-c), with C₁₋₃alkyl iodide (Reaction Scheme 3). The reaction can be performed under thermal conditions such as, for example, heating the reaction mixture at 100° C. In Reaction Scheme 3, all variables are defined as in Formula (I).

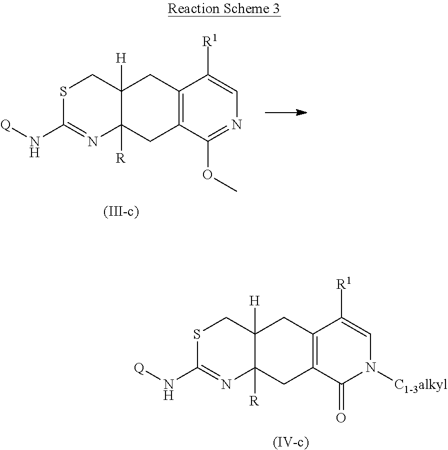

### Experimental Procedure 4

Intermediate compounds of Formula (IV) wherein R² is hydrogen herein referred to as (IV-d) can be prepared from an intermediate compound of Formula (III-c), following art-known O-demethylation procedures. Said transformation may conveniently be conducted by treatment of intermediate (III-c) with a suitable O-demethylating agent, such as, trimethylchlorosilane, in the presence of a suitable additive such as, sodium iodide, in a suitable inert solvent such as, acetonitrile, under suitable reaction conditions, such as at a convenient temperature, typically 50° C., for a period of time to ensure the completion of the reaction. In Reaction Scheme 4, all variables are defined as in Formula (I).

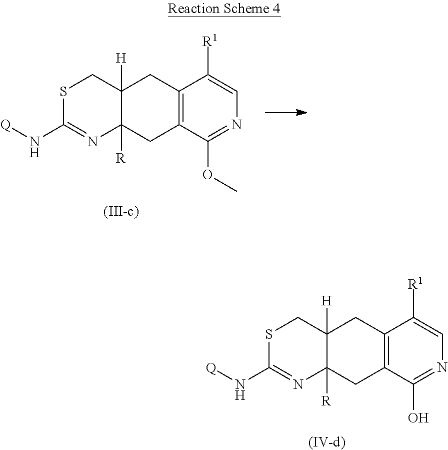

### Experimental Procedure 5

Intermediate compounds of Formula (III) wherein R¹ is cyano herein referred to as (III-d) can be prepared from the corresponding intermediates of Formula (III-b) by art-known cyanation procedures (Reaction Scheme 9). Said cyanation may conveniently be conducted by treatment of the corresponding intermediate compounds of Formula (III-b) with a cyanating agent such as, for example, zinc cyanide in the presence of a suitable Pd catalyst, such as, for example, bis(dibenzylideneacetone)palladium (0), a suitable ligand, such as, for example, 1,1′-bis(diphenylphosphino)ferrocene, and zinc dust in a suitable inert solvent such as, for example, DMA and the like at a suitable temperature such as, for example, 120° C. until completion of the reaction. In Reaction Scheme 5, all variables are defined as in Formula (I).

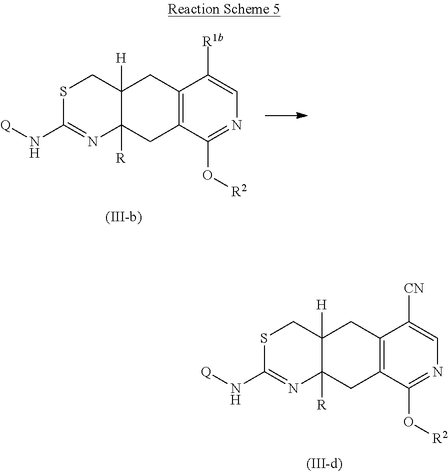

### Experimental Procedure 6

Intermediate compounds of Formula (III) wherein R¹ is C₁₋₃ alkyloxycarbonyl or hydroxycarbonyl, herein referred to as (III-e), can be prepared from the corresponding intermediate compounds of Formula (III-b) following art-known palladium-catalyzed carbonylation procedures (Reaction Scheme 6). Said carbonylation may conveniently be conducted by stirring an intermediate compound of Formula (III-b) under a carbon monoxide atmosphere in the presence of a suitable palladium catalyst, such as, for example, palladium acetate, a suitable ligand, such as, 1,3-bis(diphenylphosphino)propane and a suitable base, such as, potassium acetate in a suitable reaction solvent or mixtures of solvents such as, for example, THF/EtOH. Reaction may be carried out in an autoclave at a suitable pressure such as, for example, 30 bar, at a convenient temperature, typically 120° C., for a period of time to ensure the completion of the reaction. In Reaction Scheme 6, all variables are defined as in Formula (I).

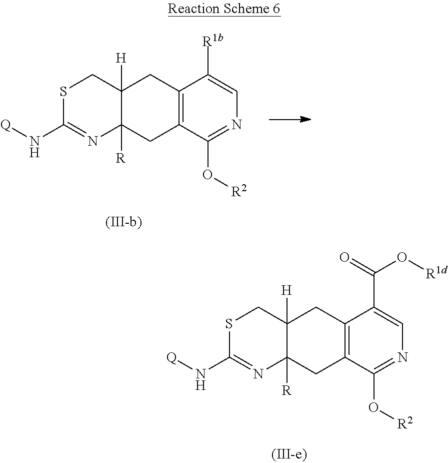

### Experimental Procedure 7

Intermediate compounds of Formula (III-b) can be prepared from an intermediate compound of Formula (III-d) wherein R₁ is hydrogen by art-known bromination procedures. Said bromination may conveniently be conducted by treatment of the corresponding intermediate compounds of Formula (III-f) with a brominating agent such as, for example, N-bromosuccinimide in a suitable inert solvent such as, for example, acetonitrile and the like at a suitable temperature such as, for example, room temperature until completion of the reaction, for example 16 hours.

Intermediates compound of Formula (III-f) may need to be protected by a protecting group PG such as, for example, tert-butoxycarbonyl group, following art-known procedures. Said reaction can conveniently be conducted by treatment of intermediate compound (III-f) with di-tert-butyl dicarbonate, in the presence of a suitable catalyst, such as, 4-(dimethylamino)pyridine (DMAP), in a suitable inert solvent such as, THF, under suitable reaction conditions, such as at a convenient temperature, typically r.t., for a period of time to ensure the completion of the reaction.

The protected intermediate (III-g) may then be brominated as described above to yield (III-h) which than may be deprotected by treatment with a suitable acid, such as for example, trifluoroacetic acid of formic acid in a suitable solvent, or neat, at ambient temperature to yield intermediate (III-b).

In Reaction Scheme 7, Q and PG are a protecting group and all other variables are defined as in Formula (T)

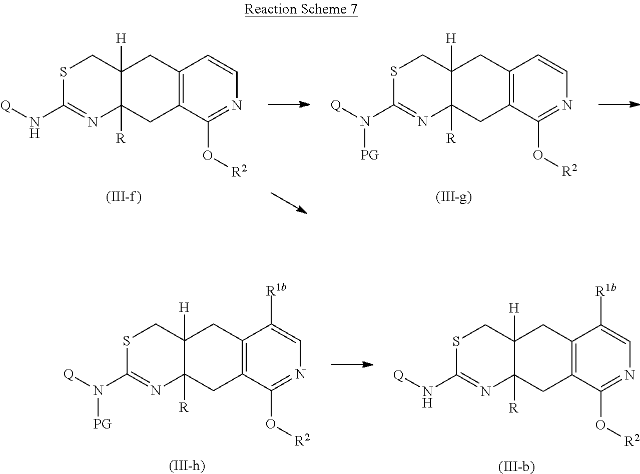

### Experimental Procedure 8

Intermediate compounds of Formula (III) can be prepared from an intermediate compound of Formula (VI) following art-known cyclization procedures. Said cyclization may conveniently be conducted by treatment of an intermediate compound of Formula (VI) with a suitable reagent, such as 1-chloro-N,N-2-trimethylpropenylamine, in a suitable reaction solvent, such as for example DCM under suitable reaction conditions, such as at a convenient temperature, typically r.t., for a period of time to ensure the completion of the reaction.

Intermediate compounds of Formula (VII) can be prepared by reacting the corresponding intermediate compounds of Formula (VIII) with a suitable reagent, such as, benzyl isothiocyanate (resulting in compounds (VI) and (III) wherein Q is phenyl(C═O)—), in a suitable inert solvent, such as, for example, DCM, at a convenient temperature, typically r.t., until completion of the reaction, for example 3 hours.

Intermediate compounds of Formula (VII) can be prepared from the corresponding intermediate compounds of Formula (VIII) following art-known aziridine ring opening procedures. Said reaction may be carried out by stirring the reactants under a hydrogen atmosphere in the presence of an appropriate catalyst such as, for example, Raney-nickel in a suitable solvent, such as, for example, alkanols, e.g. methanol, ethanol and the like, at a convenient temperature, typically r.t., until completion of the reaction, for example 6 hours.

Intermediate compounds of Formula (VIII) can be prepared by reacting the corresponding intermediate compounds of Formula (IX) with an intermediate of Formula (X). The reaction can be performed in a suitable reaction inert solvent, such as, THF under suitable reaction conditions, such as at a suitable temperature, typically in a range between −78° C. and room temperature, for a period of time to ensure the completion of the reaction. An intermediate compound of Formula (X) can be obtained commercially or synthesized according to literature procedures.

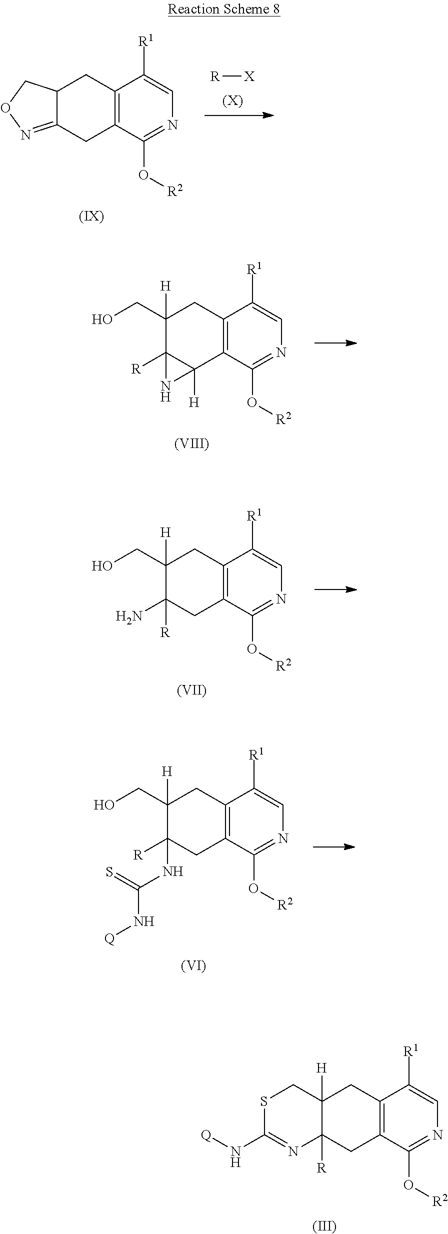

### Experimental Procedure 9

Intermediate compounds of Formula (IX) can be prepared by reacting the corresponding intermediate compounds of Formula (XI) following art-known cyclization procedures. Said cyclization may be conveniently conducted by treatment of an intermediate compound of Formula (XI) with a suitable acid, such as, for example hydrochloric acid, in a suitable reaction inert solvent, such as, THF under suitable reaction conditions, such as at a suitable temperature, typically 50° C., for a period of time to ensure the completion of the reaction.

Intermediate compounds of Formula (XI) can be prepared by reacting the intermediate compounds of Formula (XII) following art-known coupling procedures. Said transformation may be conveniently conducted by conversion of an intermediate compound of Formula (XII) to the corresponding cyanocuprate reagent in the presence of a suitable metalation reagent, such as, isopropylmagnesium chloride lithium chloride complex, and a suitable organocuprate precursor, such as, for example, copper(I) cyanide di(lithium chloride) complex solution, followed by addition of a suitable halide, such as allyl bromide. Reaction may be performed in a suitable inert solvent, such as, for example, THF and the like solvents, at a convenient temperature, typically −70° C.-r.t. for a period of time to ensure the completion of the reaction.

Intermediate compounds of Formula (XII) can be prepared by reacting the intermediate compounds of Formula (XIII) following art-known Wittig reaction procedures. Said reaction may conveniently be conducted by treatment of the intermediate compound of Formula (XIII) with a suitable phosphonium salt, such as, for example, methoxymethyl triphenylphosphonium chloride, in the presence of a suitable base such as, for example, potassium bis(trimethylsilyl)amide, in a suitable reaction-inert solvent, such as, for example, toluene, at convenient temperature, typically −10° C.-r.t., for a period of time to ensure the completion of the reaction. Intermediate compounds of Formula (XIII) can generally be obtained commercially or synthesized according to literature procedures. In Reaction Scheme 9, all variables are defined as in Formula (I)

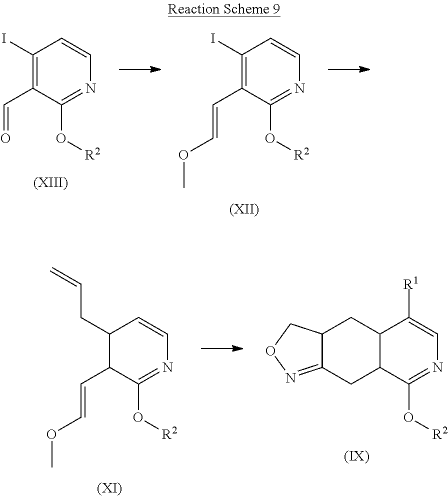

### Experimental Procedure 10

Alternatively, intermediate compounds of Formula (IX) can undergo addition of an organometallic species of Formula (XIV), where R′ is any radical which can be converted into R by using procedures known to the person skilled in the art, such as, for example, cross coupling reactions, alkylation reactions and deprotection reactions. Intermediate compounds (VIII-a) can be carried on in the synthesis using the same synthetic pathway described in the examples before. The person skilled in the art will be able to judge at which point of the synthetic sequence the conversion of R to R is appropriate to perform.

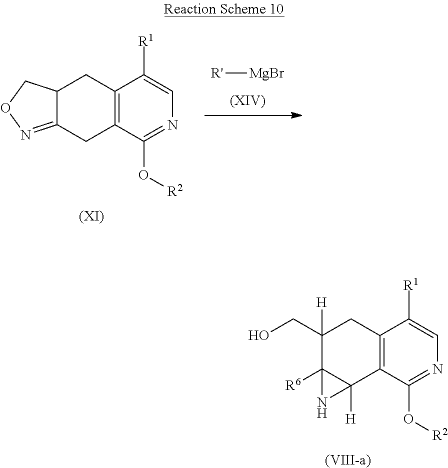

### Preparation of the Compounds—Flow Chemistry

A number of compounds were synthesized and screened using the CyclOps™ platform as described herein, which worked with a high success range (61-96% success rate). The flow synthesis system utilized the Vapourtec® R4 reactors and R2 pump modules with integrated valves and reagent loops controlled by FlowCommander™ software. Up to four reactors, pumps and valves were used depending on the complexity of the chemistry. The output from the final reactor flowed into a HPLC injection valve enabling an aliquot of product to be injected onto the purification system. Loss of material due to dispersion in the synthesis system was minimized in several ways. Firstly small bore tubing was used throughout the system as this minimised dispersion. Secondly, the reagent loop sizes were selected to ensure a steady state concentration of reactants and product was achieved in the reactor. Finally, the injection to HPLC was timed to ensure that an aliquot was taken at the point of maximum product concentration, i.e. under steady state conditions. In general, the use of fresh bottles of reagents and/or generating reagents in situ may improve the synthetic outcome.

### Suzuki Reactions

Compounds of Formula (I), wherein R¹ is heteroaryl, herein referred to as compounds of Formula (I-a), can be prepared by transformations known to the skilled person, such as Suzuki reactions, as shown in Scheme 11. The intermediate of Formula (XIV) was obtained from cleavage of protecting group Q in intermediate of Formula (III-b) analogous to Reaction 1 and it was heated with an appropriate boronic acid or ester of formula (V-a) in a suitable solvent, such as for instance IPA/NMP and THF, using an appropriate catalyst, such as Pd(dppf)Cl₂, in the presence of a suitable base, for example potassium carbonate or DBU, preferably DBU. Water can also be added to assist the outcome of the reaction. The reactions are typically performed under appropriate reaction conditions typically at 150° C. for twenty minutes with a two-fold excess of coupling agent with respect to intermediate (XIV), and three equivalents of base. The reaction mixture is passed through a silica cartridge to remove palladium catalyst before automatic LCMS purification.

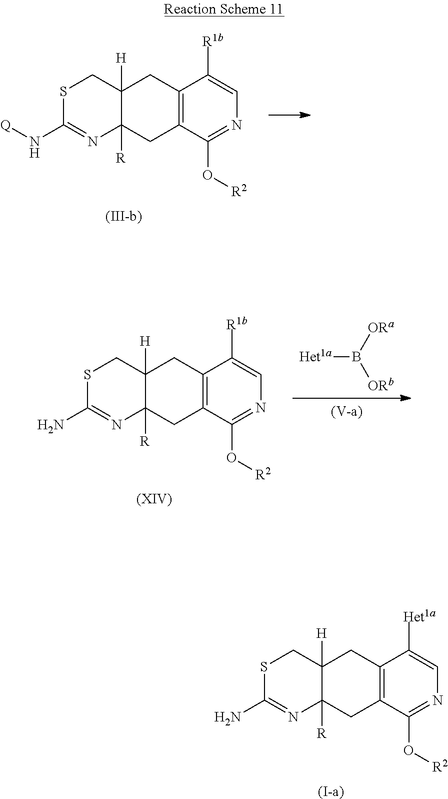

### Alkylation Reaction Followed by Suzuki

Two-step chemistry developed in batch where an amino protected Intermediate (III-i) wherein PG is a base labile protecting group, e.g. Boc, and R is a phenyl having at least a substituent selected from hydroxyl, is first alkylated at the phenol with a suitable alkylating agent in the presence of a base, preferably DBU. Subsequent Suzuki reaction using an appropriate base, such as potassium carbonate affords the final compound as the carbonate can be used to cleave the amino protecting group. This reaction sequence has not yet been attempted in flow but transfer is expected to be routine.

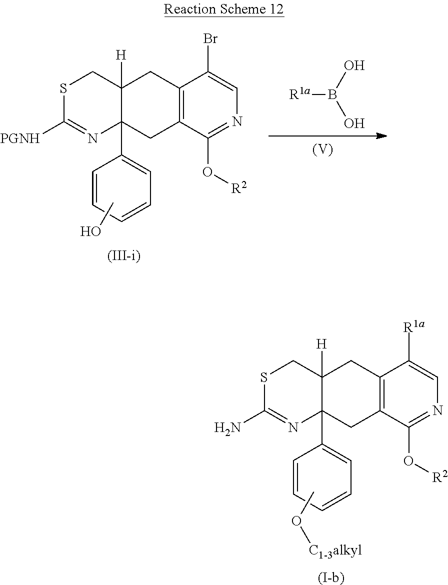

Intermediate of Formula (III-i) is a useful and versatile intermediate in the synthesis of the compounds of the invention. Thus in an embodiment, the invention relates to a compound of Formula (III-i′)

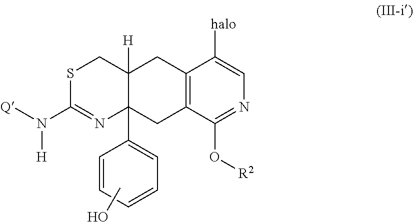

wherein Q′ is H or a protecting group, halo is bromo or chloro, in particular bromo, and R² is as defined for the compounds of Formula (I) herein.

### Pharmacology

The compounds of the present invention and the pharmaceutically acceptable compositions thereof inhibit BACE and therefore may be useful in the treatment or prevention of Alzheimer's Disease (AD), mild cognitive impairment (MCI), senility, dementia, dementia with Lewy bodies, cerebral amyloid angiopathy, multi-infarct dementia, Down's syndrome, dementia associated with Parkinson's disease, dementia of the Alzheimer's type, vascular dementia, dementia due to HIV disease, dementia due to head trauma, dementia due to Huntington's disease, dementia due to Pick's disease, dementia due to Creutzfeldt-Jakob disease, frontotemporal dementia, dementia pugilistica, dementia associated with beta-amyloid and age related macular degeneration, type 2 diabetes and other metabolic disorders.

As used herein, the term “treatment” is intended to refer to all processes, wherein there may be a slowing, interrupting, arresting or stopping of the progression of a disease or an alleviation of symptoms, but does not necessarily indicate a total elimination of all symptoms.

The invention also relates to a compound according to the general Formula (I), a stereoisomeric form thereof or a pharmaceutically acceptable acid or base addition salt thereof, for use in the treatment or prevention of diseases or conditions selected from the group consisting of AD, MCI, senility, dementia, dementia with Lewy bodies, cerebral amyloid angiopathy, multi-infarct dementia, Down's syndrome, dementia associated with Parkinson's disease, dementia of the Alzheimer's type, dementia associated with beta-amyloid and age related macular degeneration, type 2 diabetes and other metabolic disorders.

The invention also relates to a compound according to the general Formula (I), a stereoisomeric form thereof or a pharmaceutically acceptable acid or base addition salt thereof, for use in the treatment, prevention, amelioration, control or reduction of the risk of diseases or conditions selected from the group consisting of AD, MCI, senility, dementia, dementia with Lewy bodies, cerebral amyloid angiopathy, multi-infarct dementia, Down's syndrome, dementia associated with Parkinson's disease, dementia of the Alzheimer's type, dementia associated with beta-amyloid and age related macular degeneration, type 2 diabetes and other metabolic disorders.

As already mentioned hereinabove, the term “treatment” does not necessarily indicate a total elimination of all symptoms, but may also refer to symptomatic treatment in any of the disorders mentioned above. In view of the utility of the compound of Formula (I), there is provided a method of treating subjects such as warm-blooded animals, including humans, suffering from or a method of preventing subjects such as warm-blooded animals, including humans, suffering from any one of the diseases mentioned hereinbefore.

Said methods comprise the administration, i.e. the systemic or topical administration, preferably oral administration, of a therapeutically effective amount of a compound of Formula (I), a stereoisomeric form thereof, a pharmaceutically acceptable addition salt or solvate thereof, to a subject such as a warm-blooded animal, including a human.

Therefore, the invention also relates to a method for the prevention and/or treatment of any of the diseases mentioned hereinbefore comprising administering a therapeutically effective amount of a compound according to the invention to a subject in need thereof.

The invention also relates to a method for modulating beta-site amyloid cleaving enzyme activity, comprising administering to a subject in need thereof, a therapeutically effective amount of a compound according to the invention and as defined in the claims or a pharmaceutical composition according to the invention and as defined in the claims.

A method of treatment may also include administering the active ingredient on a regimen of between one and four intakes per day. In these methods of treatment the compounds according to the invention are preferably formulated prior to administration. As described herein below, suitable pharmaceutical formulations are prepared by known procedures using well known and readily available ingredients.

The compounds of the present invention, that can be suitable to treat or prevent Alzheimer's disease or the symptoms thereof, may be administered alone or in combination with one or more additional therapeutic agents. Combination therapy includes administration of a single pharmaceutical dosage formulation which contains a compound of Formula (I) and one or more additional therapeutic agents, as well as administration of the compound of Formula (I) and each additional therapeutic agent in its own separate pharmaceutical dosage formulation. For example, a compound of Formula (I) and a therapeutic agent may be administered to the patient together in a single oral dosage composition such as a tablet or capsule, or each agent may be administered in separate oral dosage formulations.

A skilled person will be familiar with alternative nomenclatures, nosologies, and classification systems for the diseases or conditions referred to herein. For example, the fifth edition of the Diagnostic & Statistical Manual of Mental Disorders (DSM-5™) of the American Psychiatric Association utilizes terms such as neurocognitive disorders (NCDs) (both major and mild), in particular, neurocognitive disorders due to Alzheimer's disease, due to traumatic brain injury (TBI), due to Lewy body disease, due to Parkinson's disease or to vascular NCD (such as vascular NCD present with multiple infarctions). Such terms may be used as an alternative nomenclature for some of the diseases or conditions referred to herein by the skilled person.

### Pharmaceutical Compositions

The present invention also provides compositions for preventing or treating diseases in which inhibition of beta-secretase is beneficial, such as Alzheimer's disease (AD), mild cognitive impairment, senility, dementia, dementia with Lewy bodies, Down's syndrome, dementia associated with stroke, dementia associated with Parkinson's disease and dementia associated with beta-amyloid and age related macular degeneration, type 2 diabetes and other metabolic disorders. Said compositions comprising a therapeutically effective amount of a compound according to formula (I) and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy. A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The exact dosage and frequency of administration depends on the particular compound of formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

The present compounds can be used for systemic administration such as oral, percutaneous or parenteral administration; or topical administration such as via inhalation, a nose spray, eye drops or via a cream, gel, shampoo or the like. The compounds are preferably orally administered. The exact dosage and frequency of administration depends on the particular compound according to formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The amount of a compound of Formula (I) that can be combined with a carrier material to produce a single dosage form will vary depending upon the disease treated, the mammalian species, and the particular mode of administration. However, as a general guide, suitable unit doses for the compounds of the present invention can, for example, preferably contain between 0.1 mg to about 1000 mg of the active compound. A preferred unit dose is between 1 mg to about 500 mg. A more preferred unit dose is between 1 mg to about 300 mg. Even more preferred unit dose is between 1 mg to about 100 mg. Such unit doses can be administered more than once a day, for example, 2, 3, 4, 5 or 6 times a day, but preferably 1 or 2 times per day, so that the total dosage for a 70 kg adult is in the range of 0.001 to about 15 mg per kg weight of subject per administration. A preferred dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

For the compositions, methods and kits provided above, one of skill in the art will understand that preferred compounds for use in each are those compounds that are noted as preferred above. Still further preferred compounds for the compositions, methods and kits are those compounds provided in the non-limiting Examples below.

### Experimental Part

Hereinafter, the term “aq.” means aqueous, “r.m.” means reaction mixture, “r.t.” means room temperature, “DIPEA” means N,N-diisopropylethylamine, “DIPE” means diisopropylether, “THF” means tetrahydrofuran, “DMF” means dimethylformamide, “DCM” means dichloromethane, “EtOH” means ethanol “EtOAc” means ethylacetate, “AcOH” means acetic acid, “iPrOH” means isopropanol, “iPrNH₂” means isopropylamine, “MeCN” means acetonitrile, “MeOH” means methanol, “Pd(OAc)₂” means palladium(II)diacetate, “rac” means racemic, “sat.” means saturated, “SFC” means supercritical fluid chromatography, “SFC-MS” means supercritical fluid chromatography/mass spectrometry, “LC-MS” means liquid chromatography/mass spectrometry, “GCMS” means gas chromatography/mass spectrometry, “HPLC” means high-performance liquid chromatography, “RP” means reversed phase, “UPLC” means ultra-performance liquid chromatography, “Rt” means retention time (in minutes), “[M+H]⁺” means the protonated mass of the free base of the compound, “DAST” means diethylaminosulfur trifluoride, “DMTMM” means 4-(4,6-dimethoxy-1,3,5-triazin-2-yl)-4-methylmorpholinium chloride, “HATU” means O-(7-azabenzotriazol-1-yl)-N,N,N′,N′-tetramethyluronium hexafluorophosphate, “HBTU” means N,N,N′,N′-Tetramethyl-O-(1H-benzotriazol-1-yl)uronium hexafluorophosphate, “Xantphos” means (9,9-dimethyl-9H-xanthene-4,5-diyl)bis[diphenylphosphine], “TFA” means trifluoroacetic acid, “Et₂O” means diethylether, “DMSO” means dimethylsulfoxide, “NMR” means nuclear magnetic resonance, “LDA” means lithium diisopropylamide, “DIPA” means diisopropylamine, “n-BuLi” means n-butyllithium. “h” means hours. “min” means minutes, “Na₂CO₃” means sodium carbonate, “NaHCO₃” means sodium bicarbonate, “sol.” means solution, “MgSO₄” means magnesium sulfate, “NH₄Cl” means ammonium chloride, “BOC” means t-butoxycarbonyl, “DMAP” means dimethylaminopyridine, “NBS” means N-bromosuccinimide, “Pd(PPh₃)₄” means tetrakis(triphenylphosphine)palladium(0), “DBU” means 1,8-diazabicyclo[5.4.0]undec-7-ene, “SQD” means Single Quadrupole Detector, “MSD” means Mass Selective Detector, “BEH” means bridged ethylsiloxane/silica hybrid, “DAD” means Diode Array Detector, “HSS” means High Strength silica., “Q-Tof” means Quadrupole Time-of-flight mass spectrometers, “CLND” means ChemiLuminescent Nitrogen Detector, and “ELSD” means Evaporative Light Scanning Detector.

### Assignment and Graphical Representation of Stereochemical Configuration

The stereoconfiguration of centres C₄ₐ and C₁₀ₐ of intermediates/compounds has been represented as follows:

a) when the intermediate/compound is enantiopure and the absolute stereoconfiguration is known, the core has been represented as

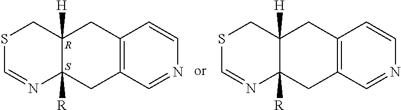

when for instance, the stereoconfiguration corresponds with C₄ₐ (R), C₁₀ₐ(S) and the compound is a single diastereoisomer and enantiopure;
b) when the intermediate/compound is enantiopure but the absolute stereoconfiguration has not been determined, the core has been represented as

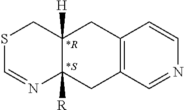

(wherein the wedges have been assigned at random to indicate the cis diastereoisomer); when the other pure enantiomer of cis relative configuration has been isolated, the intermediate/compound has been represented as

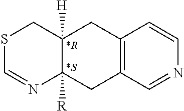

in order to differentiate from the other isolate enantiopure intermediate/compound;
c) when the intermediate/compound is a racemic mixture of two enantiomers of cis relative configuration, the core has been represented as

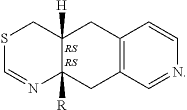

The absolute stereochemical configuration of intermediates/compounds has been rationalized on the basis of chemical synthetic methods and NMR (assignment of relative stereoconfiguration) and co-crystallisation of compounds 2, 3, 16, 25, 87-89 and 200, as well as other enantiopure analogues, with BACE 1 enzymes, which enabled ascertaining the preferred orientation of the R group in the compounds, together with the exhibited in vitro activity of the compounds.

### A. Preparation of the Intermediates

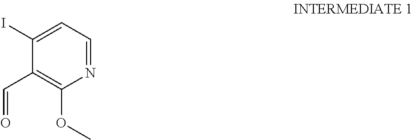

A mixture of DIPA (3.5 mL, 25 mmol) in THF (100 mL) was cooled to −20° C. and n-BuLi (2.7 M in heptane, 9.2 mL, 25 mmol) was added dropwise. After stirring 10 min, the r.m. was cooled to −75° C. and 2-fluoro-3-iodopyridine (5.55 g, 25 mmol) in THF (50 mL) was added dropwise. Stirring was continued for 2 h at −65° C. The r.m. was cooled to −75° C. and ethyl formate (2.3 mL, 28 mmol) in THF (25 mL) was added dropwise. After 10 min sodium methoxide (5.8 mL, 0.95 g/mL, 25 mmol, 25% purity) was added dropwise. The cooling bath was removed and the r.m. was allowed to come to r.t. and treated with brine (50 mL), Et₂O (100 mL) and the layers were separated. The aq. layer was extracted with Et₂O (100 mL) and the combined organic layers were treated with brine (50 mL), dried over MgSO₄, filtered and concentrated in vacuo to afford intermediate 1 (6.15 g, 94%), which was used as such in the next reaction step.

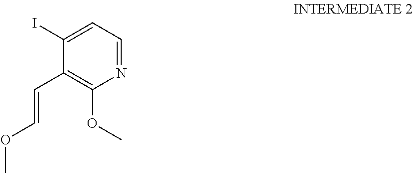

To a stirred mixture of methoxymethyl triphenylphosphonium chloride (8.4 g, 24 mmol) in toluene (150 mL) was added potassium bis(trimethylsilyl)amide (0.7 M in toluene, 34 mL, 24 mmol) dropwise at −10° C. Stirring was continued for 30 min at this temperature. Intermediate 1 (2.1 g, 8 mmol) in toluene (20 mL) was added dropwise and after 2 h the r.m. was quenched with water (50 mL) and the layers were separated. The organic layer was dried over MgSO₄, filtered and concentrated in vacuo to afford a tan oil. This oil was purified by flash chromatography (silica, EtOAc/heptane 0/100 to 10/90) to afford intermediate 2 as an oil (1.86 g, 80%).

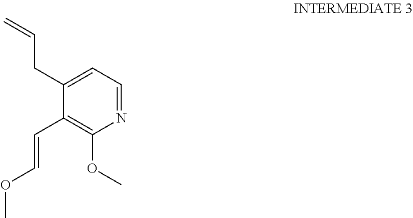

To a stirred and cooled (−70° C.) mixture of intermediate 2 (30 g, 100 mmol) in THF (500 mL) was added dropwise isopropylmagnesium chloride-lithium chloride complex (105 mL, 1.3 M, 140 mmol) while keeping the internal temperature below −65° C. When addition was complete, stirring was continued for 1.5 h. Copper(I) cyanide di(lithium chloride) complex sol. (105 mL, 1 M, 110 mmol) was then added dropwise at −70° C. and after 15 min allyl bromide (28 mL, 31 mmol) was added dropwise. The r.m. was allowed to come to r.t. and then quenched with brine (100 mL), diluted with Et₂O (0.3 L) and water (0.1 L) and the layers were separated. The organic layer was washed first portionwise with ammonia until the blue colour disappeared (5×0.2 L) and then with brine (0.1 L). The organic layer was dried over MgSO₄, filtered and concentrated in vacuo to afford a residue which was purified by column chromatography (silica, DCM/heptane 98/2 to 100/0) to afford intermediate 3 (19.6 g, 93%).

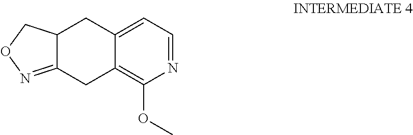

A stirred sol. of intermediate 3 (19.6 g, 95 mmol) in THF (200 mL) was treated with aq. 6 M HCl (70 mL, 420 mmol) and the r.m. was heated at 50° C. for 30 min. The r.m was poured into ice water (0.2 L) and treated with sat. Na₂CO₃ sol. until neutral pH. The r.m. was extracted with DCM (3×0.1 L) and the combined organic layers were dried over MgSO₄. To the resulting sol. was added triethylamine (40 mL, 290 mmol) and then hydroxylamine hydrochloride (8 g, 120 mmol) and stirring was continued for 1 h. The r.m. was diluted with sat. NaHCO₃ sol. (0.1 L) and the layers were separated. The organic layer was dried over MgSO₄, filtered and transferred to a 1 L 4 neck flask, equipped with a mechanical stirrer and cooled to 0° C. (internal temperature). To this cooled sol., sodium hypochlorite (210 mL, 470 mmol) was added dropwise. After complete addition, the r.m. was allowed to come to r.t. and stirring was continued at r.t. overnight. The layers were separated and the aq. layer was extracted with DCM (0.2 L). The combined organic layers were dried over MgSO₄, filtered and concentrated in vacuo to give a solid which was recrystallized from DIPE (0.1 L) to afford intermediate 4 (8.64 g, 44%).

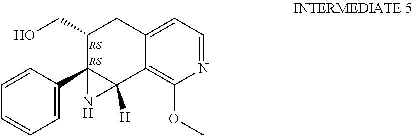 [SMILES: COc1nccc2c1[C@@H]1N[C@]1(c1ccccc1)[C@H](CO)C2]

To a stirred, cooled (−70° C.) sol. of intermediate 4 (0.35 g, 1.71 mmol) in THF (20 mL) was added dropwise phenylmagnesium bromide in THF (8.6 mL, 1 M, 8.6 mmol) and the r.m. was kept at this temperature for 1 h, then it was allowed to come to r.t. and stirring was continued overnight. The r.m. was quenched with aq. sat. NH₄Cl sol. (5 mL), EtOAc (10 mL) and the layers were separated. The aq. layer was extracted with EtOAc (2×5 mL) and the combined organic layers were treated with brine (10 mL), dried over MgSO₄, filtered and concentrated in vacuo. The crude was purified by flash chromatography (silica, 7 M ammonia in MeOH/DCM 0/100 to 5/100) to give a residue (0.35 g), which was further purified by flash chromatography (silica, EtOAc/heptane 20/80 to 90/100) to give intermediate 5 as a white solid (0.14 g, 28%, cis/trans 85/15).

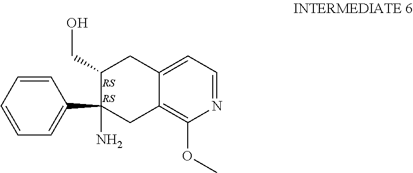 [SMILES: COc1nccc2c1C[C@@](N)(c1ccccc1)[C@H](CO)C2]

A hydrogenation flask was charged with Raney nickel (0.15 g, 2.6 mmol), EtOH (35 mL) and intermediate 5 (0.15 g, 0.51 mmol). The r.m. was stirred under hydrogen atmosphere for 6 h, then filtered over a small plug of diatomaceous earth and concentrated in vacuo. The crude was purified by flash chromatography (silica, 7 M ammonia in MeOH/DCM 0/100 to 5/95) to afford intermediate 6 (0.15 g, 99%, cis/trans 96/4).

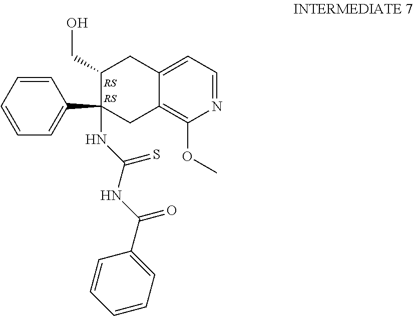 [SMILES: COc1nccc2c1C[C@@](NC(=S)NC(=O)c1ccccc1)(c1ccccc1)[C@H](CO)C2]

A stirred sol. of intermediate 6 (0.15 g, 0.53 mmol) in DCM (5.2 mL) was treated with benzoyl isothiocyanate (120 mg, 0.74 mmol). After 3 h at r.t. the r.m. was treated with water (1 mL) and the layers were separated. The organic layer was dried over MgSO₄, filtered and concentrated in vacuo. The crude was purified by flash chromatography (silica, MeOH/DCM 0/100 to 5/95) to afford intermediate 7 (0.13 g, 53%, cis).

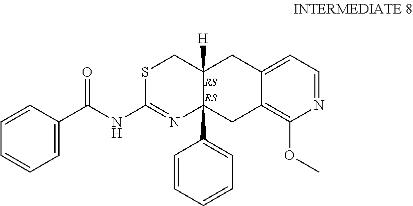 [SMILES: COc1nccc2c1C[C@]1(c3ccccc3)N=C(NC(=O)c3ccccc3)SC[C@@H]1C2]

A stirred sol. of intermediate 7 (0.13 g, 0.29 mmol) in DCM (20 mL) was treated with 1-chloro-N,N-2-trimethylpropenylamine (58 μL, 0.44 mmol) and the ensuing r.m. was stirred at r.t. overnight. Sat. aq. NaHCO₃ sol. (6 mL) was added and the layers were separated. The organic layer was dried over MgSO₄, filtered and concentrated in vacuo to give an oil. The crude was purified by flash chromatography (silica, EtOAc/heptane 0/100 to 40/60) to afford intermediate 8 as a white solid (0.1 g, 83%, cis).

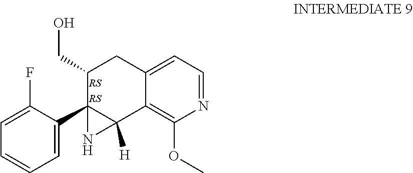 [SMILES: COc1nccc2c1[C@@H]1N[C@]1(c1ccccc1F)[C@H](CO)C2]

To a stirred, cooled (5° C.) heterogeneous mixture of 2-fluorophenylmagnesium bromide (20 mL, 1 M, 20 mmol) was added dropwise a sol. of intermediate 4 (2 g, 9.8 mmol) in toluene (40 mL). When addition was complete, stirring was continued for 30 min and then the r.m. was quenched with sat. aq. NH₄Cl sol. (50 mL), water (0.1 L) and the layers were separated. The aq. phase was extracted with EtOAc (3×0.1 L) and the combined organic layers were treated with brine (0.1 L), dried over MgSO₄, filtered and concentrated in vacuo to give a residue which was purified by column chromatography (silica, EtOAc/DCM 0/100 to 100/0) to afford intermediate 9 as an off white foam (2.45 g, 83%, cis/trans 93/7).

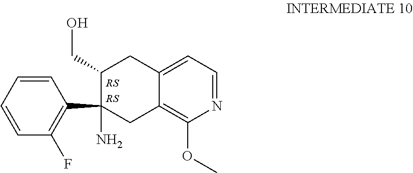 [SMILES: COc1nccc2c1C[C@@](N)(c1ccccc1F)[C@H](CO)C2]

Intermediate 10 was prepared following a synthetic procedure similar to the one reported for the synthesis of intermediate 6. Starting from intermediate 9 (2.8 g, 9.32 mmol) intermediate 10 was obtained and used as such in the next step (2.8 g, quantitative, cis/trans 96/4).

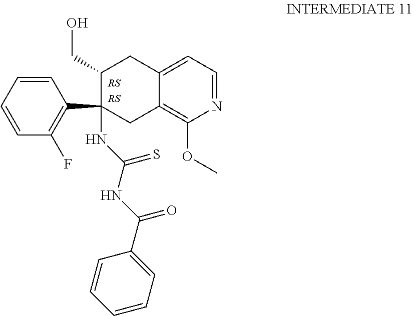 [SMILES: COc1nccc2c1C[C@@](NC(=S)NC(=O)c1ccccc1)(c1ccccc1F)[C@H](CO)C2]

Intermediate 11 was prepared following a synthetic procedure similar to the one reported for the synthesis of intermediate 7. Starting from intermediate 10 (1.6 g, 5.29 mmol) intermediate 11 was obtained as a white foam (2.22 g, 90%, cis).

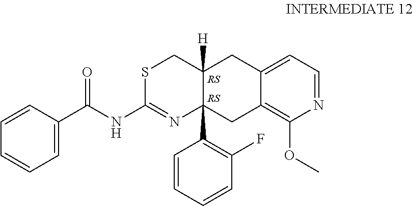 [SMILES: COc1nccc2c1C[C@]1(c3ccccc3F)N=C(NC(=O)c3ccccc3)SC[C@@H]1C2]

Intermediate 12 was prepared following a synthetic procedure similar to the one reported for the synthesis of intermediate 8. Starting from intermediate 11 (2.22 g, 4.77 mmol) intermediate 12 was obtained as a white solid (1.4 g, 66%, cis).

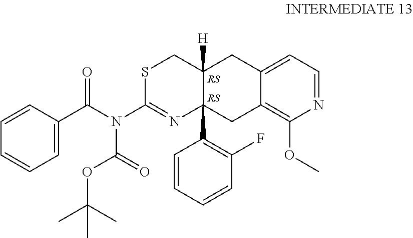 [SMILES: COc1nccc2c1C[C@]1(c3ccccc3F)N=C(N(C(=O)OC(C)(C)C)C(=O)c3ccccc3)SC[C@@H]1C2]

To a stirred mixture of intermediate 12 (1.5 g, 3.4 mmol) in THF (20 mL) was added BOC-anhydride (0.9 g, 4.1 mmol) and DMAP (0.01 g, 0.082 mmol) and the r.m. was stirred at r.t. for 3 h. The r.m. was diluted with sat. aq. NaHCO₃ sol. (5 mL), the layers were separated and the organic layer was dried over MgSO₄, filtered and concentrated in vacuo. The residue was purified by column chromatography (silica, MeOH/DCM 0/100 to 2/98) to afford intermediate 13 as a white solid (1.1 g, 60%, cis).

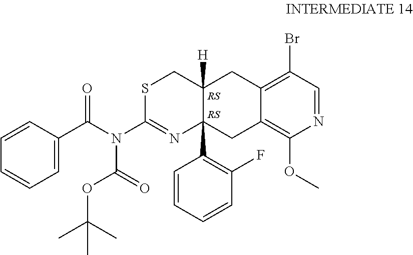 [SMILES: COc1ncc(Br)c2c1C[C@]1(c3ccccc3F)N=C(N(C(=O)OC(C)(C)C)C(=O)c3ccccc3)SC[C@@H]1C2]

To a stirred suspension of intermediate 13 (1.1 g, 2 mmol) in MeCN (50 mL) was added NBS (0.46 g, 2.6 mmol) in small portions. After 16 h, the r.m. was diluted with DCM (0.1 L) and sat. aq. NaHCO₃ sol. and the layers were separated. The organic layer was treated with brine, dried over MgSO₄, filtered and concentrated in vacuo to give a solid. This crude was purified by column chromatography (silica, EtOAc/heptane 0/100 to 50/50) to afford intermediate 14 as a white solid (0.8 g, 64%, cis).

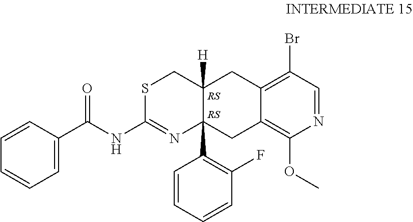 [SMILES: COc1ncc(Br)c2c1C[C@]1(c3ccccc3F)N=C(NC(=O)c3ccccc3)SC[C@@H]1C2]

A stirred mixture of intermediate 14 (0.8 g, 1.3 mmol) in DCM (20 mL) was treated with TFA (1 mL, 13 mmol). After 1 h at r.t. the mixture was diluted with sat. aq. NaHCO₃ sol. until pH ˜8 and the layers were separated. The organic layer was dried over MgSO₄, filtered and concentrated in vacuo to give intermediate 15 as a white solid (0.67 g, 99%, cis).

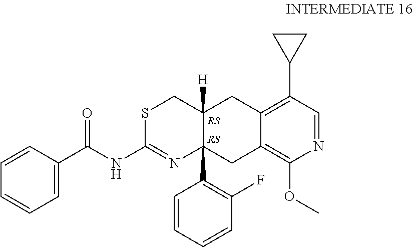 [SMILES: COc1ncc(C2CC2)c2c1C[C@]1(c3ccccc3F)N=C(NC(=O)c3ccccc3)SC[C@@H]1C2]

A microwave tube was charged with intermediate 15 (150 mg, 0.29 mmol), cyclopropylboronic acid (35 mg, 0.41 mmol), tricyclohexylphosphine (10 mg, 0.036 mmol), potassium phosphate tribasic (200 mg, 0.94 mmol), palladium (II) acetate (5 mg, 0.022 mmol), toluene (5 mL) and water (0.1 mL). The r.m. was purged with nitrogen under vigorously stirring for 5 min, then the tube was capped and heated for 3 h at 120° C. in a DrySyn metal heating block. The r.m. was allowed to cool down, diluted with water (5 mL) and toluene (20 mL). The layers were separated and the organic layer was subsequently treated with brine (10 mL), dried over MgSO₄, filtered and concentrated in vacuo. The crude was purified by flash chromatography (silica, EtOAc/heptane 0/100 to 40/60) to afford intermediate 16 as a solid (72 mg, 52%, cis).

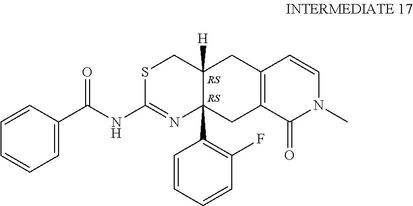 [SMILES: Cn1ccc2c(c1=O)C[C@]1(c3ccccc3F)N=C(NC(=O)c3ccccc3)SC[C@@H]1C2]

A microwave tube was charged with intermediate 12 (0.2 g, 0.45 mmol) and methyl iodide (2 mL, 32 mmol). The tube was capped and heated at 100° C. in a DrySyn metal heating block for 16 h, then the r.m. was concentrated in vacuo. The crude was diluted with DCM (10 mL) and water (1 mL). The layers were separated and the organic layer was dried over MgSO₄, filtered and concentrated in vacuo affording a tan solid. This solid was purified by preparative HPLC (Stationary phase: RP XBridge Prep C18 OBD-10 μm, 30×150 mm, Mobile phase: 0.25% NH₄HCO₃ sol. in water, MeOH) to afford intermediate 17 as a white solid (0.08 g, 40%, cis).

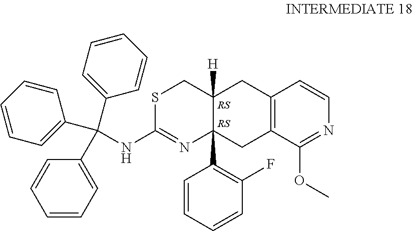 [SMILES: COc1nccc2c1C[C@]1(c3ccccc3F)N=C(NC(c3ccccc3)(c3ccccc3)c3ccccc3)SC[C@@H]1C2]

To a stirred sol. of compound 2 (104 mg, 0.3 mmol) and triphenylmethyl chloride (115 mg, 0.4 mmol) in DMF (2 mL) was added triethylamine (65 μL, 0.47 mmol). The mixture was heated at 80° C. for 4 h. The cooled mixture was poured onto ice water (˜10 mL) and then filtered. The remaining solid was dissolved in DCM (20 mL), dried over MgSO₄, filtered and concentrated in vacuo to give a tan oil. This crude was purified by column chromatography (silica, MeOH/DCM 0/100 to 5/95) to afford intermediate 18 as a white solid (0.14 g, 80%, cis).

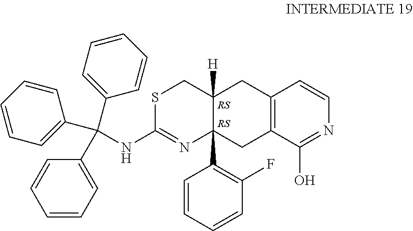 [SMILES: Oc1nccc2c1C[C@]1(c3ccccc3F)N=C(NC(c3ccccc3)(c3ccccc3)c3ccccc3)SC[C@@H]1C2]

A microwave tube charged with a mixture of intermediate 18 (0.14 g, 0.24 mmol) in MeCN (15 mL) was treated with trimethylchlorosilane (0.12 mL, 0.97 mmol) and sodium iodide (0.15 g, 0.98 mmol). The tube was capped and heated at 50° C. for 3 days. The r.m. was diluted with DCM (50 mL) and water (10 mL) and the layers were separated. The organic layer was treated with brine (10 mL), dried over MgSO₄, filtered and concentrated in vacuo to give a dark brown oil. This crude was purified by column chromatography (silica, MeOH/DCM 0/100 to 10/90) to give intermediate 19 as a yellowish solid (0.11 g, 81%, cis).

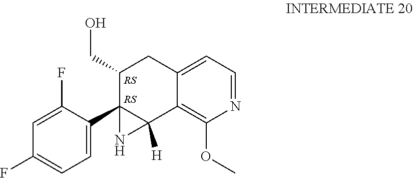 [SMILES: COc1nccc2c1[C@@H]1N[C@]1(c1ccc(F)cc1F)[C@H](CO)C2]

1-Bromo-2,4-difluorobenzene (9.699 mL, 70.51 mmol) was stirred in 43 mL of THF under nitrogen and the r.m. was cooled to −15° C. Isopropylmagnesium chloride (2 M in THF, 43.048 mL, 86.1 mmol) was added dropwise at −15° C. and the r.m. was stirred at 0-5° C. for 1 h, then cooled again to −15° C. Intermediate 4 (7.2 g, 35.26 mmol) dissolved in 43 ml of THF was added dropwise. The mixture was allowed to reach r.t. then added dropwise to 60 mL of NH₄Cl sat. sol. and extracted with EtOAc. The organic layer was dried over MgSO₄, filtered and concentrated in vacuo to give intermediate 20 (11.15 g, 99%, cis/trans mixture).

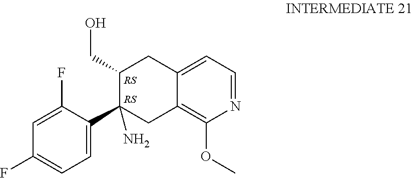 [SMILES: COc1nccc2c1C[C@@](N)(c1ccc(F)cc1F)[C@H](CO)C2]

Raney®-Nickel (64 g) and thiophene (4% in DIPE, 85 mL) in EtOH (473 mL) were placed in a hydrogenation flask before intermediate 20 (17.2 g, 54 mmol) dissolved in EtOH (473 mL) was added. The flask was degassed and then flushed with hydrogen gas before being stirred for 6 h at 14° C. The r.m. was filtered over Dicalite® and washed with EtOH and THF before the product was concentrated by evaporation. The product was purified (silica, MeOH/DCM 0/100 to 6/94). The pure fractions were evaporated to yield intermediate 21 (10.34 g, 60%).

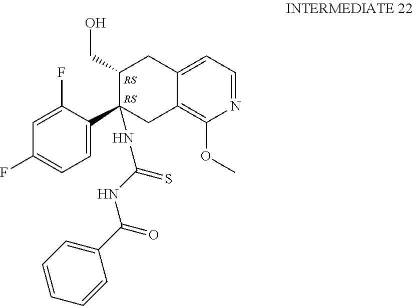 [SMILES: COc1nccc2c1C[C@@](NC(=S)NC(=O)c1ccccc1)(c1ccc(F)cc1F)[C@H](CO)C2]

Intermediate 21 (2.32 g, 7.24 mmol) was dissolved in 130 mL of DCM in an ice bath before benzoyl isothiocyanate (1.66 g, 10.14 mmol) in 20 mL of DCM was added dropwise to the mixture and the reaction was allowed to stir at r.t. for 1.5 h. A small amount of ice was added to the still stirring r.m. and the product was extracted using DCM; the organic layer was dried over MgSO₄, filtered and concentrated by evaporation. The organic layer was purified by column chromatography (silica, EtOAc/heptane 0/100 to 80/20). The fractions containing product were collected and concentrated by evaporation to yield intermediate 22 (3.50 g, quantitative).

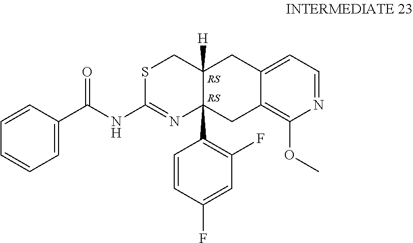 [SMILES: COc1nccc2c1C[C@]1(c3ccc(F)cc3F)N=C(NC(=O)c3ccccc3)SC[C@@H]1C2]

Intermediate 22 (3.34 g, 6.91 mmol) was stirred in DCM (100 mL) at r.t. under a flow of nitrogen before 1-chloro-N,N,2-trimethylpropenylamine (2.5 mL, 18.90 mmol) was added dropwise and the reaction mixture was allowed to stir for 10 min. The reaction went to completion and was then quenched with 20 mL of sat. aq. Sol. NaHCO₃ and allowed to stir for 10 min. The organic material was extracted using DCM, dried over MgSO₄, filtered and concentrated by evaporation. This material was stirred in DIPE in an ice-EtOH bath, to afford a white solid which was filtered off and dried in the oven to yield intermediate 23 (2.5 g, 78%).

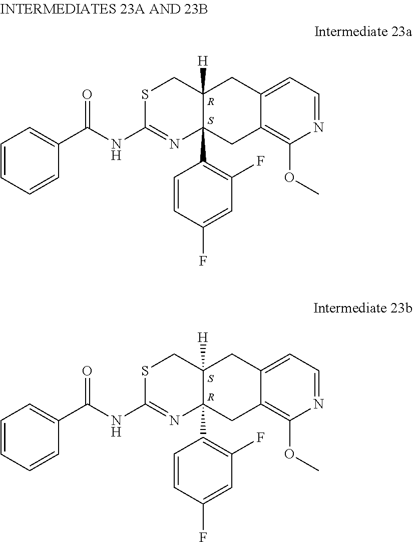

Intermediate 22 (3.5 g, 7.24 mmol) was stirred in DCM (91 mL) at r.t. under a flow of nitrogen before 1-chloro-N,N,2-trimethylpropenylamine (2.62 mL, 19.80 mmol) was added dropwise and the r.m. was allowed to stir for 10 min. The reaction went to completion and was then quenched with 20 mL of sat. aq. sol. NaHCO₃ and allowed to stir for 10 min. The organic material was extracted using DCM, dried over MgSO₄, filtered and concentrated by evaporation. This material was stirred in DIPE to afford a white solid which was filtered off and dried in the oven to yield 2.46 g of a mixture which was purified by Prep SFC (Stationary phase: Chiralpak Diacel AD 30×250 mm, mobile phase: CO₂, MeOH with 0.2% iPrNH₂) to yield intermediate 23a (1.99 g, 33%, pure enantiomer) and intermediate 23b (1.67, 28% pure enantiomer).

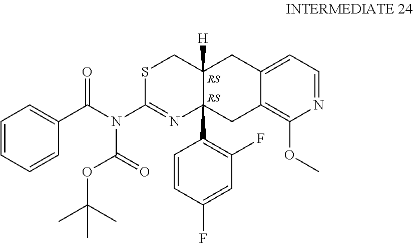 [SMILES: COc1nccc2c1C[C@]1(c3ccc(F)cc3F)N=C(N(C(=O)OC(C)(C)C)C(=O)c3ccccc3)SC[C@@H]1C2]

Intermediate 23 (2.2 g, 4.73 mmol) was dissolved in THF (75 mL) before di-tert-butyl dicarbonate (2.06 g, 9.45 mmol) was added, followed by 4-dimethylaminopyridine (173.21 mg, 1.42 mmol). The r.m. was stirred at r.t. for 30 min, it was then diluted with 40 mL of water and the material was acidified using 1M HCl. The organic material was then extracted using DCM and the organic layers were dried over MgSO₄ before being filtered and concentrated by evaporation. The product was purified by column chromatography (silica; MeOH/DCM 0/100 to 1/99) and the fractions containing product were combined and concentrated by evaporation to yield intermediate 24 (2.74 g).

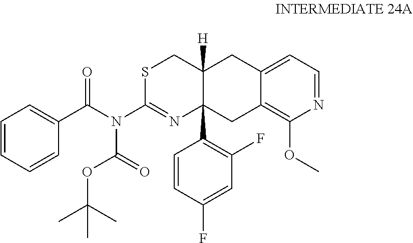 [SMILES: COc1nccc2c1C[C@]1(c3ccc(F)cc3F)N=C(N(C(=O)OC(C)(C)C)C(=O)c3ccccc3)SC[C@@H]1C2]

A stirred mixture of intermediate 23a (2.2 g, 0.0047 mol) in THF (20 mL, 0.89 g/mL, 0.25 mol) was treated with BOC-anhydride (1.24 g, 0.0057 mol) and DMAP (50 mg, 0.00041 mol). After stirring for 1 h at r.t., the r.m. was diluted with saturated NaHCO₃ solution (20 mL), water (50 mL) and EtOAc (100 mL) and the layers were separated. The aqueous layer was extracted with EtOAc (50 mL). The combined organic layers were treated with brine (20 mL), dried over MgSO₄, filtered and concentrated in vacuo to give intermediate 24a as a white foam (2.77 g, 99%).

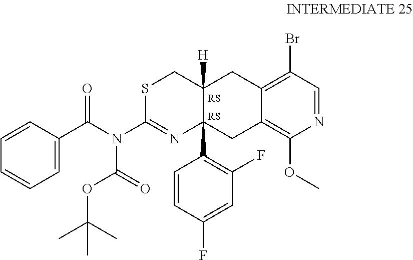 [SMILES: COc1ncc(Br)c2c1C[C@]1(c3ccc(F)cc3F)N=C(N(C(=O)OC(C)(C)C)C(=O)c3ccccc3)SC[C@@H]1C2]

Intermediate 24 (2.74 g, 4.84 mmol) was dissolved in ACN (80 mL) before N-bromosuccinimide (1.12 g, 6.30 mmol) was added in small portions at r.t. and the r.m. was then stirred for 22 h (overnight was required). The r.m. was then quenched with K₂CO₃ and stirred for 10 min before the organic material was extracted using DCM; the OL was then dried over MgSO₄, filtered and concentrated by evaporation to yield intermediate 25 (3.1 g, LCMS showed 18% of BOC-deprotected product after work-up).

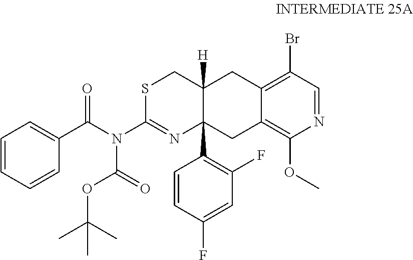 [SMILES: COc1ncc(Br)c2c1C[C@]1(c3ccc(F)cc3F)N=C(N(C(=O)OC(C)(C)C)C(=O)c3ccccc3)SC[C@@H]1C2]

To a stirred mixture of intermediate 24a (2.77 g, 0.0049 mol) in ACN (250 mL, 0.79 g/mL, 4.81 mol) was added N-bromosuccinimide (1 g, 0.0056 mol) in small portions and the ensuing r.m. was stirred for 4 days at r.t. then more N-bromosuccinimide (0.2 g, 0.0011 mol) was added and stirring was continued for another 3 h. The r.m. was diluted with 40 mL of saturated NaHCO₃, water (0.1 L), EtOAc (200 mL) and the layers were separated. The aqueous layer was extracted with EtOAc (50 mL) and the combined organic layers were treated with brine (0.1 L), dried over MgSO₄, filtered and concentrated in vacuo to afford an off white solid. This was purified by silica gel column chromatography using a 120 g Redisep flash column eluting with a gradient of 0-50% EtOAc in heptane to afford intermediate 25a as a bright white solid (2.1 g, yield 67%).

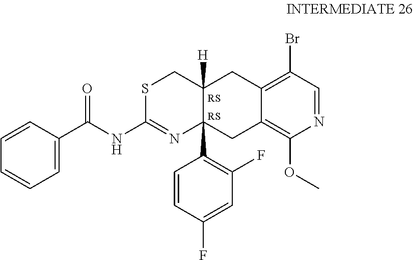 [SMILES: COc1ncc(Br)c2c1C[C@]1(c3ccc(F)cc3F)N=C(NC(=O)c3ccccc3)SC[C@@H]1C2]

Intermediate 25 (3.44 g, 5.34 mmol) and formic acid (20 mL, 530.14 mmol) were stirred at r.t. for one h. The r.m. was concentrated in vacuo and the crude was basified with sat. aq. Na₂CO₃ sol. The r.m. was extracted with DCM and the organic layer was dried over MgSO₄, filtered and concentrated in vacuo to afford intermediate 26 (2.9 g, 99%, cis).

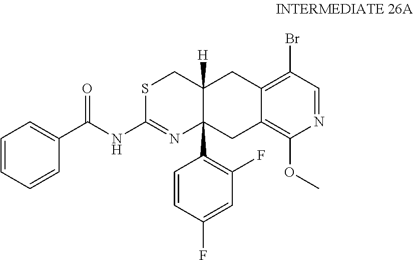 [SMILES: COc1ncc(Br)c2c1C[C@]1(c3ccc(F)cc3F)N=C(NC(=O)c3ccccc3)SC[C@@H]1C2]

Intermediate 25a (13.71 g, 21 mmol) and formic acid (79.7 mL, 2.1 mmol) were stirred at r.t. for 1 h. The formic acid present in the r.m. was evaporated and the product was basified with Na₂CO₃ before being extracted with DCM. The organic layer was dried over MgSO₄, filtered and concentrated by evaporation to yield a product that was crystallized from DIPE. The crystals were filtered off and dried, yielding intermediate 26a (9.32 g, 81%).

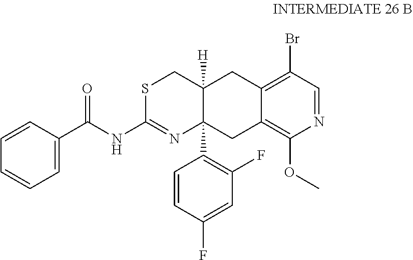 [SMILES: COc1ncc(Br)c2c1C[C@@]1(c3ccc(F)cc3F)N=C(NC(=O)c3ccccc3)SC[C@H]1C2]

Following a synthetic sequence similar to the one used for the synthesis of (in the order) intermediate 23a, 24a, 25a and 26a, intermediate 26b was obtained starting from intermediate 23b.

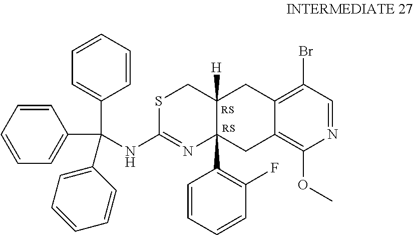 [SMILES: COc1ncc(Br)c2c1C[C@]1(c3ccccc3F)N=C(NC(c3ccccc3)(c3ccccc3)c3ccccc3)SC[C@@H]1C2]

Intermediate 27 was prepared following a synthetic procedure similar to the one reported for the synthesis of intermediate 18. Starting from compound 16 (0.47 g, 1.11 mmol) intermediate 27 was obtained (0.396 g, 54%, cis).

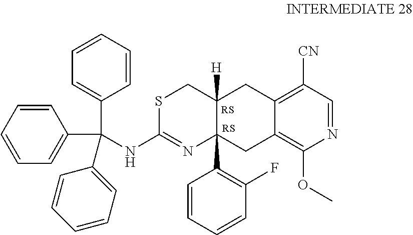 [SMILES: COc1ncc(C#N)c2c1C[C@]1(c3ccccc3F)N=C(NC(c3ccccc3)(c3ccccc3)c3ccccc3)SC[C@@H]1C2]

A microwave tube was charged with tris(dibenzylideneacetone)dipalladium(0) (25 mg, 0.027 mmol), 1,1′-bis(diphenylphosphino)ferrocene (30 mg, 0.053 mmol) in dimethylacetamide (10 mL) and degassed with nitrogen, then intermediate 27 (150 mg, 0.226 mmol), zinc dust (5 mg, 0.076 mmol) and zinc cyanide (108 mg, 0.9 mmol) were added. The tube was capped and heated at 120° C. for 12 h. The r.m. was allowed to cool down, poured onto ice water (30 mL) and extracted with EtOAc (2×20 mL). The combined organic layers were treated with water (2×5 mL), dried over MgSO₄, filtered and concentrated in vacuo to give a brown solid. This solid was purified by column chromatography (silica, MeOH/DCM 0/100 to 1/99) to afford intermediate 28 as a white solid (0.159 g, quantitative, cis), which was used as such in the next step.

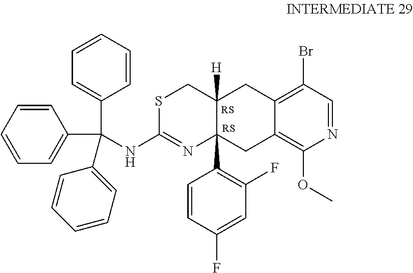 [SMILES: COc1ncc(Br)c2c1C[C@]1(c3ccc(F)cc3F)N=C(NC(c3ccccc3)(c3ccccc3)c3ccccc3)SC[C@@H]1C2]

Compound 17 (1.89 g, 4.29 mmol, obtained from intermediate 26 by Cbz cleavage with DBU as described in E4, second step) was dissolved in DMF (143.8 mL) before trimethylamine (1.19 mL, 8.6 mmol) was added, followed by triphenylmethyl chloride (3.59 g, 12.88 mmol). The r.m. was then heated to 80° C. for 18 h, then poured over −200 mL of ice water, which caused the precipitation of a brown solid. This was filtered off before being dissolved in DCM, dried over MgSO₄ and filtered. The water layer was washed 3 times with EtOAc and the combined organi layers were dried over MgSO₄ and filtered. All of the organic layers were combined and concentrated by evaporation to yield crude intermediate 29, which was purified by column chromatography (silica, MeOH/DCM 0/100 to 2/98). The pure fractions were combined and concentrated by evaporation and then crystallised in DIPE to yield intermediate 29 (1.42 g, 49%) as a white solid.

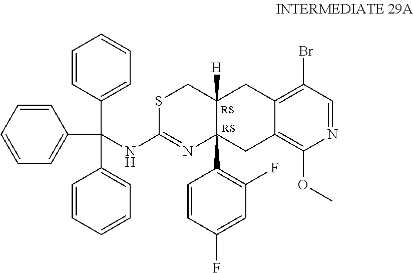 [SMILES: COc1ncc(Br)c2c1C[C@]1(c3ccc(F)cc3F)N=C(NC(c3ccccc3)(c3ccccc3)c3ccccc3)SC[C@@H]1C2]

Compound 40 (0.87 g, 1.976 mmol) was dissolved in dry ACN (76 mL) and then Et₃N (0.55 mL. 3.95 mmol) was added, followed by triphenylmethyl chloride (0.826 g, 2.96 mmol). The r.m. was then heated to 80° C. for 1.5 h, then the solvent was evaporated and the residue was dissolved in EtOAc. After basification of the mixture using K₂CO₃ the organic layer was washed with brine (×3) and the phases were separated. The combined organic layers were dried over MgSO₄, filtered and concentrated in vacuo. The crude product was purified by flash column chromatography (silica gel, EtOAc/heptane 0/100 to 10/90). The desired fractions were collected and evaporated in vacuo. The compound was triturated from MeOH and the crystals were filtered off and dried to yield intermediate 29a (1.3 g, 96%) as a white solid.

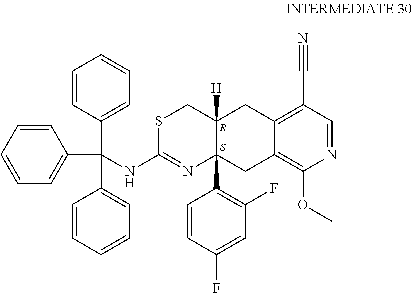

Tris(dibenzylideneacetone)dipalladium(0) (519.4 mg, 0.57 mmol) and 1,1′-bis(diphenylphosphino)ferrocene (649.7 mg, 1.17 mmol) were mixed in DMA (106.7 mL) in a mw vial and this mixture was degassed using nitrogen for 10 min. Intermediate 29a (1.6 g, 2.34 mol) was then added, followed by Zinc (183.9 mg, 2.81 mmol) and Zn(CN)₂ (2.20 g, 18.8 mmol). The vial was capped and heated for 4 h at 150° C. The r.m. was poured over ice water and stirred which caused the formation of a brown solid. The solid was filtered off, dissolved in DCM and washed with water. The organic layer was dried on MgSO₄, filtered and concentrated by evaporation. The product was purified by column chromatography (silica, MeOH/DCM 0/100 to 1/99). The pure fractions were combined and concentrated by evaporation to yield intermediate 30 (1.47 g, quantitative).

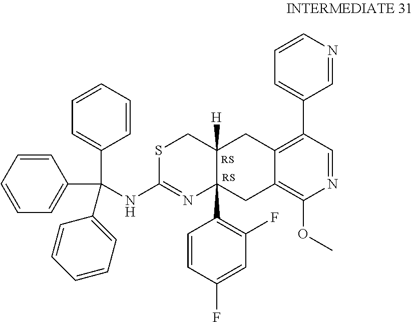 [SMILES: COc1ncc(-c2cccnc2)c2c1C[C@]1(c3ccc(F)cc3F)N=C(NC(c3ccccc3)(c3ccccc3)c3ccccc3)SC[C@@H]1C2]

1,1′-Bis(diphenylphosphino)ferrocene-palladium(II)dichloride dichloromethane complex (179.5 mg, 0.22 mmol) was added to a nitrogen degassed sol. of intermediate 29 (150 mg, 0.22 mmol), 3-pyridineboronic acid pinacol ester (54 mg, 0.26 mmol) and potassium carbonate (60.7 mg, 0.44 mmol) in 1,4-dioxane (8 mL) and water (2 mL) in a microwave vial, which was capped and heated at 100° C. for 17 h. The r.m. was extracted with DCM, and the organic layers were washed with brine, dried over MgSO₄, filtered and concentrated in vacuo. The product was purified by column chromatography (silica, MeOH/DCM 0/100 to 4/96) to afford intermediate 31 (60 mg, 40%, cis).

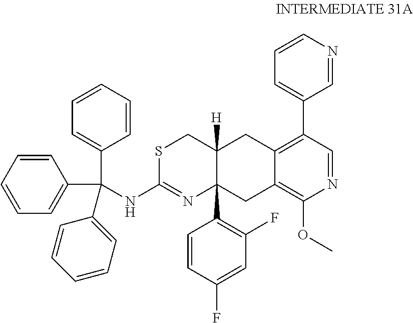 [SMILES: COc1ncc(-c2cccnc2)c2c1C[C@]1(c3ccc(F)cc3F)N=C(NC(c3ccccc3)(c3ccccc3)c3ccccc3)SC[C@@H]1C2]

1,1′-Bis(diphenylphosphino)ferrocene-palladium(II)dichloride dichloromethane complex (300 mg, 0.37 mmol) was added to a N₂ degassed solution of intermediate 29a (250 mg, 0.37 mmol), 3-pyridineboronic acid pinacol ester (90.1 mg, 0.44 mmol) and K₂CO₃ (101.2 mg, 0.73 mmol) in 1,4-dioxane (13.3 mL) and distilled water (3.3 mL) in a microwave vial, which was capped and heated to 100° C. for 17 h. The organic material was extracted using DCM, and the organic layers were washed with brine; this organic material was then dried over MgSO₄, filtered and concentrated by evaporation. The product was purified by column chromatography (silica, MeOH/DCM 0/100 to 4/96). The fractions containing product were combined and concentrated by evaporation to yield intermediate 31a (200 mg, 80%).

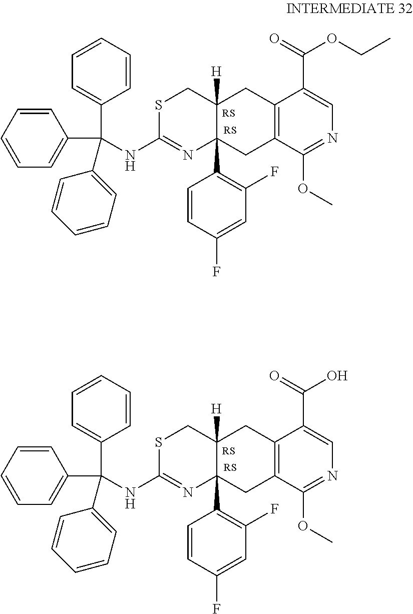 [SMILES: CCOC(=O)c1cnc(OC)c2c1C[C@H]1CSC(NC(c3ccccc3)(c3ccccc3)c3ccccc3)=N[C@@]1(c1ccc(F)cc1F)C2 ; COc1ncc(C(=O)O)c2c1C[C@]1(c3ccc(F)cc3F)N=C(NC(c3ccccc3)(c3ccccc3)c3ccccc3)SC[C@@H]1C2]

A 75 mL stainless steel autoclave was charged under nitrogen atmosphere with intermediate 29 (160 mg, 0.23 mmol), Pd(OAc)₂ (1 mg, 0.005 mmol), 1,3-bis(diphenylphosphino)propane (3.9 mg, 0.009 mmol), potassium acetate (46 mg, 0.469 mmol), THF (20 mL) and EtOH (20 mL). The autoclave was closed and pressurized to 30 bar CO. The r.m. was stirred at 120° C. for 16 h, then the solvents were evaporated, water was added and the product was extracted with DCM. The organic layer was dried over MgSO₄, filtered and evaporated in vacuo. The crude was purified by column chromatography (silica, EtOH/DCM 0/100 to 2/98) to afford intermediate 32 as a mixture of the ester and the acid, which was used as such in the next step (cis).

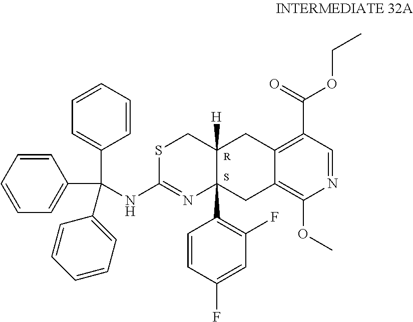

A 75 mL stainless steel autoclave was charged under nitrogen atmosphere with intermediate 29a (1.1 g, 1.61 mmol), Pd(OAc)₂ (77.2 mg, 0.34 mmol), 1,3-bis(diphenylphosphino)propane (44 mg, 0.107 mmol), potassium acetate (385 mg, 3.93 mmol), THF (20 mL) and EtOH (20 mL). The autoclave was closed and pressurized to 30 bar CO. The r.m. was stirred for 19 h at 120° C. The r.m. was evaporated before water and DCM were added and the mixture was filtered over Dicalite®. The organic material was extracted with DCM, dried over MgSO₄, filtered and concentrated by evaporation to yield 1.48 g or product, which was purified by column chromatography (silica, MeOH/DCM 0/100 to 2/98) and the fractions containing product were combined and concentrated by evaporation to yield intermediate 32a (0.902 g, 83%).

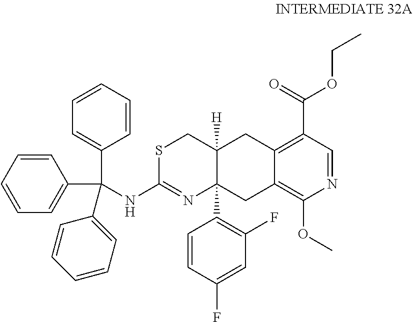 [SMILES: CCOC(=O)c1cnc(OC)c2c1C[C@@H]1CSC(NC(c3ccccc3)(c3ccccc3)c3ccccc3)=N[C@]1(c1ccc(F)cc1F)C2]

Following a synthetic sequence similar to the one used for the synthesis of (in the order) intermediate 26a, example E25, intermediate 29a and intermediate 32a, intermediate 32b was prepared starting from intermediate 26b (35% over three steps).

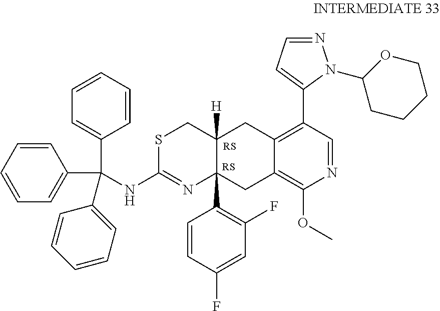

Intermediate 33 was prepared following a synthetic procedure similar to the one reported for the synthesis of intermediate 31. Starting from commercially available 1-(2-tetrahydropyranyl)-1H-pyrazole-5-boronic acid pinacol ester (147 mg, 0.53 mmol) and intermediate 29 (300 mg, 0.44 mmol) intermediate 33 was obtained (215 mg, 65%, cis).

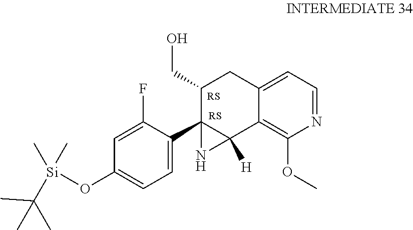

2-Bromo-4-[[(1,1-dimethylethyl)dimethylsilyl]oxy]-1-fluorobenzene (CAS 1037088-98-4, 9.327 g, 30.554 mmol) was stirred in THF (18.7 mL) under nitrogen atmosphere, then the mixture was cooled to −20° C. Isopropylmagnesium chloride (2 M in THF, 18.65 mL, 37.308 mmol) was added dropwise at −20° C. The r.m. was stirred at 0-5° C. for 1 h, then cooled to −40° C. Intermediate 4 (3.12 g, 15.277 mmol) was dissolved in THF (18.7 mL) and added dropwise to the r.m., which was then allowed to reach r.t. NH₄Cl sat. sol. was then added to quench the reaction, and the mixture was extracted with DCM. The organic layer was dried over MgSO₄, filtered and concentrated in vacuo. The residue was purified by flash chromatography (silica, MeOH/DCM 1/99 to 2/98). The desired fractions were collected and the solvent evaporated in vacuo to yield intermediate 34 as a yellowish foam (4.55 g, 69%, cis/trans).

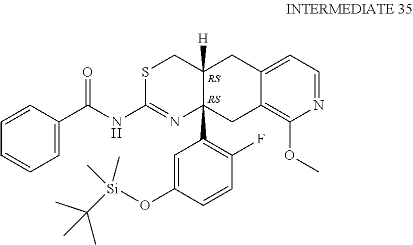 [SMILES: COc1nccc2c1C[C@]1(c3cc(O[Si](C)(C)C(C)(C)C)ccc3F)N=C(NC(=O)c3ccccc3)SC[C@@H]1C2]

Following a synthetic sequence similar to the one used for the synthesis of (in the order) intermediate 6, intermediate 7 and intermediate 8, intermediate 35 was prepared starting from intermediate 34 (66% over 3 steps, cis).

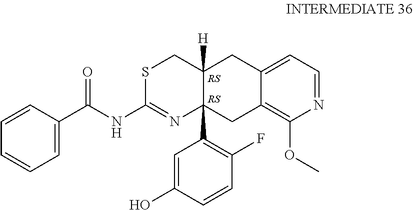 [SMILES: COc1nccc2c1C[C@]1(c3cc(O)ccc3F)N=C(NC(=O)c3ccccc3)SC[C@@H]1C2]

Tetrabutylammonium fluoride (1 M in THF, 2.42 mL, 2.42 mmol) was added dropwise to a sol. of intermediate 35 (1 g, 1.731 mmol) in THF (19.7 mL). The r.m. was strirred at r.t. for 40 min, then it was diluted with 100 mL of DCM, basified with NaHCO₃ sat. sol. maintaining the temperature below 5° C. ammonia in MeOH and extracted with DCM. The organic layer was separated, dried with MgSO₄, filtered and the solvent was evaporated in vacuo. The residue was purified by flash chromatography (silica, MeOH/DCM 0/100 to 1/99). The desired fractions were collected and the solvent evaporated to afford intermediate 36 (790 mg, 99%, cis).

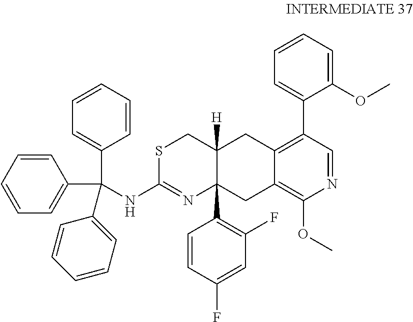 [SMILES: COc1ccccc1-c1cnc(OC)c2c1C[C@H]1CSC(NC(c3ccccc3)(c3ccccc3)c3ccccc3)=N[C@@]1(c1ccc(F)cc1F)C2]

Intermediate 37 was prepared following a synthetic sequence similar to the one used for the synthesis of intermediate 31a, starting from intermediate 29a and 2-methoxyphenylboronic acid.

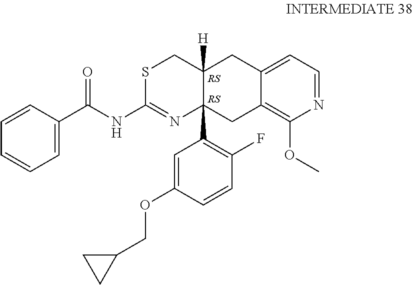 [SMILES: COc1nccc2c1C[C@]1(c3cc(OCC4CC4)ccc3F)N=C(NC(=O)c3ccccc3)SC[C@@H]1C2]

Intermediate 36 (260 mg, 0.56 mmol) was stirred in MeOH (18 mL). Na₂CO₃ (178 mg, 1.68 mmol) and (bromomethyl)cyclopropane (606 mg, 4.49 mmol) were added, and the reaction was stirred at 40° C. for 120 h. After this time the r.m. was concentrated in vacuo, then the residue was dissolved in DCM, the organic layer washed with water, dried over MgSO₄ and concentrated in vacuo. The crude material was purified by flash chromatography (silica, MeOH/DCM 0/100 to 1/99) to afford intermediate 38 (130 mg, 45%, cis).

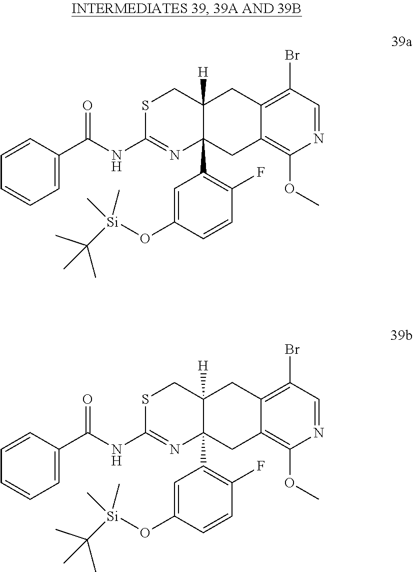 [SMILES: COc1ncc(Br)c2c1C[C@]1(c3cc(O[Si](C)(C)C(C)(C)C)ccc3F)N=C(NC(=O)c3ccccc3)SC[C@@H]1C2 ; COc1ncc(Br)c2c1C[C@@]1(c3cc(O[Si](C)(C)C(C)(C)C)ccc3F)N=C(NC(=O)c3ccccc3)SC[C@H]1C2]

Intermediate 39a was prepared following a synthetic procedure similar to the one reported for the synthesis of intermediate 15 (from intermediate 12). Starting from intermediate 35 (three batches of 2.41 g, 1 g and 5.15 g each with reaction times ranging from 1 to 4 h, were combined for purification), intermediate 39 was obtained, which was then separated by preparative SFC (Stationary phase: Kromasil (R,R) Whelk-O 1 (25×250 mm), Mobile phase: CO₂, iPrOH with 0.4% iPrNH₂) to afford desired intermediate 39a (942 mg, 16%) and intermediate 39b (914 mg, 16%).

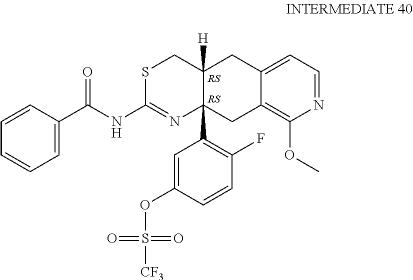 [SMILES: COc1nccc2c1C[C@]1(c3cc(OS(=O)(=O)C(F)(F)F)ccc3F)N=C(NC(=O)c3ccccc3)SC[C@@H]1C2]

Intermediate 36 (460 mg, 0.992 mmol) was stirred in DCM (19 mL) under nitrogen atmosphere. DIPEA (641 mg, 4.962 mmol) was added, and the r.m. was cooled to 0° C. Triflic anhydride (1 M in DCM, 1.7 ml, 1.7 mmol) was added dropwise. The r.m. was then allowed to reach r.t. and stirred for 1 h. After LC-MS control, additional 0.6 mL of triflic anhydride were added, and the r.m. allowed to stir for 1 h at r.t. 40 mL of DCM were then added and the r.m. cooled to 5° C. 10 mL of water were added dropwise and the r.m. was stirred for 10 min at r.t. The phases were separated, the organic layer dried over MgSO₄, filtered and concentrated in vacuo. The residue was purified by flash chromatography (silica, DCM) to afford intermediate 40 (520 mg, 88%, cis).

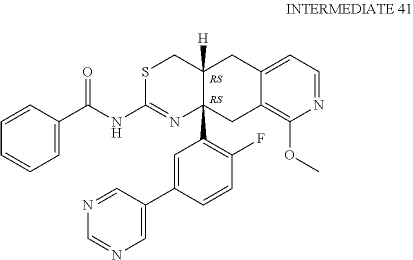 [SMILES: COc1nccc2c1C[C@]1(c3cc(-c4cncnc4)ccc3F)N=C(NC(=O)c3ccccc3)SC[C@@H]1C2]

Intermediate 40 (200 mg, 0.336 mmol) was stirred in 1,2-dimethoxyethane (2 mL). Pyrimidine-5-boronic acid (54 mg, 0.437 mmol), Pd(OAc)₂ (15 mg, 0.0672 mmol), 1,3-bis(diphenylphosphino)propane (42 mg, 0.10 mmol) and Na₂CO₃ sol. (2 M, 1 mL) were added. The r.m. was stirred at 90° C. for 4 h, then cooled to r.t. and extracted with DCM. The organic layer was dried over MgSO₄, filtered and concentrated in vacuo. Two subsequent purifications by flash chromatography (silica, MeOH/DCM 0/100 to 1/99, then EtOAc/DCM 10/90 to 30/70) afforded intermediate 41 (205 mg, 77%, cis).

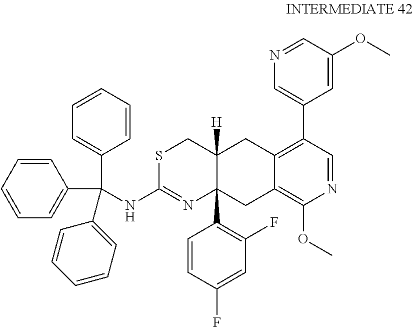 [SMILES: COc1cncc(-c2cnc(OC)c3c2C[C@H]2CSC(NC(c4ccccc4)(c4ccccc4)c4ccccc4)=N[C@@]2(c2ccc(F)cc2F)C3)c1]

Intermediate 42 was prepared following a synthetic procedure similar to the one reported for the synthesis of intermediate 31a, starting from intermediate 29a and 5-methoxypyridine-3-boronic acid (77 mg, 99% yield).

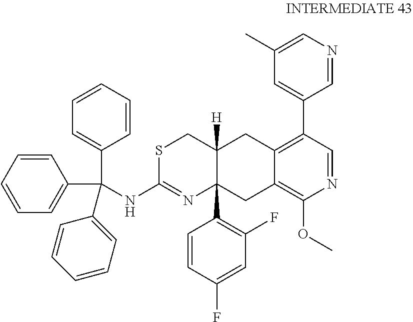 [SMILES: COc1ncc(-c2cncc(C)c2)c2c1C[C@]1(c3ccc(F)cc3F)N=C(NC(c3ccccc3)(c3ccccc3)c3ccccc3)SC[C@@H]1C2]

Intermediate 43 was prepared following a synthetic procedure similar to the one reported for the synthesis of intermediate 31a, starting from intermediate 29a and 5-methylpyridine-3-boronic acid (100 mg, 38% LC-MS purity).

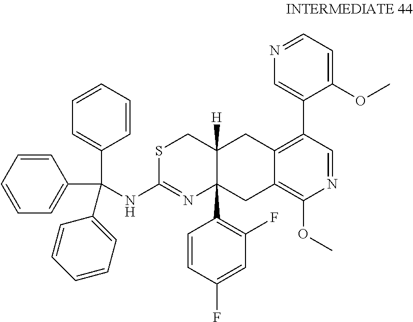 [SMILES: COc1ccncc1-c1cnc(OC)c2c1C[C@H]1CSC(NC(c3ccccc3)(c3ccccc3)c3ccccc3)=N[C@@]1(c1ccc(F)cc1F)C2]

Intermediate 44 was prepared following a synthetic procedure similar to the one reported for the synthesis of intermediate 31a, starting from intermediate 29a and 4-methoxy-3-(4,4,5,5-tetramethyl-1,3,2-dioxaborolan-2-yl)pyridine (280 mg, 37% LC-MS purity).

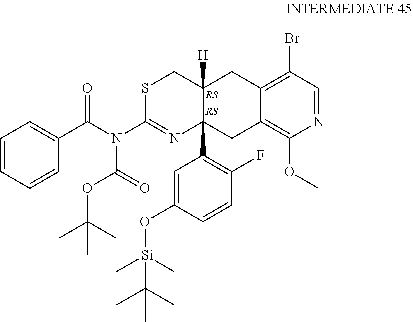 [SMILES: COc1ncc(Br)c2c1C[C@]1(c3cc(O[Si](C)(C)C(C)(C)C)ccc3F)N=C(N(C(=O)OC(C)(C)C)C(=O)c3ccccc3)SC[C@@H]1C2]

Following a synthetic sequence similar to the one used for the synthesis of (in the order) intermediate 13 and intermediate 14, intermediate 45 (cis) was obtained starting from intermediate 35 (40% over two steps, cis).

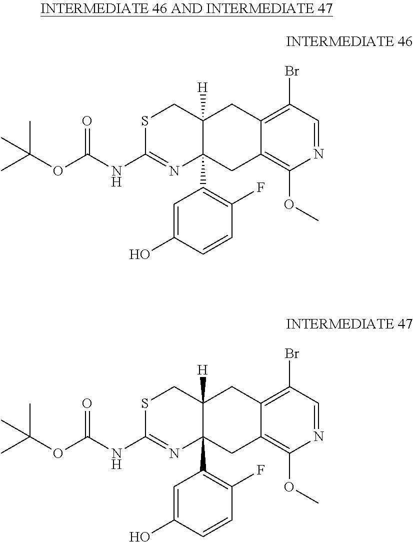

K₂CO₃ (5.75 g, 41.62 mmol) was added to a suspension of intermediate 45 (6.3 g, 8.32 mmol) in MeOH (67 mL), and the mixture was heated at 50° C. for 45 min, then it was concentrated in vacuo. EtOAc was added and the organic layer washed with NaHCO₃ aq. The organic layer was separated, dried over MgSO₄, filtered and concentrated in vacuo to affor a racemic mixture which was purified via Prep SFC (Stationary phase: Chiralpak Diacel AD 20×250 mm, Mobile phase: CO₂, iPrOH with 0.2% iPrNH₂) to afford intermediate 46 as a yellow solid (2.61 g, 58%) and intermediate 47 as a yellow solid (2.58 g, 58%).

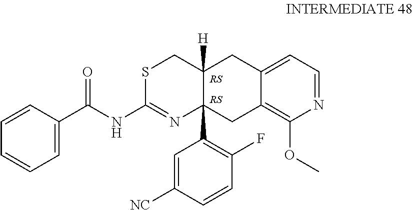 [SMILES: COc1nccc2c1C[C@]1(c3cc(C#N)ccc3F)N=C(NC(=O)c3ccccc3)SC[C@@H]1C2]

Zinc cyanide (14 mg, 0.118 mmol) was added to a stirred mixture of intermediate 40 (140 mg, 0.235 mmol) and Pd(PPh₃)₄ (14 mg, 0.012 mmol) in DMF (1 mL) under nitrogen. The mixture was heated at 120° C. for 20 min under microwave irradiation, then it mixed with another batch of material from a r.m. resulting from use of 54 mg of intermediate 40, the resulting mixture was diluted with EtOAc and the solid was filtered off through a celite pad. The filtrate was basified with sat. aq. Na₂CO₃. The organic layer was separated, dried over Na₂SO₄, filtered and the solvent was evaporated. The residue was purified by flash column chromatography (silica, EtOAc/heptane 0/100 to 30/70). The desired fractions were collected and the solvent evaporated in vacuo intermediate 48 (71 mg) as a white solid (cis).

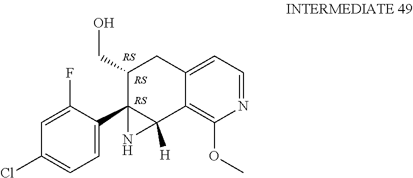 [SMILES: COc1nccc2c1[C@@H]1N[C@]1(c1ccc(Cl)cc1F)[C@H](CO)C2]

1-Bromo-4-chloro-2-fluorobenzene (20.51 g, 97.93 mmol) was stirred in THF under nitrogen and the r.m. cooled to −15° C. Isopropylmagnesium chloride (2 M in THF, 59.8 mL, 119.6 mmol) was added dropwise at −15° C. The r.m. was stirred at 0-5° C. for 1 h, then cooled to −15° C. Intermediate 4 (10 g, 48.97 mmol) dissolved in THF (total amount of THF 120 mL) was added dropwise and the mixture was allowed to reach r.t. NH₄Cl sat. sol. was then added dropwise and the r.m. extracted with DCM. The organic layer was dried over MgSO₄, filtered and concentrated in vacuo. The product was purified by flash column chromatography (silica, MeOH/DCM 0/100 to 4/96). The purest fractions were evaporated, yielding intermediate 49 (8.3 g, 51%).

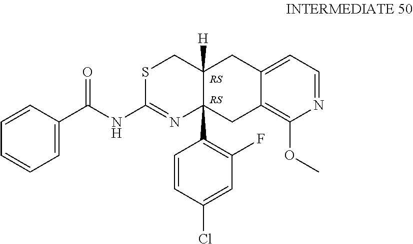 [SMILES: COc1nccc2c1C[C@]1(c3ccc(Cl)cc3F)N=C(NC(=O)c3ccccc3)SC[C@@H]1C2]

Following a synthetic sequence similar to the one used for the synthesis of (in the order) intermediate 6, intermediate 7 and intermediate 8, intermediate 50 (cis) was obtained starting from intermediate 49 (42% over three steps, cis).

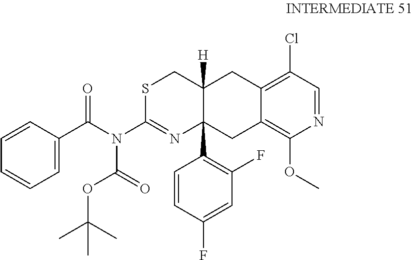 [SMILES: COc1ncc(Cl)c2c1C[C@]1(c3ccc(F)cc3F)N=C(N(C(=O)OC(C)(C)C)C(=O)c3ccccc3)SC[C@@H]1C2]

Intermediate 24a (380 mg, 0.672 mmol) was dissolved in dry MeCN (138 mL) in a microwave vial before N-chlorosuccinimide (137 mg, 1.031 mmol) was added in small portions and the tube was capped and heated at 80° C. for 8 h. The organic layer was diluted with DCM and washed with aq. K₂CO₃ sol., then dried over MgSO₄, filtered and concentrated in vacuo. The organic residue was purified by flash column chromatography (silica, DCM) to afford intermediate 51 (400 mg, the material also contains de-BOC product).

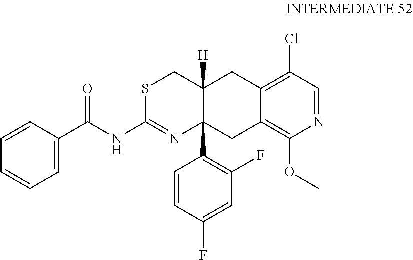 [SMILES: COc1ncc(Cl)c2c1C[C@]1(c3ccc(F)cc3F)N=C(NC(=O)c3ccccc3)SC[C@@H]1C2]

Following a synthetic procedure similar to the one used for the synthesis of intermediate 26a, intermediate 52 was obtained starting from intermediate 51 (200 mg, 60%).

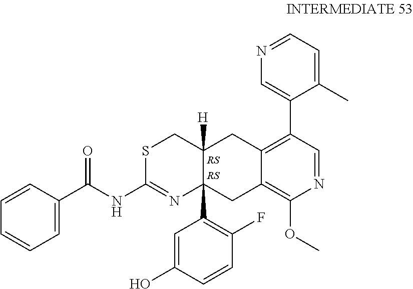 [SMILES: COc1ncc(-c2cnccc2C)c2c1C[C@]1(c3cc(O)ccc3F)N=C(NC(=O)c3ccccc3)SC[C@@H]1C2]

Following a synthetic procedure similar to the one used for the synthesis of intermediate 31 and using cesium fluoride instead of potassium carbonate, intermediate 57 was prepared starting from intermediate 39 (racemic) (200 mg, 79%, cis).

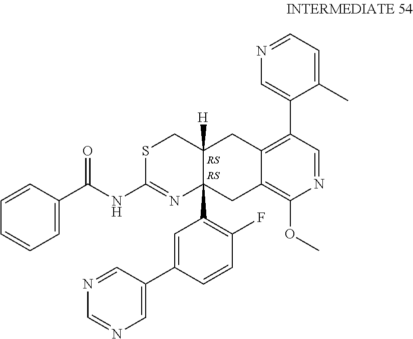 [SMILES: COc1ncc(-c2cnccc2C)c2c1C[C@]1(c3cc(-c4cncnc4)ccc3F)N=C(NC(=O)c3ccccc3)SC[C@@H]1C2]

Following a synthetic sequence similar to the one used for the synthesis of (in the order) intermediate 40 and intermediate 41, intermediate 54 was prepared starting from intermediate 53 (15%, cis).

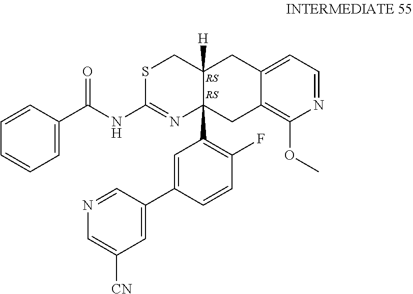 [SMILES: COc1nccc2c1C[C@]1(c3cc(-c4cncc(C#N)c4)ccc3F)N=C(NC(=O)c3ccccc3)SC[C@@H]1C2]

Following a synthetic procedure similar to the one used for the synthesis of intermediate 41, intermediate 55 was obtained starting from intermediate 40 (cis) (two batches of 420 mg and 100 mg were combined prior to purification by column chromatography) and 5-cyano-3-pyridinyl boronic acid (320 mg, 67%).

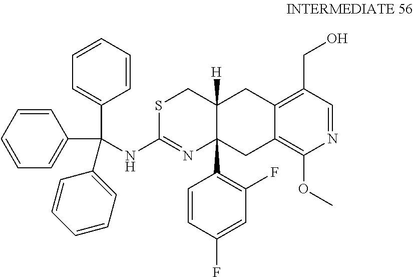 [SMILES: COc1ncc(CO)c2c1C[C@]1(c3ccc(F)cc3F)N=C(NC(c3ccccc3)(c3ccccc3)c3ccccc3)SC[C@@H]1C2]

Intermediate 32a (500 mg, 0.74 mmol) was dissolved in dry THF (83 mL) under nitrogen atmosphere. Lithium triethylborohydride (1 M, 3.7 mL, 3.7 mmol) was added dropwise at 0° C. and the r.m. was stirred overnight at r.t. MeOH was then added, followed by HCl 1 M (dropwise) until pH 4. DCM and water were subsequently added, the organic layer was separated, dried and the solvent was evaporated. The crude was used as such in the subsequent reaction step (440 mg).

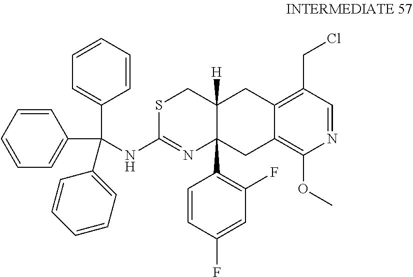 [SMILES: COc1ncc(CCl)c2c1C[C@]1(c3ccc(F)cc3F)N=C(NC(c3ccccc3)(c3ccccc3)c3ccccc3)SC[C@@H]1C2]

Intermediate 56 (340 mg, 0.54 mmol) was dissolved in dry DCM (10 mL) and DIPEA (0.185 mL, 1.07 mmol) was added. The r.m. was stirred while cooling with an ice-batch and methansulphonyl chloride (63 μL, 0.81 mmol) was added dropwise. The r.m. was then stirred at 0° C. for 3 h until LC-MS showed complete conversion to the desired product. NaHCO₃ aq. sol. was added and the organic layer was separated, dried over MgSO₄, filtered and the solvent was evaporated in vacuo. The crude was used as such in the subsequent reaction.

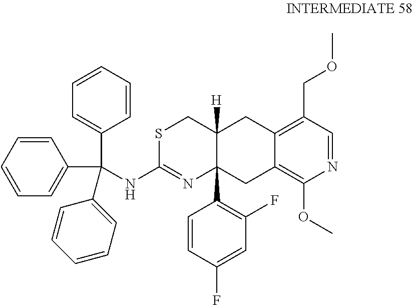 [SMILES: COCc1cnc(OC)c2c1C[C@H]1CSC(NC(c3ccccc3)(c3ccccc3)c3ccccc3)=N[C@@]1(c1ccc(F)cc1F)C2]

Intermediate 57 (340 mg, crude material) was dissolved in MeOH (22 mL). Sodium methoxide (282 mg, 5.21 mmol) was added and the r.m. stirred 4 h at 60° C., then allowed to reach r.t. DCM and water were added, the organic layer was separated, dried over MgSO₄, filtered and the solvent was evaporated in vacuo. The residue was purified by flash column chromatography (silica, EtOAc/heptane 0/100 to 50/50) to afford intermediate 58 (200 mg).

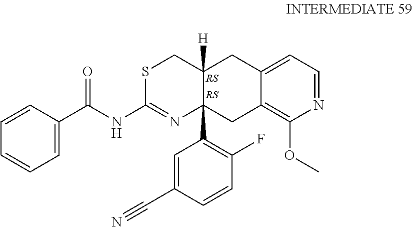 [SMILES: COc1nccc2c1C[C@]1(c3cc(C#N)ccc3F)N=C(NC(=O)c3ccccc3)SC[C@@H]1C2]

Zinc cyanide (47 mg, 0.392 mmol) was added to a stirred mixture of intermediate 40 (467 mg, 0.784 mmol) and Pd(PPh₃)₄ (45 mg, 0.039 mmol) in DMF (3.3 mL) under nitrogen. The r.m. was heated at 120° C. for 20 min under microwave irradiation, then it was diluted with EtOAc and the solid was filtered off through a celite pad. The filtrate was basified with aq. sat. Na₂CO₃, the organic layer was separated, dried over Na₂SO₄, filtered and the solvent evaporated. The residue was purified by flash column chromatography (silica, EtOAc/heptane 0/100 to 30/70) to afford intermediate 59 as a white solid (241 mg, 65%, cis).

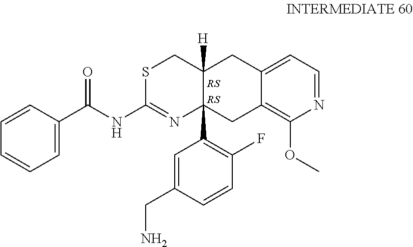 [SMILES: COc1nccc2c1C[C@]1(c3cc(CN)ccc3F)N=C(NC(=O)c3ccccc3)SC[C@@H]1C2]

Lithium aluminium hydride (2 M in THF, 0.21 mL, 0.42 mmol) was added dropwise to a solution of intermediate 59 (100 mg, 0.21 mmol) in THF (13.3 mL) under nitrogen cooled at −20° C. The solution was stirred at r.t. for 16 h, then cooled with an ice bath and quenched with 5% Rochelle salt solution. The mixture was extracted with DCM, the organic layer collected, dried over MgSO₄, filtered and concentrated in vacuo. The residue was purified by flash column chromatography (silica, 7 M ammonia in MeOH/DCM 0/100 to 1/99) to afford intermediate 60 as a colorless oil (58 mg, 78% purity, 45%, cis).

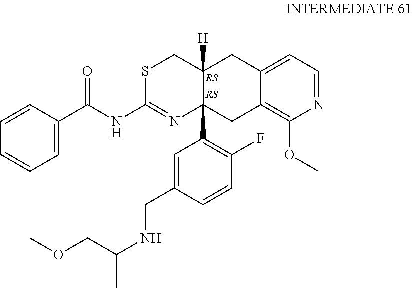 [SMILES: COCC(C)NCc1ccc(F)c([C@]23Cc4c(ccnc4OC)C[C@H]2CSC(NC(=O)c2ccccc2)=N3)c1]

To solution of intermediate 60 (58 mg, 78% purity) in dichloroethane (1.6 mL) and MeOH (2.7 mL) were added sodium acetate (13 mg, 0.16 mmol) and methoxyacetone (23 μL, 0.24 mmol). After stirring for 30 min sodium triacetoxyborohydride (52 mg, 0.24 mmol) was added. The r.m. was subsequently stirred for 30 min, then the residue was diluted with DCM. The organic layer washed with sat. aq. NaHCO₃ and with brine, dried over MgSO₄, filtered and the solvent evaporated in vacuo to give intermediate 61 as a colorless oil, used as such in the subsequent reaction (58 mg, cis).

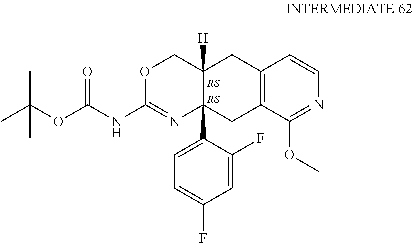 [SMILES: COc1nccc2c1C[C@]1(c3ccc(F)cc3F)N=C(NC(=O)OC(C)(C)C)OC[C@@H]1C2]

Boc anhydride (150 μL, 0.70 mmol) was added at rt to a solution of compound 30 (80 mg, 023 mmol) and DIPEA (200 μL, 1.16 mmol) in DCM (4 mL). The r.m. was stirred at r.t. overnight. Sat. NaHCO₃ sol. was added and the organic layer was separated, dried over MgSO₄ and filtered. The residue was purified by column chromatography (silica; flash purification system, gradient EtOAc/heptane from 0/100 to 90/10 step 20/80, 40/60, 60/40 and 80/20 12 g 20 min). The product fractions were collected and the solvent was evaporated to yield intermediate 62 (80 mg, 78%) as a colourless oil.

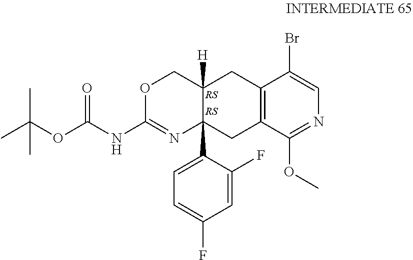 [SMILES: COc1ncc(Br)c2c1C[C@]1(c3ccc(F)cc3F)N=C(NC(=O)OC(C)(C)C)OC[C@@H]1C2]

Intermediate 62 (80 mg, 0.18 mmol) was dissolved in ACN (3 mL) before N-bromosuccinimide (48 mg, 0.27 mmol) was added in small portions at r.t.; the r.m. was then allowed to stir for 6 h at 60° C. (overnight at RT) and the reaction went to completion. The r.m. was then quenched with sat. sol. of NaHCO₃ and the organic material was extracted using DCM. The organic layer was then dried over MgSO₄, filtered and concentrated by evaporation. The residue was purified by column chromatography (silica; flash purification system, gradient n-heptane/EtOAc from 100/0 to 50/50 12 g 30 minutes). The product fractions were collected and the solvent was evaporated to yield intermediate 63 (25 mg, 27%) as a white solid.

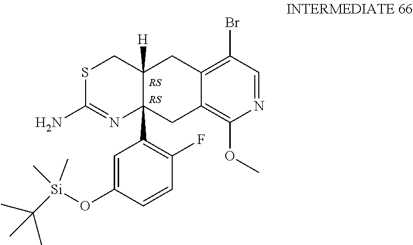 [SMILES: COc1ncc(Br)c2c1C[C@]1(c3cc(O[Si](C)(C)C(C)(C)C)ccc3F)N=C(N)SC[C@@H]1C2]

Hydrazine hydrate (46 μL, 0.761 mmol) was added to a mixture of racemic intermediate 39 (100 mg, 0.152 mmol) in EtOH (1.4 mL), to afford a suspension which turned into a solution after stirring at r.t. for 16 h. The r.m. was then evaporated and the crude purified by flash column chromatography (silica, 7 M ammonia in MeOH/DCM 0/100 to 1/99 to afford intermediate 69 (64 mg, 76%, cis) as a white solid.

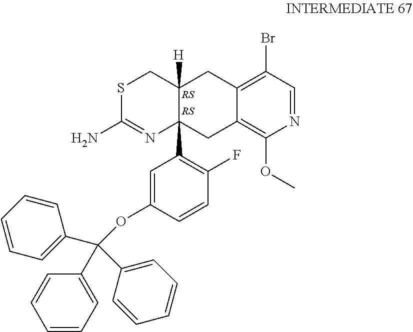 [SMILES: COc1ncc(Br)c2c1C[C@]1(c3cc(OC(c4ccccc4)(c4ccccc4)c4ccccc4)ccc3F)N=C(N)SC[C@@H]1C2]

Triethylamine (14 μL, 0.1 mmol) and triphenylmethyl chloride (37 mg, 0.133 mmol) were added to a solution of compound 18 (39 mg, 0.9 mmol) in dry MeCN (5 mL). The r.m. was heated to 80° C. for 3 h. Additional triphenylmethyl chloride (0.4 eq) was added and the r.m. heated to 80° C. for 1 h, then the solvent was evaporated, the organic residue dissolved in EtOAc and the r.m. basified with K₂CO₃. The organic layer was washed with brine (3×), dried over MgSO₄, filtered and concentrated in vacuo. The residue was purified by flash column chromatography (silica, EtOAc/heptane 0/100 to 10/90). The desired fractions were collected and the solvent evaporated to yield intermediate 70 as a white solid (22 mg, 37%, cis).

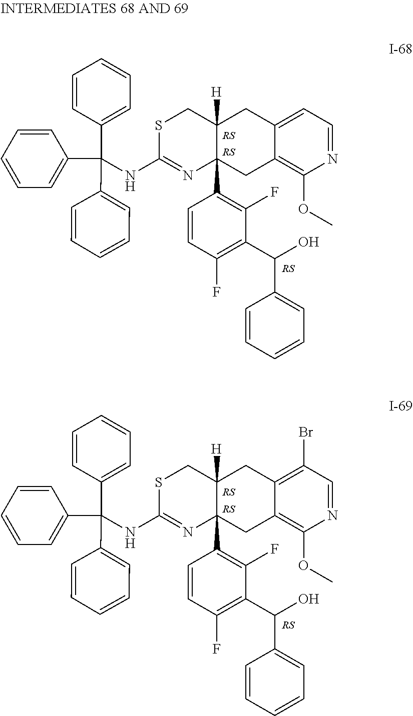 [SMILES: COc1nccc2c1C[C@]1(c3ccc(F)c(C(O)c4ccccc4)c3F)N=C(NC(c3ccccc3)(c3ccccc3)c3ccccc3)SC[C@@H]1C2 ; COc1ncc(Br)c2c1C[C@]1(c3ccc(F)c(C(O)c4ccccc4)c3F)N=C(NC(c3ccccc3)(c3ccccc3)c3ccccc3)SC[C@@H]1C2]

A solution of I-29 (400 mg, 0.59 mmol) in dry THF (11.4 mL) was cooled down to −78° C. BuLi (1.6 M in hexane, 0.84 mL, 1.35 mmol) was added dropwise and the mixture was stirred at −78° C. for 30 min. Then, benzaldehyde (0.18 mL, 1.76 mmol) was added dropwise and after 5 min at −78° C. the reaction was allowed to warm up to rt. The reaction was quenched with sat. NH₄Cl (10 mL) and diluted with EtOAc (20 mL). The organic phase was separated and the aqueous layer extracted with EtOAc (20 mL). The combined organic layers were dried over MgSO₄, filtered and evaporated. The crude was purified by flash chromatography on silica gel (24 g, heptane/EtOAc 100/0 to 70/30) to yield I-68 (155 mg, 34%) and I-69 (114 mg, 27%).

Intermediates I-70 to I-73 were prepared in an analogous manner from the indicated started material(s):

| | | Starting |
|---|---|---|
| Intermediate | | material(s) |
| 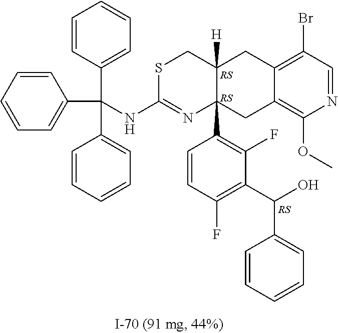 | 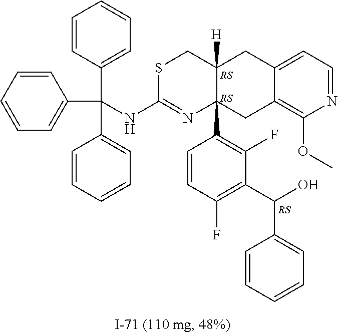 | I-29 (200 mg) |
| 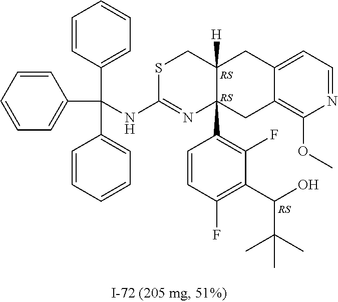 | 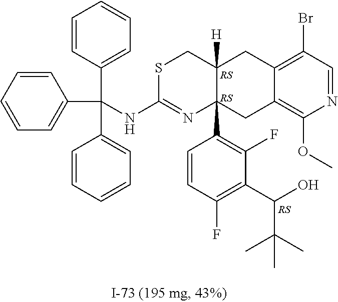 | I-29 (400 mg) 2,2-dimethyl- propanal [630- 19-3] (191 μL, 1.75 mmol) |

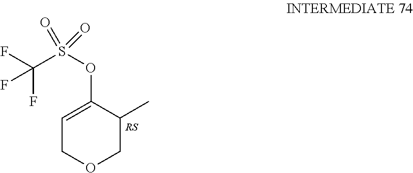

To a solution of tetrahydro-3-methyl-4H-pyran-4-one ([119124-53-7], 1 g, 8.76 mmol) in dry THF (40 mL) under N₂ and at −78° C. was added LiHMDS (1.0 M in THF, 9.64 mL, 9.64 mmol). After 45 min, 1,1,1-trifluoro-N-phenyl-N-[(trifluoromethyl)sulfonyl]-methanesulfonamide ([37595-74-7], 3.44 g, 9.64 mmol) was added dropwise as a sol. in THF (20 mL) and the reaction was allowed to slowly warm up to rt. The reaction was quenched with water (10 mL) and extracted with Et₂O (2×15 mL). The organic layers were combined, dried over MgSO₄ and concentrated in vacuo. The crude was purified by gel chromatography on silica gel (12 g, gradient: EtOAc/heptane 0/100 to 20/80). The product was isolated as a colorless oil contaminated with EtOAc (750 mg, 33% pure, 11% yield).

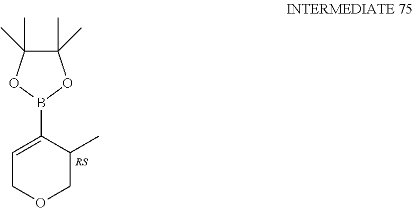

A 10 mL MW vial was charged with I-74 (250 mg, 1.02 mmol), bis(pinacolato)diboron ([73183-34-3], 386.78 mg, 1.52 mmol), [1,1′-bis(diphenylphosphino)ferrocene]dichloropalladium(II) (74.30 mg, 0.102 mmol) and KOAc (298.96 mg, 3.05 mmol). The vial was flushed with N₂, then dioxane was added and the reaction was stirred at 80° C. overnight. The reaction was cooled down and filtered through a pad of Celite®, washing with EtOAc. The crude was purified by flash chromatography on silica gel (gradient: EtOAc/heptane 0/100 to 10/90). The product was isolated contaminated with byproducts from the previous step and various amounts of heptane and EtOAc (155 mg).

### B. Preparation of the Final Compounds

### Example E1—Preparation of Compound 1

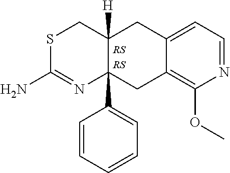 [SMILES: COc1nccc2c1C[C@]1(c3ccccc3)N=C(N)SC[C@@H]1C2]

A sol. of intermediate 8 (31.3 mg, 0.073 mmol) in MeOH (4 mL) was treated with DBU (80 μL, 0.54 mmol) and stirred at 70° C. for 16 h. The r.m. was then concentrated in vacuo. The oil was purified by flash chromatography (silica, 7 M ammonia in MeOH/DCM 0/100 to 10/90) to afford compound 1 as a white solid (15.7 mg, 66%, cis).

### Example E2—Preparation of Compound 2

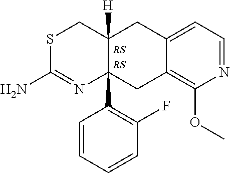 [SMILES: COc1nccc2c1C[C@]1(c3ccccc3F)N=C(N)SC[C@@H]1C2]

Compound 2 was prepared following a synthetic procedure similar to the one reported for the synthesis of compound 1. Starting from intermediate 12 (0.1 g, 0.22 mmol) compound 2 was obtained as a white solid (76.6 mg, 100%, cis).

### Example E3—Preparation of Compound 20

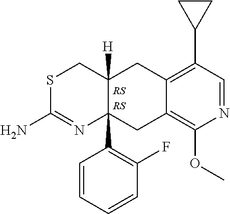 [SMILES: COc1ncc(C2CC2)c2c1C[C@]1(c3ccccc3F)N=C(N)SC[C@@H]1C2]

Compound 20 was prepared following a synthetic procedure similar to the one reported for the synthesis of compound 1. Starting from intermediate 16 (72 mg, 0.15 mmol) compound 20 was obtained as a white solid (12.2 mg, 21%, cis).

### Example E4—Preparation of Compound 16

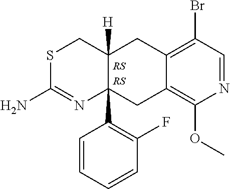 [SMILES: COc1ncc(Br)c2c1C[C@]1(c3ccccc3F)N=C(N)SC[C@@H]1C2]

(Step 1) To stirred mixture of intermediate 14 (0.15 g, 0.24 mol) in DCM (5 mL) was added TFA (1 mL, 13 mmol). After 10 min the r.m. was diluted with DCM (20 mL) and sat. aq. NaHCO₃ sol. until basic pH and the layers were separated. The organic layer was dried over MgSO₄, filtered and concentrated in vacuo to give an oil. (Step 2) This oil was dissolved in MeOH (10 mL) and treated with DBU (0.36 mL, 2.4 mmol). The r.m. was heated at 65° C. for 16 h, then the r.m. was concentrated in vacuo to afford an oil. This oil was purified by flash chromatography (silica, 7 M ammonia in MeOH/DCM 0/100 to 4/96) to afford an oil, which was triturated with Et₂O. The resulting white solid was dried (vacuum oven, 60° C., 1 h) to afford compound 16 (47 mg, 47%, cis).

### Example E5—Preparation of Compound 34

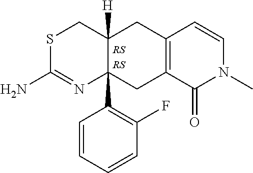 [SMILES: Cn1ccc2c(c1=O)C[C@]1(c3ccccc3F)N=C(N)SC[C@@H]1C2]

Compound 34 was prepared following a synthetic procedure similar to the one reported for the synthesis of compound 1. Starting from intermediate 17 (80.1 mg, 0.18 mmol) compound 34 was obtained as a white solid (20 mg, 33%, cis).

### Example E6—Preparation of Compound 3

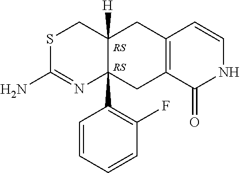 [SMILES: NC1=N[C@@]2(c3ccccc3F)Cc3c(cc[nH]c3=O)C[C@H]2CS1]

A microwave tube was charged with intermediate 19 (0.14 g, 0.24 mmol), MeOH (10 mL) and AcOH (10 mL, 17 mmol). The tube was capped and heated at 80° C. in a DrySyn metal heating block for 20 h. The r.m. was concentrated in vacuo to give a yellow oil. This oil was purified by preparative HPLC (Stationary phase: RP XBridge Prep C18 OBD-10 μm, 30×150 mm, Mobile phase: 0.25% NH₄HCO₃ sol. in water, MeOH) to afford compound 3 as a white solid (0.037 g, 46%, cis).

### Example E7—Preparation of Compound 35 and Compound 182

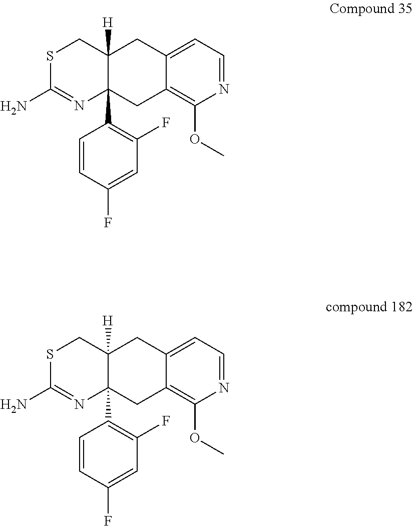 [SMILES: COc1nccc2c1C[C@]1(c3ccc(F)cc3F)N=C(N)SC[C@@H]1C2 ; COc1nccc2c1C[C@@]1(c3ccc(F)cc3F)N=C(N)SC[C@H]1C2]

A sol. of intermediate 23 (0.45 g, 0.97 mmol) in MeOH (50 mL) was treated with DBU (1.4 mL, 9.67 mmol) and stirred at 70° C. for 16 h in a closed vessel. The r.m. was then concentrated in vacuo. The oil was purified by flash chromatography (silica, 7 M ammonia in MeOH/DCM 0/100 to 5/95) to afford an oil. This residue was then purified by preparative SFC (Stationary phase: Chiralpak® AS, 20×250 mm, Mobile phase: CO₂, iPrOH with 0.2% iPrNH₂) to afford two fractions (two enantiomers). Each fraction was crystallized from DIPE to afford compound 35 (127 mg, 36%) and compound 183 (42 mg, 12%) as white solids.

### Example E8—Preparation of Compound 22

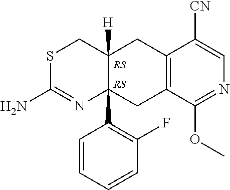 [SMILES: COc1ncc(C#N)c2c1C[C@]1(c3ccccc3F)N=C(N)SC[C@@H]1C2]

Compound 22 was prepared following a synthetic procedure similar to the one reported for the synthesis of compound 3. Starting from intermediate 28 (0.16 g, 0.262 mmol) compound 22 was obtained as a white solid (41.5 mg, 43%, cis).

### Example E9—Preparation of Compound 25

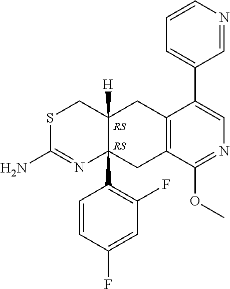 [SMILES: COc1ncc(-c2cccnc2)c2c1C[C@]1(c3ccc(F)cc3F)N=C(N)SC[C@@H]1C2]

Compound 25 was prepared following a synthetic procedure similar to the one reported for the synthesis of compound 3. Starting from intermediate 31 (60 mg, 0.088 mmol) compound 25 was obtained as a solid (20 mg, 52%, cis).

### Example E10—Preparation of Compound 24

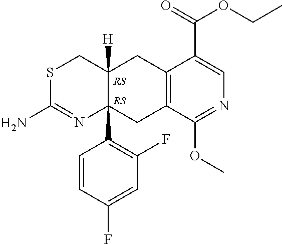 [SMILES: CCOC(=O)c1cnc(OC)c2c1C[C@H]1CSC(N)=N[C@@]1(c1ccc(F)cc1F)C2]

A microwave tube was charged with intermediate 32 (0.13 g, 0.19 mmol), EtOH (2 mL) and AcOH (60 mL). The tube was capped and heated at 80° C. for 24 h. The r.m. was concentrated in vacuo and the crude was diluted with water, DCM and NaHCO₃. The organic layer was dried over MgSO₄, filtered and evaporated in vacuo. The crude was purified by column chromatography (silica, EtOH/DCM 0/100 to 20/80). The pure fractions were collected, evaporated and the product was crystallized from Et₂O. The crystals were filtered off and dried to afford compound 24 (22 mg, 26%, cis).

### Example E11—Preparation of Compound 87

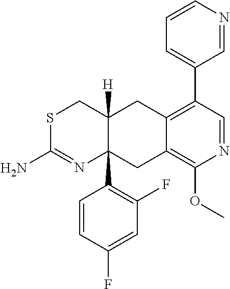 [SMILES: COc1ncc(-c2cccnc2)c2c1C[C@]1(c3ccc(F)cc3F)N=C(N)SC[C@@H]1C2]

Intermediate 31a (200 mg, 0.294 mmol) and TFA (5 mL) were stirred at 60° C. for 1 h. The r.m. was concentrated in vacuo and then neutralized with sat. aq. Na₂CO₃ sol. The r.m. was extracted with DCM and the organic layer was dried over MgSO₄, filtered and concentrated in vacuo. The crude was purified by flash chromatography (silica, MeOH/DCM 0/100 to 6/94) to afford compound 87 (79 mg, 61%) as an amorphous solid.

### Example E12—Preparation of Compound 29

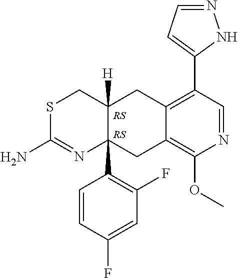 [SMILES: COc1ncc(-c2ccn[nH]2)c2c1C[C@]1(c3ccc(F)cc3F)N=C(N)SC[C@@H]1C2]

Intermediate 33 (215 mg, 0.285 mmol), AcOH (20 mL) and MeOH (20 mL) were placed in a pressure tube, which was capped and stirred at 80° C. for 17 h. The r.m. was concentrated in vacuo and then neutralized with Na₂CO₃. The mixture was extracted with DCM and the organic layer was dried over MgSO₄, filtered and concentrated in vacuo. The crude was purified by flash chromatography (silica, 7 M ammonia in MeOH/DCM 0/100 to 10/90). The fractions containing the product were combined and concentrated in vacuo to yield 25 mg of a mixture further purified by preparative SFC (Stationary phase: Chiralpak® Diacel AD, 30×250 mm, Mobile phase: CO₂, EtOH with 0.2% iPrNH₂), to afford compound 29 (7 mg, 6%, cis).

### Example E13—Preparation of Compounds 36 and 183

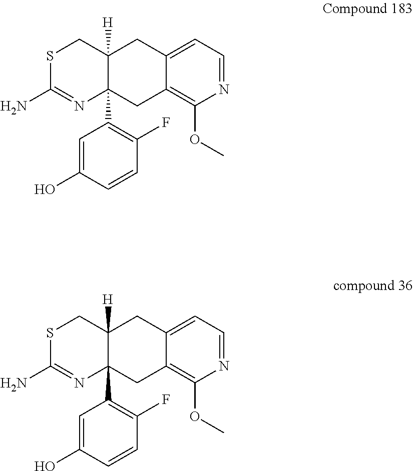 [SMILES: COc1nccc2c1C[C@@]1(c3cc(O)ccc3F)N=C(N)SC[C@H]1C2 ; COc1nccc2c1C[C@]1(c3cc(O)ccc3F)N=C(N)SC[C@@H]1C2]

Starting from intermediate 36, racemic compound 5 was obtained following a synthetic procedure similar to the one reported for the synthesis of compound 1. Compound 5 was separated into the single enantiomers by preparative SFC (Stationary phase: Chiralpak® Diacel AD, 20×250 mm, Mobile phase: CO₂, EtOH with 0.2% iPrNH₂), to afford compound 183 (9 mg, 23%) and compound 36 (7 mg, 18%).

### Example E14—Preparation of Compounds 184 and 136

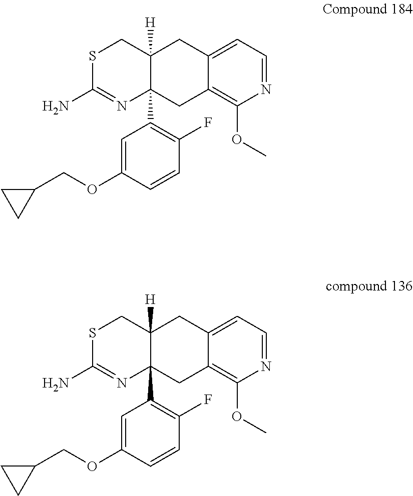 [SMILES: COc1nccc2c1C[C@@]1(c3cc(OCC4CC4)ccc3F)N=C(N)SC[C@H]1C2 ; COc1nccc2c1C[C@]1(c3cc(OCC4CC4)ccc3F)N=C(N)SC[C@@H]1C2]

Starting from intermediate 38, compound 14 was obtained as a racemic mixture following a synthetic procedure similar to the one reported for the synthesis of compound 1. The mixture was separated into the single enantiomers by preparative SFC (Stationary phase:Chiralpak® Diacel AD, 20×250 mm, Mobile phase: CO₂, EtOH with 0.2% iPrNH₂), to afford compound 184 (30 mg, 29%) and compound 136 (28 mg, 27%).

### Example E15—Preparation of Compound 106

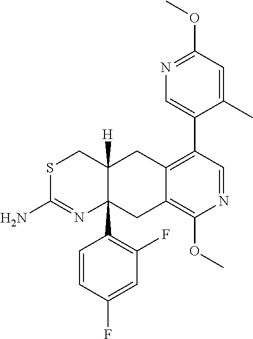 [SMILES: COc1cc(C)c(-c2cnc(OC)c3c2C[C@H]2CSC(N)=N[C@@]2(c2ccc(F)cc2F)C3)cn1]

1,1′-Bis(diphenylphosphino)ferrocene-palladium(II)dichloride dichloromethane complex (7 mg, 0.009 mmol) was added to a sol. of compound 40 (25 mg, 0.06 mmol), 2-methoxy-4-methylpyridine-5-boronic acid (28 mg, 0.17 mmol) and cesium fluoride (17 mg, 0.114 mmol) in 1,4-dioxane (2 mL) and water (0.5 mL) in a microwave vial under nitrogen atmosphere. The vial was capped and heated under microwave irradiation to 160° C. for 5 min, until LC-MS showed complete conversion to the desired product. The organic material was extracted using DCM, and the organic layers were washed with brine, dried over MgSO₄, filtered and concentrated in vacuo. The residue was purified by flash chromatography (silica, 7 M ammonia in MeOH/DCM 0/100 to 7.5/92.5) to afford compound 106 (9 mg, 33%).

### Example E16—Preparation of Compound 42

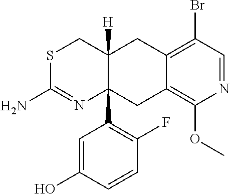 [SMILES: COc1ncc(Br)c2c1C[C@]1(c3cc(O)ccc3F)N=C(N)SC[C@@H]1C2]

Potassium carbonate (74 mg, 0.53 mmol) was added to a suspension of intermediate 39a (70 mg, 0.11 mmol) in MeOH (0.85 mL) and the mixture was heated at 65° C. for 4 h. The solvent was removed in vacuo, then EtOAc was added. The organic layer was washed with sat. NaHCO₃ sol., dried over MgSO₄ and solvent evaporated. The residual was purified by flash chromatography (silica, 7 M ammonia in MeOH/DCM 0/100 to 4/96) to afford compound 42 as a white solid (33 mg, 70%)

### Example E17—Preparation of Compound 108

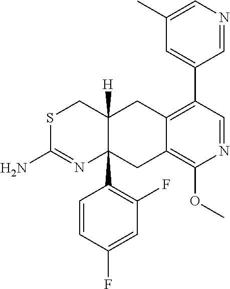 [SMILES: COc1ncc(-c2cncc(C)c2)c2c1C[C@]1(c3ccc(F)cc3F)N=C(N)SC[C@@H]1C2]

Starting from intermediate 43, a crude mixture containing compound 108 was obtained following a synthetic procedure similar to the one reported for the synthesis of compound 87. Purification by preparative HPLC (Stationary phase: RP XBridge Prep C18 OBD-10 μm, 30×150 mm, Mobile phase: 0.25% NH₄HCO₃ sol. in water, MeCN) afforded compound 108 (2 mg, 8%).

### Example E18—Preparation of Compound 103

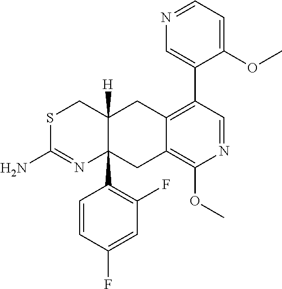 [SMILES: COc1ccncc1-c1cnc(OC)c2c1C[C@H]1CSC(N)=N[C@@]1(c1ccc(F)cc1F)C2]

Starting from intermediate 44, a crude mixture containing compound 103 was obtained following a synthetic procedure similar to the one reported for the synthesis of compound 87. Purification by preparative HPLC (Stationary phase: RP XBridge Prep C18 OBD-10 μm, 30×150 mm, Mobile phase: 0.25% NH₄HCO₃ sol. in water, MeCN) afforded, after coevaporation with MeOH (2×) and with DIPE, compound 103 (24 mg, 35%).

### Example E20—Preparation of Compound 27

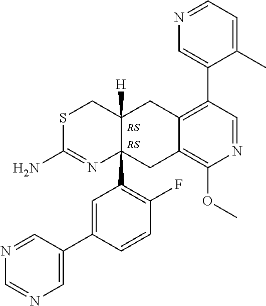 [SMILES: COc1ncc(-c2cnccc2C)c2c1C[C@]1(c3cc(-c4cncnc4)ccc3F)N=C(N)SC[C@@H]1C2]

Starting from intermediate 56, a crude mixture containing compound 27 was obtained following a synthetic procedure similar to the one reported for the synthesis of compound 1. Purification by Prep SFC (Stationary phase: Chiralpak Diacel AD, 20×250 mm, Mobile phase: CO₂, EtOH with 0.4% iPrNH₂) afforded compound 27 (6 mg, 26%, cis).

### Example E21—Preparation of Compounds 12, 37 and 186

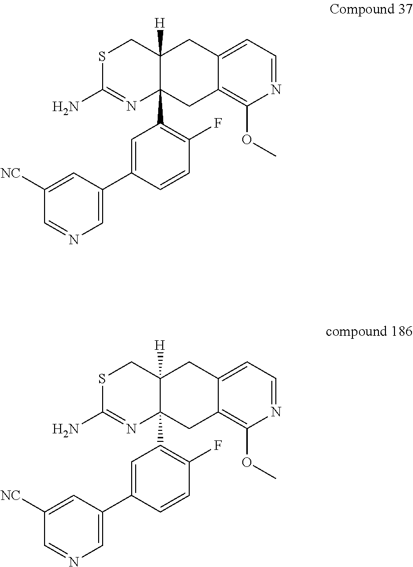 [SMILES: COc1nccc2c1C[C@]1(c3cc(-c4cncc(C#N)c4)ccc3F)N=C(N)SC[C@@H]1C2 ; COc1nccc2c1C[C@@]1(c3cc(-c4cncc(C#N)c4)ccc3F)N=C(N)SC[C@H]1C2]

Starting from intermediate 55, racemic compound 12 was obtained following a synthetic procedure similar to the one reported for the synthesis of compound 1. Purification by Prep SFC (Stationary phase: Chiralpak Diacel AD, 20×250 mm, Mobile phase: CO₂, iPrOH with 0.4% iPrNH₂) afforded compound 37 (47 mg, 22%) and compound 186 (50 mg, 24%).

### Example E22—Preparation of Compounds 10, 185 and 38

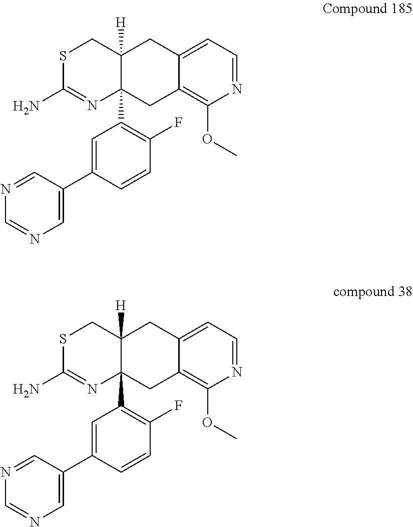 [SMILES: COc1nccc2c1C[C@@]1(c3cc(-c4cncnc4)ccc3F)N=C(N)SC[C@H]1C2 ; COc1nccc2c1C[C@]1(c3cc(-c4cncnc4)ccc3F)N=C(N)SC[C@@H]1C2]

Starting from intermediate 41, racemic compound 10 was obtained following a synthetic procedure similar to the one reported for the synthesis of compound 1. Purification by Prep SFC (Stationary phase: Chiralpak Diacel AD, 20×250 mm, Mobile phase: CO₂, EtOH with 0.4% iPrNH₂) afforded compound 185 (43 mg, 27%) and compound 38 (42 mg, 26%).

### Example E23—Preparation of Compound 30

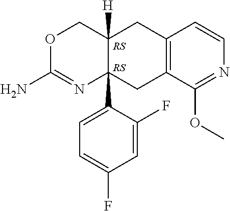 [SMILES: COc1nccc2c1C[C@]1(c3ccc(F)cc3F)N=C(N)OC[C@@H]1C2]

To a solution of intermediate 21 (650 mg, 2.03 mmol) in EtOH (12 mL) was added a solution of cyanogen bromide (540.9 mg, 5.11 mmol) in ACN (3 mL), and the mixture was stirred in a sealed tube at 85° C. for 5 h. The reaction was poured into NaHCO₃ solution and extracted with DCM, the organic layer was separated and dried with MgSO₄, filtered off and the solvent was evaporated under reduced pressure. The residue was purified by column chromatography (silica; flash purification system, gradient NH₃ in MeOH/DCM from 0/100 to 50/50 step at 10/90, 20/80, 30/70 and 35/65 12 g 25 min). The product fractions were collected and the solvent was evaporated to yield compound 30 (340 mg, 49%) as a white solid.

### Example E24—Preparation of Compound 31

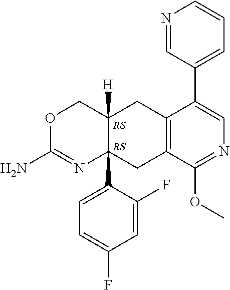 [SMILES: COc1ncc(-c2cccnc2)c2c1C[C@]1(c3ccc(F)cc3F)N=C(N)OC[C@@H]1C2]

Pd(dppf)Cl₂.CH₂Cl₂ (5.84 mg, 0.007 mmol) was added to a solution of intermediate 63 (25 mg, 0.048 mmol), pyridine-3-boronic acid (19 mg, 0.15 mmol) and CsF (15 mg, 0.10 mmol) in 1,4-dioxane (2 mL) and distilled water (0.5 mL) in a microwave vial under N₂ atmosphere, which was capped and heated under MW radiation to 160° C. for 5 min. The organic material was extracted using DCM, and the organic layers were washed with brine, dried over MgSO₄, filtered and concentrated by evaporation to yield compound 31 (10 mg, 50%).

### Example E25—Preparation of Compound 40

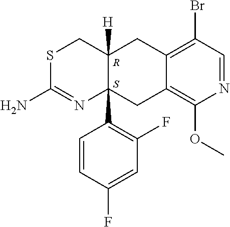

Intermediate 26a (9.32 g, 17 mmol), DBU (25.5 mL, 171 mmol) and MeOH (192.8 mL) were placed in a pressure tube and stirred at 60° C. overnight. The r.m. was concentrated by evaporation before the material was purified twice by column chromatography (silica, DCM to 5% MeOH in DCM). The fractions containing product were combined and concentrated by evaporation to yield compound 40 (6.84 g, 91%).

### Example E26—Preparation of Compound 202

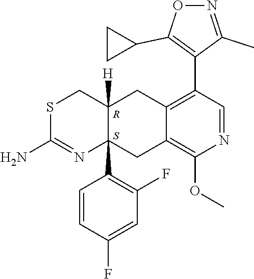

1,1′-Bis(diphenylphosphino)ferrocene-palladium(II) dichloride dichloromethane complex (27.82 mg, 0.034 mmol) was added to a solution of Co. No. 40, a mixture of 5-cyclopropyl-3-methyl-4-(4,4,5,5-tetramethyl-1,3,2-dioxaborolan-2-yl)-isoxazole [1628832-95-0] and 3-cyclopropyl-5-methyl-4-(4,4,5,5-tetramethyl-1,3,2-dioxaborolan-2-yl)-isoxazole [1628832-96-1] (178 mg, 0.715 mmol) and CsF (69 mg, 0.454 mmol) in 1,4-dioxane (5 mL) and distilled water (1.25 mL) in a microwave vial under N₂ atmosphere, which was capped and heated under microwave irradiation to 160° C. for 5 min. The organic material was extracted with DCM, and the organic layers were washed with brine, dried over MgSO4, filtered and concentrated by evaporation. The residue was purified by column chromatography (silica gel, NH₃ 7N MeOH in DCM 0/100 to 5/95). The desired fractions were collected, evaporated in vacuo and purified via Prep SFC (stationary phase: Chiralpak Diacel AD 20×250 mm, mobile: CO₂, EtOH+0.4 iPrNH₂) yielding 59 mg of a solid which was triturated with heptane to yield compound 202 (40 mg, 36%) as a yellow solid.

Compound 203 was prepared in an analogous manner from Co. No. 40:

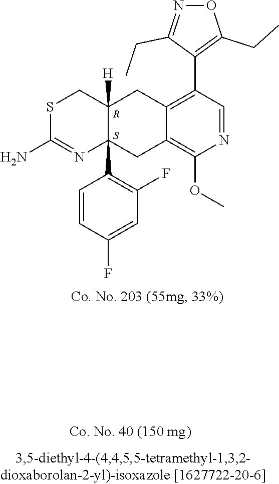

### Example E27—Preparation of Compound 204

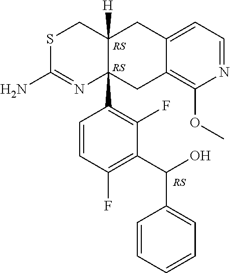

I-29 (114 mg, 0.161 mmol) in AcOH (2.5 mL, 43.67 mmol) and MeOH (2.5 mL) were heated in a MW vial to 80° C. overnight. The reaction mixture was concentrated under reduced pressure. The residue was partitioned between DCM (20 mL) and sat. Na₂CO₃ (20 mL). The organic phase was separated and the aqueous phase was extracted with DCM (20 mL, and 10 mL). The combined organic phases were then dried over MgSO₄, filtered and concentrated by evaporation. The crude was purified by chromatography on silica gel (12 g, gradient: from DCM 100% up to DCM/MeOH(NH₃) 97/3). Co. No. 204 was obtained as a white foamy solid (64 mg, 85%) as a mixture of two diastereomers.

Compound 205-206 were prepared in an analogous manner from the indicated starting material(s):

| Compound | Starting material |
|---|---|
| 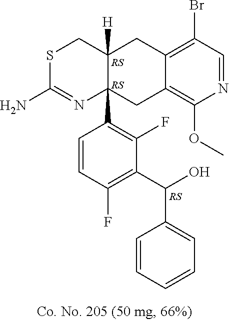 | I-70 (110 mg) |

| Compound | Starting material |
|---|---|
| 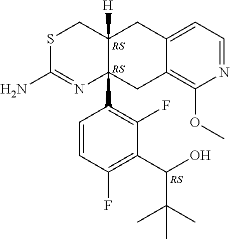 | I-72 (180 mg) |
| Co. No. 206 (40 mg, 34%) + | |
| additional fractions recovered | |
| containing separate or mixtures of | |
| diastereomers | |

### Example E28—Preparation of Compound 207

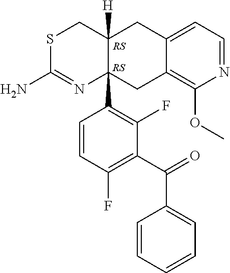

Co. No. 204 (64 mg, 0.137 mmol) was dissolved in DCM (3 mL) and Dess-Martin periodinane (87.09 mg, 0.205 mmol) was added. The reaction was stirred at rt for 3 h, then it was diluted with DCM (10 mL), NaHCO₃ sat. sol. (5 mL) and Na₂S₂O₃ sat. sol. (5 mL). The biphasic mixture was stirred vigorously for 10 min, then transferred into a separating funnel. The organic layer was separated and the aqueous layer was extracted with DCM (2×10 mL). The combined organic layers were dried over MgSO₄, filtered and evaporated. A purification was performed via Prep HPLC (Stationary phase: RP XBridge Prep C18 OBD-10 μm, 30×150 mm; mobile phase: 0.25% NH₄HCO₃ solution in water, MeOH) yielding Co. No. 207 (25 mg, 39%).

Compound 208 was prepared in an analogous manner from Co. No. 206:

| Compound | Starting material |
|---|---|
| 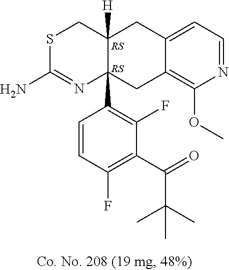 | Co. No. 206 (40 mg) |

### Example E29—Preparation of Compounds 209 and 210

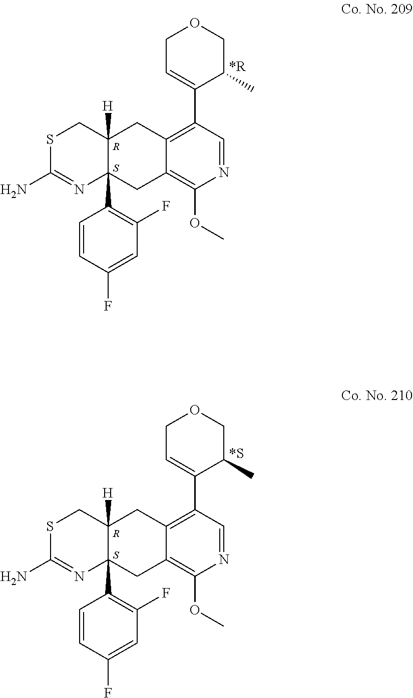

A 10 mL MW vial was charged with I-25a (50 mg, 0.0776 mmol), I-75 (43.463 mg, 0.194 mmol) and Pd(PPh₃)₄ (17.93 mg, 0.016 mmol) under a N₂ atmosphere. DME (0.6 mL) and sat. sol. NaHCO3 (0.2 mL) were added via syringe and the mixture was stirred at 120° C. for 3 h. The reaction was diluted with EtOAc (10 mL) and H₂O (5 mL). The organic phase was separated and the aqueous one extracted with EtOAc (10 mL). The combined organic layers were dried over MgSO₄, filetered and evaporated. The crude product was dissolved in MeOH, transferred into a closed vessel and treated with DBU at 80° C. for 2 h. LC-MS analysis showed formation of the desired product and Co. No. 35 as byproduct. The volatiles were removed in vacuo and the crude was submitted for purification by Prep HPLC (Stationary phase: RP XBridge Prep C18 ODB-5 μm, 30×250 mm; mobile phase: 0.25% NH₄HCO₃ solution in water, MeOH) Co. No. 209 (4 mg, 11%) and Co. No. 210 (3.5 mg, 10%).

Compounds 211-213 was prepared in an analogous manner from I-25a:

| Compound | Starting material |
|---|---|
| 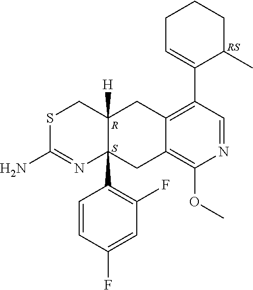 | I-25a (2 × 150 + 50 mg) |
| Co. No. 211 (125 mg, obtained from combination of three | |
| batches of reaction mixtures) was separated by Prep HPLC | |
| (stationary phase: RP XBridge Prep C18 ODB-5 μm, | |
| 30 × 250 mm; mobile phase: 0.25% NH₄HCO₃ solution in | |
| water, CH₃CN) into | |
| 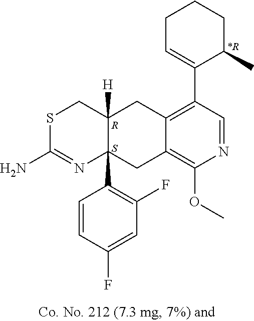 | |
| 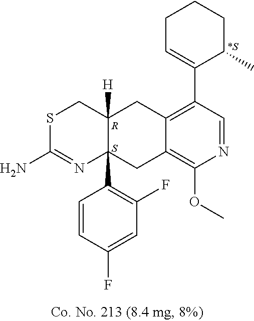 | |

### Example E30—Preparation of Compound 47

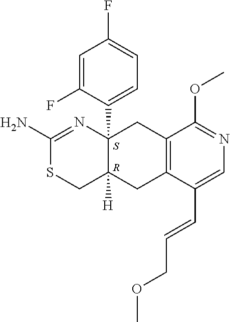

Co. No. 40 (50 mg) was mixed with potassium carbonate (47 mg, 3 eq) in water (250 μL), IPA (150 μL), THF (150 μL) and NMP (150 μL). Next (E)-2-(3-methoxyprop-1-enyl)-4,4,5,5-tetramethyl-1,3,2-dioxaborolane (22.49 mg, 1 eq) and dichloro[1,1′-bis(diphenylphosphino)ferrocene]palladium(II) acetone adduct (4.3 mg) were added. The reagents were mixed and heated at 150° C. for 30 min under mw irradiation. The crude material was poured into water and extracted with EtOAc. The organics were dried, filtered and evaporated to give a gum which was purified by reverse phase HPLC (solvent B—minimum 10%, intermediate 60%, maximum 95%) to give Co. No. 47 (10.4 mg, 21%) as a white solid.

Reference to RP HPLC in this example relates to purification using preparative HPLC. Fractions were lyophilised by freeze drying. The gradient profile was adjusted on a per sample basis to maximise resolution between the required compound and any intermediate. The preparative HPLC system consisted of the following components:

Gilson 322 pump with H₂ heads (0.3 to 30 ml/min)
Gilson 155 detector with semi-prep flow cell (0.5 mm pathlength)
Gilson 819 injector module
Gilson 506 system interface module
Gilson FC204 fraction collector set to take 100×16 mm tubes
Control was through Unipoint 5.11
HPLC Column: Phenomenex Luna, 5 μm C18 (2), 150 mm×21.2 mm
Solvent A: HPLC grade water containing 10 mM ammonium acetate (pH unadjusted)
Solvent B: HPLC grade acetonitrile

### Detection: 230 and 260 nm

Temperature: ambient

### Example E31—Preparation of Compounds According to General Procedure Flow Chemistry (Suzuki)

In one vessel was placed Co. No. 40 (20 mg) in NMP/IPA/THF (400 μL). In a second vessel, the boronic ester (3 eq.) and potassium carbonate (31.4 mg, 5 eq.) were dissolved in NMP/IPA/THF (400 μL). In a third vessel Pd(dppf)Cl₂ (1.6 mg, 0.05 eq.) was dissolved in THF (400 μL). The three vessels were loaded onto a Gilson 215 and injected into 250 μL injection loops and subsequently onto a 2 mL stainless steel coil heated to 150° C. with each pump running at 33 μL/min. The outflow injected automatically through a 20 μL loop into the purification (as described below) and assay part of the platform.

HPLC-MS was carried out using an Acquity™ Ultra Performance LC system, comprising a PDA detector, Binary Solvent Manager and SQ detector (Waters UK Ltd., Elstree, UK), tandem linked to a mass spectrometry system (Waters UK Ltd., Manchester, UK) employing vendor software (OpenLynx Browser™ v4.1, SQ Detector v4.1, Instrument Driver V4.1 and MassLynx™ v4.1). Parallel evaporative light-scattering detection (385-LC, Varian; Agilent Technologies, Wokingham, U.K.) was incorporated into the system via an active splitter (Model EHMA, 10-port valve; Valco Intruments, active split achieved by proprietary Cyclofluidic hardware). Direct injection mass spectrometry was carried out on a ThermoQuest Finnigan LCQduo employing Xcalibur® v2.0 SR2, Tune Plus v2.0 and Qual Broswer v2.0 vendor software (ThermoFisher).

The conditions adopted were:

| | |
|---|---|
| Column | Phenomenex Luna C18(2) 5 μm 150 × 4.6 mm. |
| Eluent | Aqueous phase - Water containing |
| | 0.2% v/v trifluoroacetic acid. |
| | Organic phase - Acetonitrile containing |
| | 0.2% v/v trifluoroacetic acid. |
| Temperature | Ambient |
| Detection | Mass spectrometry - ESI + over m/z range 150 to 850. |
| | UV - Diode array over range 220 to 400 nm. |
| | ELSD - Evaporator at 35° C., nebuliser at 35° C. |
| | and gas flow at 1.8 L/min. |

Equilibration was achieved using a start-up method ahead of the next sample run.

### Example E32—Preparation of Compounds by Flow Chemistry (Alkylation Followed by Suzuki)

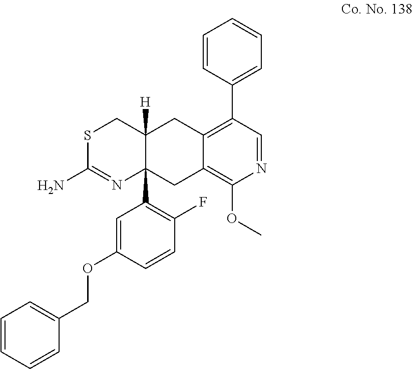 [SMILES: COc1ncc(-c2ccccc2)c2c1C[C@]1(c3cc(OCc4ccccc4)ccc3F)N=C(N)SC[C@@H]1C2]

In one vessel was placed I-47 (15 mg, 0.028 mmol) dissolved in NMP (0.25 mL) and DBU (0.012 mL, 0.084 mmol) in a second vessel was placed e.g. benzyl bromide (6.62 μL, 0.056 mmol) in NMP (0.25 mL). These materials were automatically injected into 250 L injection loops using a Gilson 215 and subsequently mixed in a 2 mL heated coil at 80° C. for 20 min. To the outflow was mixed e.g. phenylboronic acid (5.10 mg, 0.042 mmol) and K₂CO₃ (11.55 mg, 0.084 mmol)) in IPA (0.100 mL), THF (0.100 mL) and water (0.05 mL) loaded into a 250 μL injection loop and Pd(dppf)Cl₂ (1.059 mg, 1.393 mmol) in IPA (0.25 mL) and THF (0.25 mL) loaded to a 250 μL injection loop. The material was heated to 140° C. for 10 min in a 2 mL heated coil. The product was passed to an injection valve and purified as described in example E31, to yield Co. No. 138 (yield 4%).

### Example E33—Preparation of Compound 188

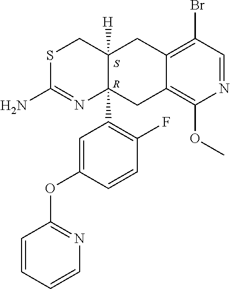

A mixture of I-46 (50 mg, 0.0929 mmol), Cs₂CO₃ (90.768 mg, 0.279 mmol), and 2-fluoropyridine ([372-48-5], 9.787 μL, 0.111 mmol) in DMF (1.192 mL) was heated to 100° C. for 3 h. The solvent was removed by evaporation. A was purified by column chromatography (silica, NH₃ 7M in MeOH/DCM 0/100 to 4/96). The desired fractions were collected and the solvent evaporated in vacuo. The compound was triturated with heptane to yield Co. No. 188 (20.5 mg, 43%) as a yellowish solid.

Tables 1 to 4 below list the compounds of Formula (I) and (II) that were exemplified (*Ex. No.) and prepared by analogy to one of the above Examples (indicated by the Ex. No.). In case no salt form is indicated, the compound was obtained as a free base. ‘Ex. No.’ refers to the Example number according to which protocol the compound was synthesized. ‘Co. No.’ means compound number.

TABLE 1a
Compounds of Formula (I) isolated as a racemic mixture of single cis
diastereomers wherein X = S.

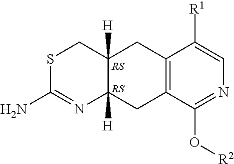

| Co. | | | | |
|---|---|---|---|---|
| No. | Ex | R¹ | R | R² |
| 1 | *E1 | H | 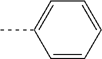 | CH₃ |
| 2 | *E2 | H | 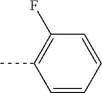 | CH₃ |
| 3 | *E6 | H | 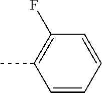 | H |
| 4 | E15 | H | 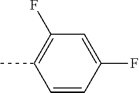 | CH₂CH₃ |
| 5 | E13 | H | 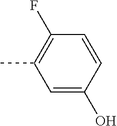 | CH₃ |
| 6 | E1 | H | 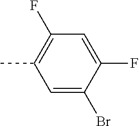 | CH₃ |
| 7 | E1 | H | 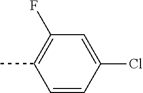 | CH₃ |
| 8 | E1 | H | 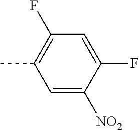 | CH₃ |
| 9 | E16 | H | 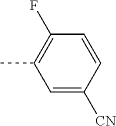 | CH₃ |
| 10 | *E22 | H | 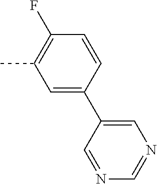 | CH₃ |
| 11 | E22 | H | 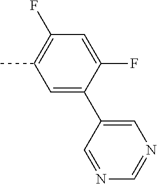 | CH₃ |
| 12 | *E21 | H | 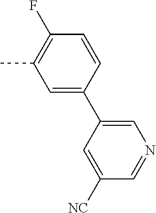 | CH₃ |
| 13 | E16 | H | 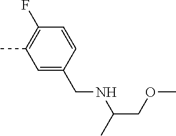 | CH₃ |
| 14 | E14 | H | 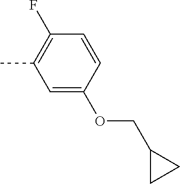 | CH₃ |
| 15 | E1 | H | 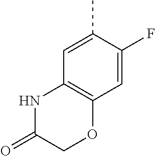 | CH₃ |
| 16 | *E4 | Br | 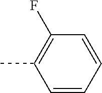 | CH₃ |
| 17 | E1 | Br | 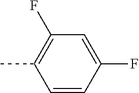 | CH₃ |
| 18 | E16 | Br | 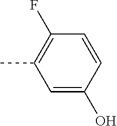 | CH₃ |
| 19 | E16 | Br | 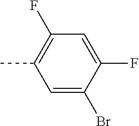 | CH₃ |
| 20 | *E3 | 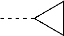 | 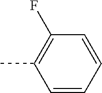 | CH₃ |
| 21 | E3 | 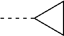 | 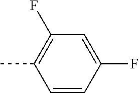 | CH₂CH₃ |
| 22 | *E8 | CN | 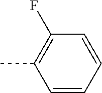 | CH₃ |
| 23 | E8 | CN | 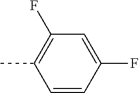 | CH₃ |
| 24 | *E10 | —(C═O)OCH₂CH₃ | 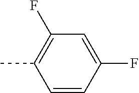 | CH₃ |
| 25 | *E9 | 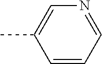 | 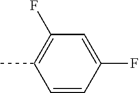 | CH₃ |
| 26 | E1 | 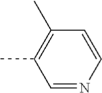 | 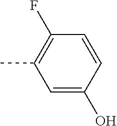 | CH₃ |
| 27 | *E20 | 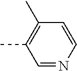 | 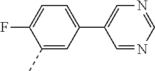 | CH₃ |
| 28 | E1 | 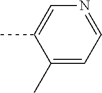 | 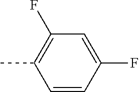 | CH₃ |
| 29 | *E12 | 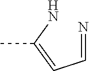 | 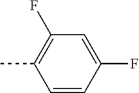 | CH₃ |
| 204 | *E27 | H | 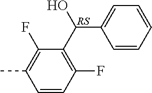 | CH₃ |
| 205 | E27 | Br | 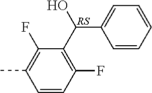 | CH₃ |
| 206 | E27 | H | 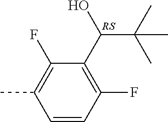 | CH₃ |
| 207 | *E28 | H | 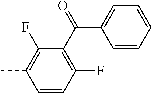 | CH₃ |
| 208 | E28 | H | 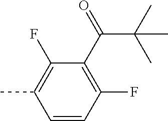 | CH₃ |

TABLE 1b
Compounds of Formula (I) isolated as a racemic mixture of single cis
diastereomers wherein X = O

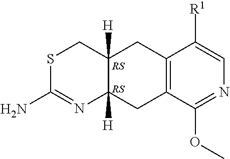

| Co. | | | |
|---|---|---|---|
| No. | Ex. | R¹ | R |
| 30 | *E23 | H | 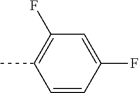 |
| 31 | *E24 | 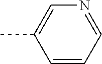 | 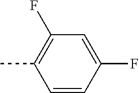 |
| 32 | E24 | 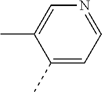 | 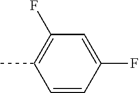 |
| 33 | E24 | 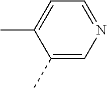 | 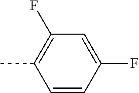 |

TABLE 2
Compounds of Formula (II) isolated as a racemic mixture of single cis
diastereomers wherein X = S.

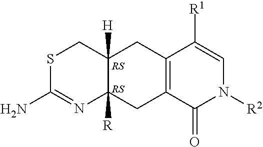

| Co. | | | | |
|---|---|---|---|---|
| No. | Ex | R¹ | R | R² |
| 3 | *E6 | H | 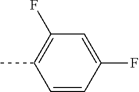 | H |
| 34 | *E5 | H | 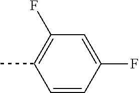 | CH₃ |

TABLE 3a
Compounds of Formula (I) isolated enantiopure of C₄ₐ(R)C₁₀ₐ(S) stereoconfiguration.

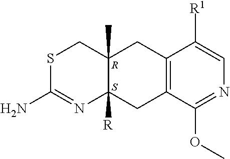

| Co. | | | | |
|---|---|---|---|---|
| No. | Ex. | R¹ | R | SALT |
| 35 | *E7 | H | 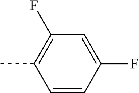 | |
| 36 | *E13 | H | 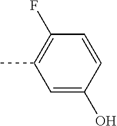 | |
| 37 | *E21 | H | 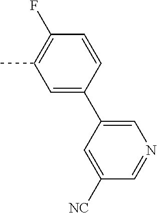 | |
| 38 | *E22 | H | 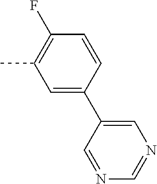 | |
| 39 | E1 | 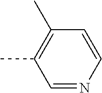 | 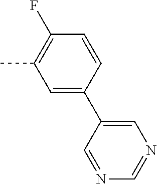 | |
| 40 | E1 | Br | 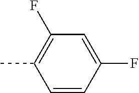 | |
| 41 | E1 | Cl | 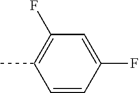 | |
| 42 | *E16 | Br | 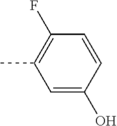 | |
| 43 | E11 | CH₂OH | 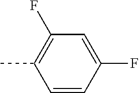 | |
| 44 | E11 | CH₂OCH₃ | 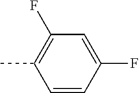 | |
| 45 | E11 | 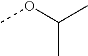 | 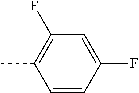 | |
| 46 | E26 | 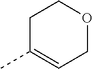 | 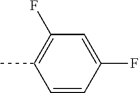 | |
| 47 | *E30 | 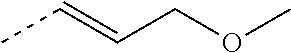 | 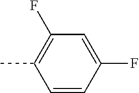 | |
| 48 | E11 | 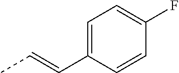 | 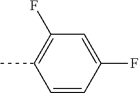 | |
| 49 | E11 | 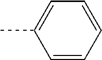 | 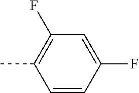 | |
| 50 | E31 | 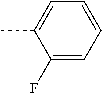 | 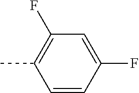 | |
| 51 | E31 | 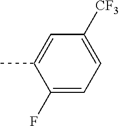 | 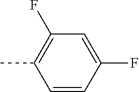 | |
| 52 | E15 | 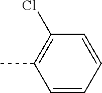 | 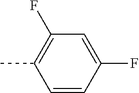 | |
| 53 | E15 | 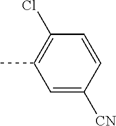 | 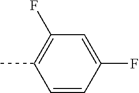 | |
| 54 | E31 | 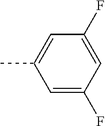 | 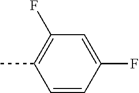 | |
| 55 | E15 | 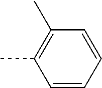 | 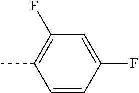 | |
| 56 | E31 | 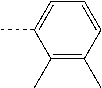 | 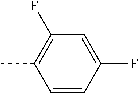 | |
| 57 | E26 | 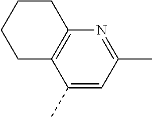 | 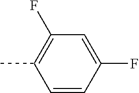 | |
| 58 | E31 | 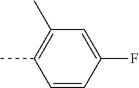 | 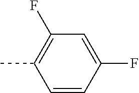 | |
| 59 | E31 | 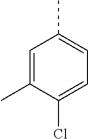 | 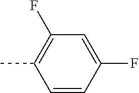 | |
| 60 | E31 | 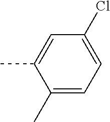 | 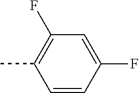 | |
| 61 | E31 | 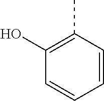 | 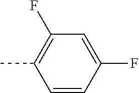 | |
| 62 | E31 | 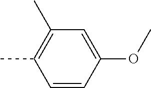 | 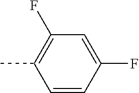 | |
| 63 | E31 | 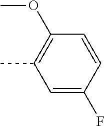 | 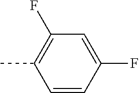 | |
| 64 | E31 | 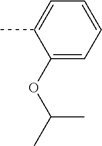 | 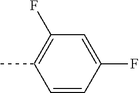 | |
| 65 | E31 | 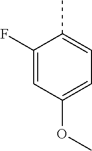 | 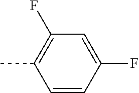 | |
| 66 | E31 | 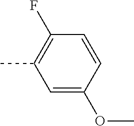 | 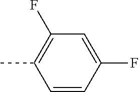 | |
| 67 | E31 | 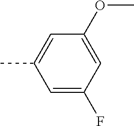 | 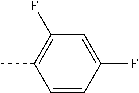 | |
| 68 | E31 | 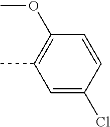 | 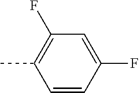 | |
| 69 | E31 | 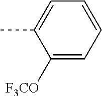 | 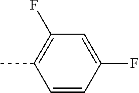 | |
| 70 | E31 | 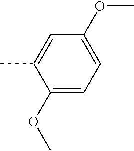 | 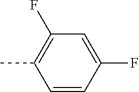 | |
| 71 | E31 | 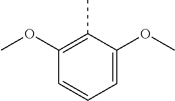 | 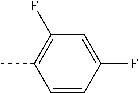 | |
| 72 | E31 | 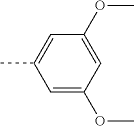 | 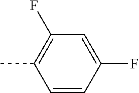 | |
| 73 | E31 | 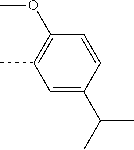 | 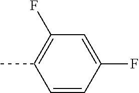 | |
| 74 | E31 | 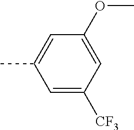 | 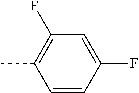 | |
| 75 | E31 | 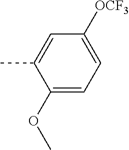 | 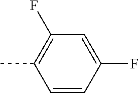 | |
| 76 | E15 | 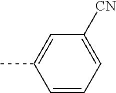 | 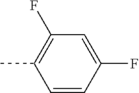 | |
| 77 | E15 | 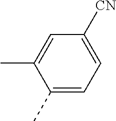 | 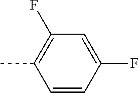 | |
| 78 | E31 | 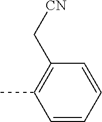 | 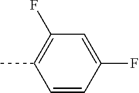 | |
| 79 | E15 | 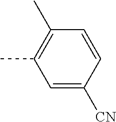 | 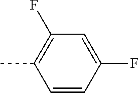 | |
| 80 | E15 | 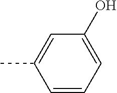 | 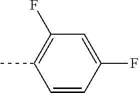 | |
| 81 | E11 | 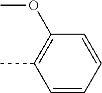 | 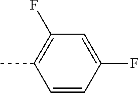 | |
| 82 | E15 | 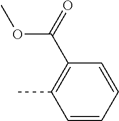 | 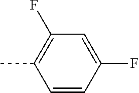 | |
| 83 | E31 | 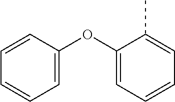 | 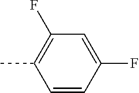 | |
| 84 | E31 | 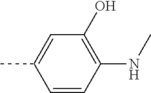 | 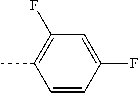 | |
| 85 | E31 | 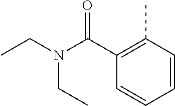 | 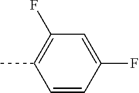 | |
| 86 | E31 | 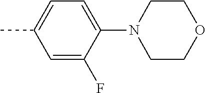 | 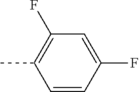 | |
| 87 | *E11 | 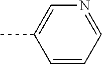 | 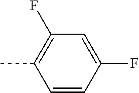 | |
| 88 | E15 | 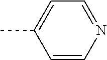 | 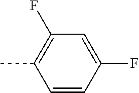 | |
| 89 | E11 | 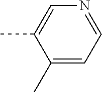 | 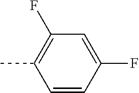 | |
| 90 | E15 | 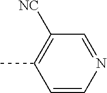 | 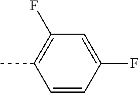 | |
| 91 | E15 | 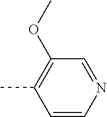 | 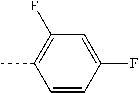 | |
| 92 | E15 | 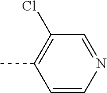 | 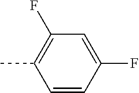 | |
| 93 | E11 | 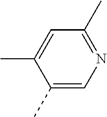 | 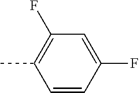 | |
| 94 | E26 | 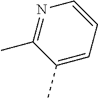 | 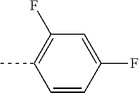 | |
| 95 | E26 | 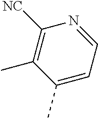 | 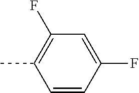 | |
| 96 | E31 | 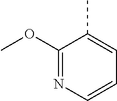 | 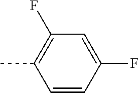 | |
| 97 | E31 | 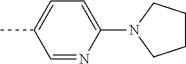 | 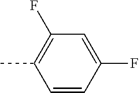 | |
| 98 | E31 | 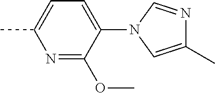 | 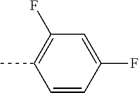 | |
| 99 | E31 | 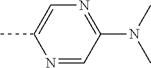 | 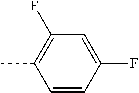 | |
| 100 | E31 | 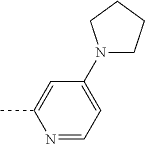 | 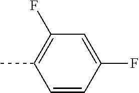 | |
| 101 | E31 | 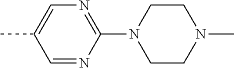 | 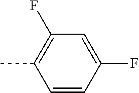 | |
| 102 | E15 | 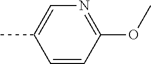 | 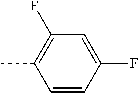 | |
| 103 | *E18 | 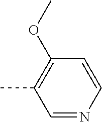 | 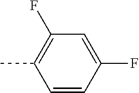 | |
| 104 | E11 | 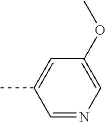 | 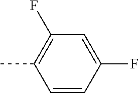 | |
| 105 | E26 | 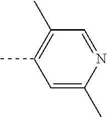 | 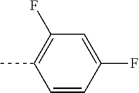 | |
| 106 | *E15 | 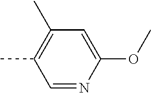 | 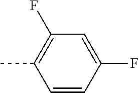 | |
| 107 | E15 | 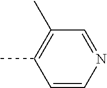 | 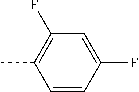 | |
| 108 | *E17 | 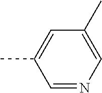 | 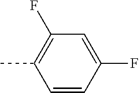 | |
| 109 | E26 | 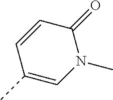 | 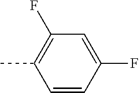 | |
| 110 | E15 | 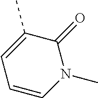 | 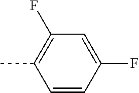 | |
| 111 | E26 | 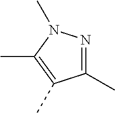 | 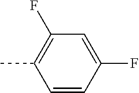 | |
| 112 | E31 | 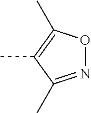 | 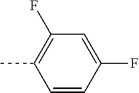 | |
| 113 | E31 | 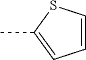 | 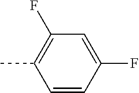 | |
| 114 | E31 | 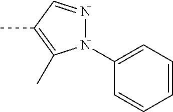 | 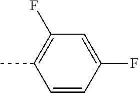 | |
| 115 | E31 | 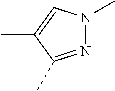 | 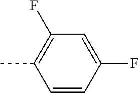 | |
| 116 | E31 | 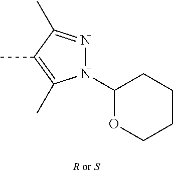 | 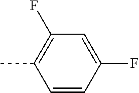 | |
| 117 | E31 | 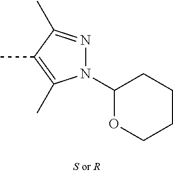 | 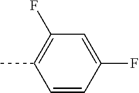 | |
| 118 | E31 | 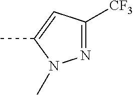 | 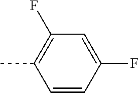 | |
| 119 | E31 | 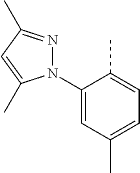 | 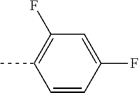 | |
| 120 | E31 | 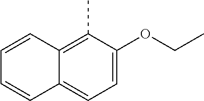 | 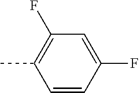 | |
| 121 | E31 | 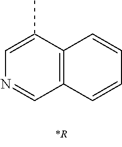 | 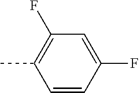 | |
| 122 | E31 | 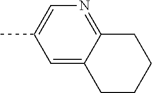 | 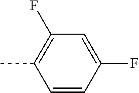 | |
| 123 | E31 | 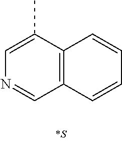 | 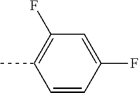 | |
| 124 | E31 | 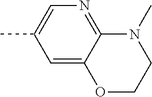 | 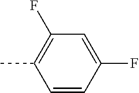 | |
| 125 | E31 | 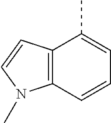 | 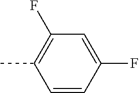 | |
| 126 | E31 | 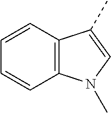 | 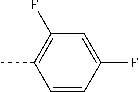 | |
| 127 | E31 | 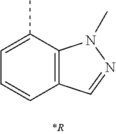 | 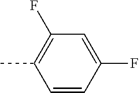 | |
| 128 | E31 | 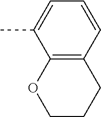 | 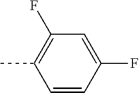 | |
| 129 | E31 | 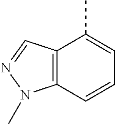 | 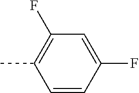 | |
| 130 | E31 | 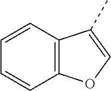 | 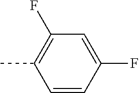 | |
| 131 | E31 | 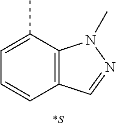 | 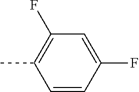 | |
| 132 | E31 | 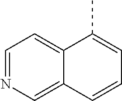 | 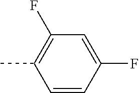 | |
| 133 | E31 | 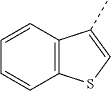 | 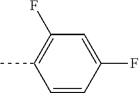 | |
| 134 | E11 | 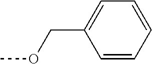 | 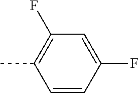 | |
| 135 | E11 | 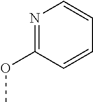 | 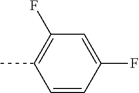 | |
| 136 | *E14 | H | 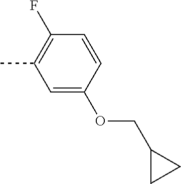 | |
| 137 | E14 | Br | 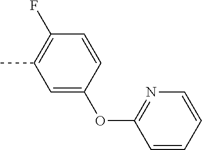 | |
| 138 | *E32 | 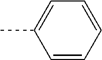 | 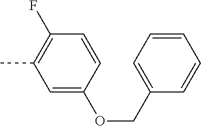 | |
| 139 | E32 | 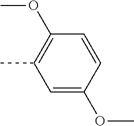 | 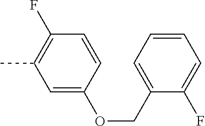 | |
| 140 | E32 | 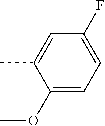 | 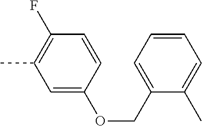 | |
| 141 | E32 | 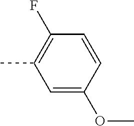 | 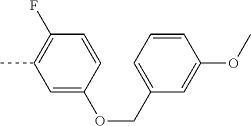 | |
| 142 | E32 | 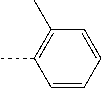 | 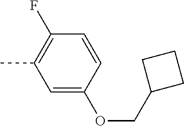 | |
| 143 | E32 | 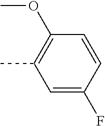 | 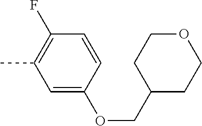 | |
| 144 | E32 | 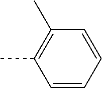 | 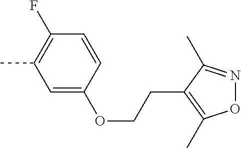 | |
| 145 | E32 | 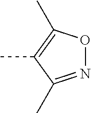 | 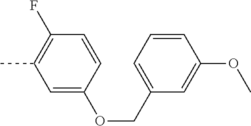 | |
| 146 | E32 | 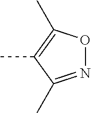 | 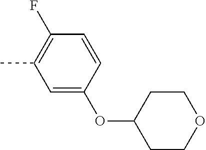 | |
| 147 | E32 | 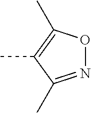 | 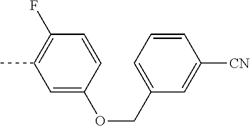 | |
| 148 | E32 | 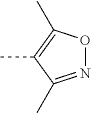 | 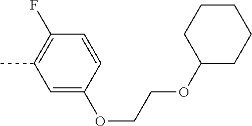 | |
| 149 | E32 | 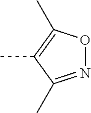 | 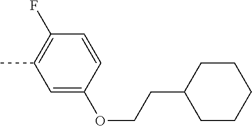 | |
| 150 | E32 | 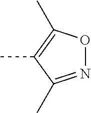 | 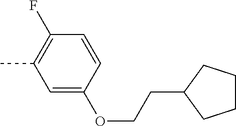 | |
| 151 151a | E32 | 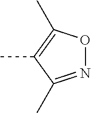 | 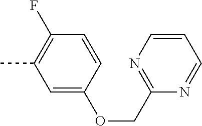 | •HCO₂H |
| 152 | E32 | 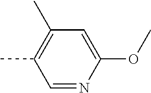 | 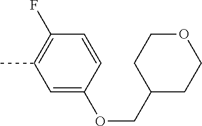 | |
| 153 | E32 | 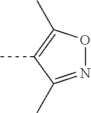 | 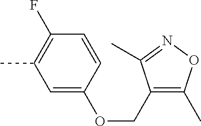 | |
| 154 | E32 | 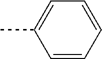 | 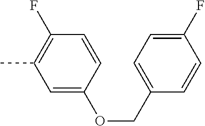 | |
| 155 | E32 | 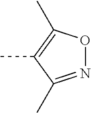 | 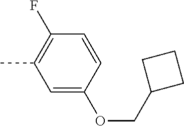 | |
| 156 | E32 | 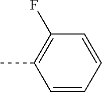 | 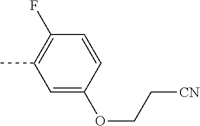 | |
| 157 | E32 | 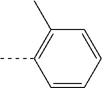 | 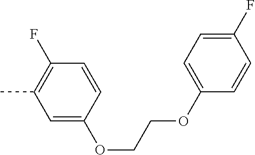 | |
| 158 | E32 | 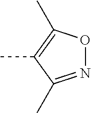 | 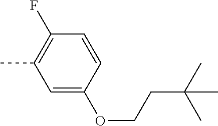 | |
| 159 | E32 | 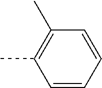 | 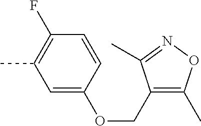 | |
| 160 | E32 | 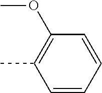 | 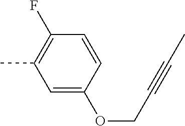 | |
| 161 | E32 | 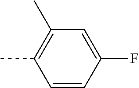 | 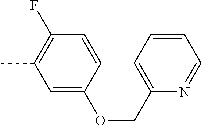 | |
| 162 | E32 | 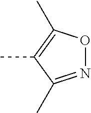 | 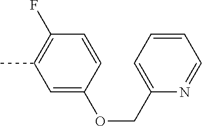 | |
| 163 | E32 | 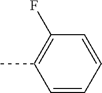 | 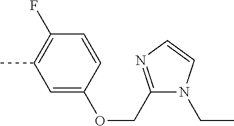 | |
| 164 | E32 | 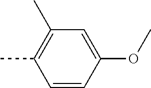 | 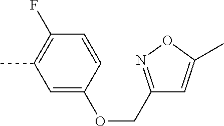 | |
| 165 | E32 | 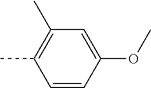 | 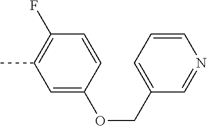 | |
| 166 | E32 | 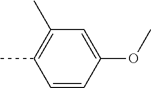 | 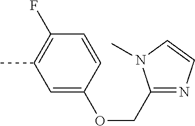 | |
| 167 | E32 | 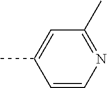 | 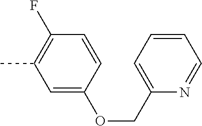 | |
| 168 | E32 | 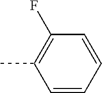 | 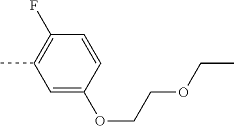 | |
| 169 | E32 | 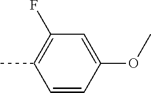 | 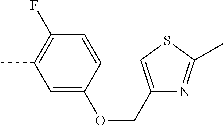 | |
| 170 | E32 | 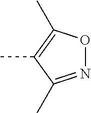 | 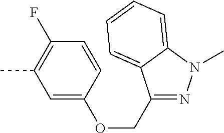 | |
| 171 171a | E32 | 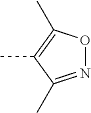 | 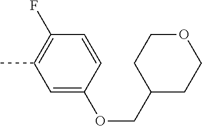 | •HCO₂H |
| 172 | E32 | 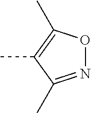 | 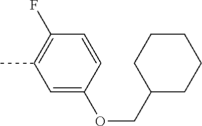 | |
| 173 | E32 | 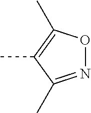 | 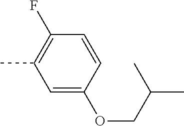 | |
| 174 174a | E32 E32 | 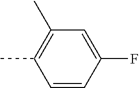 | 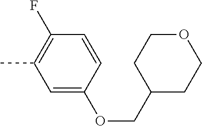 | •HCO₂H |
| 175 | E32 | 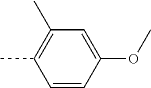 | 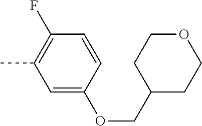 | |
| 176 | E32 | 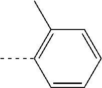 | 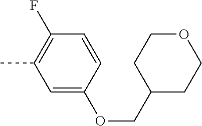 | |
| 177 | E32 | 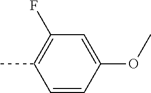 | 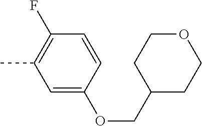 | |
| 178 | E32 | 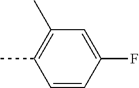 | 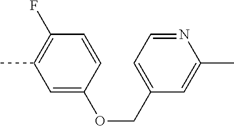 | |
| 179 | E32 | 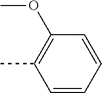 | 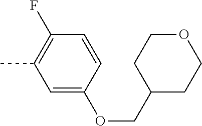 | |
| 180 180a | E32 E32 | 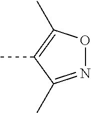 | 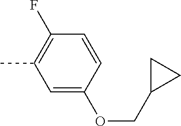 | •HCO₂H |
| 181 181a | E32 E32 | 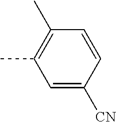 | 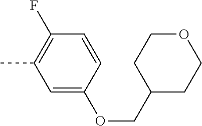 | •HCO₂H |
| 202 | E26* | 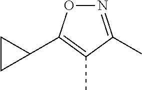 | 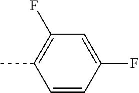 | |
| 203 | E26 | 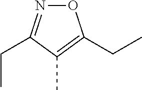 | 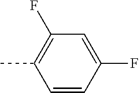 | |
| 209 | E29* | 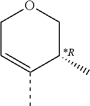 | 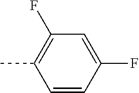 | |
| 210 | E29* | 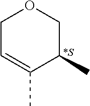 | 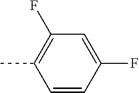 | |
| 211 | E29 | 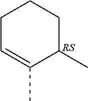 | 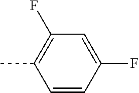 | |
| 212 | E29 | 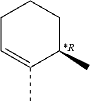 | 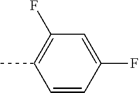 | |
| 213 | E29 | 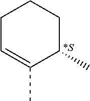 | 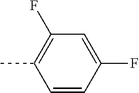 | |

TABLE 3b
Compounds of Formula (I) isolated enantiopure of
C₄ₐ(S)C₁₀ₐ(R) stereoconfiguration.

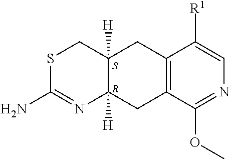

| Co. | | | |
|---|---|---|---|
| No. | Ex. | R¹ | R |
| 182 | *E7 | H | 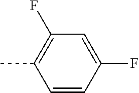 |
| 183 | *E13 | H | 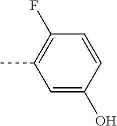 |
| 184 | *E14 | H | 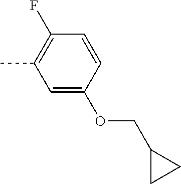 |
| 185 | *E22 | H | 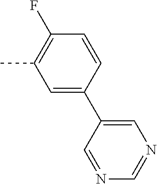 |
| 186 | *E21 | H | 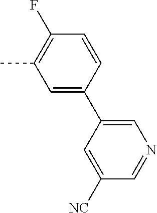 |
| 187 | E1 | Br | 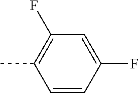 |
| 188 | *E33 | Br | 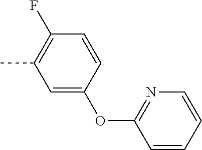 |
| 189 | E4 | Br | 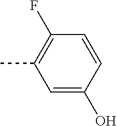 |
| 190 | E15 | 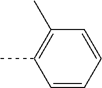 | 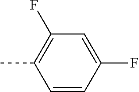 |
| 191 | E15 | 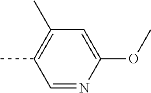 | 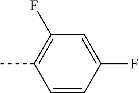 |
| 192 | E15 | 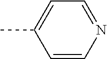 | 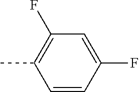 |
| 193 | E1 | 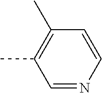 | 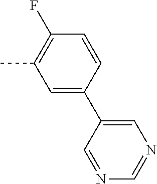 |

TABLE 4
Compounds of Formula (I) isolated enantiopure of unknown C₄ₐ(R)C₁₀ₐ(S) or
C₄ₐ(S)C₁₀ₐ(R) stereoconfiguration.

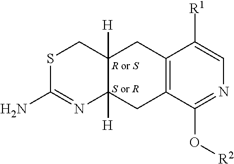

| Co. | | | | | |
|---|---|---|---|---|---|
| No. | Ex. | R¹ | R | R² | Enantiomer |
| 194 | E1 | H | 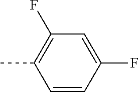 | OCH₂CH₃ | A |
| 195 | E1 | H | 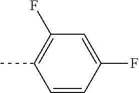 | OCH₂CH₃ | B |
| 196 | E1 | H | 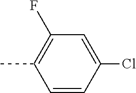 | OCH₂CH₃ | A |
| 197 | E1 | H | 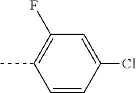 | OCH₂CH₃ | B |
| 198 | E1 | H | 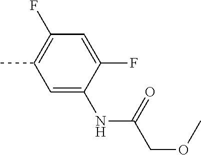 | OCH₃ | A |
| 199 | E1 | H | 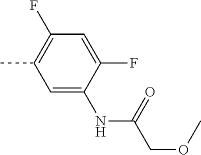 | OCH₃ | B |
| 200 | E26 | 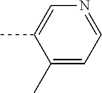 | 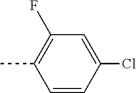 | OCH₃ | A |
| 201 | E26 | 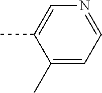 | 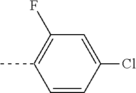 | OCH₃ | B |

### C. Analytical Part

### Melting Points

Values are either peak values or melt ranges, and are obtained with experimental uncertainties that are commonly associated with this analytical method.

### DSC823e

For a number of compounds, melting points were determined with a DSC823e (Mettler-Toledo). Melting points were measured with a temperature gradient of 10° C./minute. Maximum temperature was 300° C.

### LCMS

### LCMS General Procedure 1

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rt) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc. . . . ). For molecules with multiple isotopic patterns (Br, Cl . . . ), the reported value is the one obtained for the lowest isotope mass. All results were obtained with experimental uncertainties that are commonly associated with the method used.

Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica., “Q-Tof” Quadrupole Time-of-flight mass spectrometers, “CLND”, ChemiLuminescent Nitrogen Detector, “ELSD” Evaporative Light Scanning Detector,

TABLE 5a
LCMS Method codes (Flow expressed in mL/min; column temperature (T) in
° C.; Run time in minutes).

| | | | | | | RUN |
|---|---|---|---|---|---|---|
| | | | | | FLOW | TIME |
| METHOD | INSTRUMENT | COLUMN | MOBILE PHASE | GRADIENT | COL T | (MIN) |
| 1 | Waters: | Waters: BEH | A: 10 mM | From | 0.7 | 1.8 |
| | Acquity ® | C18 | CH₃COONH₄ | 95% A to | 70 | |
| | UPLC ®- | (1.7 μm, | in 95% H2O + | 5% A in | | |
| | DAD and SQD | 2.1 * 50 mm) | 5% CH₃CN | 1.3 min, | | |
| | | | B: CH3CN | held for | | |
| | | | | 0.2 min, | | |
| | | | | to 95% A | | |
| | | | | in 0.2 min | | |
| | | | | held | | |
| | | | | for 0.1 min | | |
| 2 | Waters: | Waters: HSS | A: 10 mM | From | 0.7 | 3.5 |
| | Acquity ® | T3 | CH₃COONH₄ | 100% A | 55 | |
| | UPLC ®- | (1.8 μm, | in 95% H2O + | to | | |
| | DAD and SQD | 2.1 * 100 mm) | 5% CH₃CN | 5% A in | | |
| | | | B: CH3CN | 2.10 min, | | |
| | | | | to 0% A | | |
| | | | | in | | |
| | | | | 0.90 min, | | |
| | | | | to 5% A | | |
| | | | | in 0.5 min | | |
| 3 | Waters: | Waters: BEH | A: 10 mM | From | 0.8 | 2 |
| | Acquity ® | C18 | CH₃COONH₄ | 95% A to | 55 | |
| | UPLC ®- | (1.7 μm, | in 95% H2O + | 5% A in | | |
| | DAD and SQD | 2.1 * 50 mm) | 5% CH₃CN | 1.3 min, | | |
| | | | B: CH3CN | held for | | |
| | | | | 0.7 min | | |
| 4 | Waters: | Waters: HSS | A: 10 mM | From | 0.7 | 3.5 |
| | Acquity ® UPLC ®- | T3 | CH₃COONH₄ | 100% A | 55 | |
| | DAD and SQD | (1.8 μm, | in 95% H2O + | to | | |
| | | 2.1 * 100 mm) | 5% CH₃CN | 5% A in | | |
| | | | B: CH3CN | 2.10 min, | | |
| | | | | to 0% A | | |
| | | | | in | | |
| | | | | 0.90 min, | | |
| | | | | to 5% A | | |
| | | | | in 0.5 min | | |

TABLE 5b
Physico-chemical data for some compounds, retention time (Rₜ)
in min, [M + H]⁺ peak (protonated molecule), LCMS method
and mp (melting point in ° C.).

| CO. | MP | R_{T} | [M + | | OTHER | |
|---|---|---|---|---|---|---|
| NO. | (° C.) | (MIN) | H]⁺ | [M − H]⁻ | M | METHOD |
| 30 | | 0.74 | 346.1 | | | 3 |
| 31 | | 0.87 | 423.2 | 421.3 | | 3 |
| 33 | | 0.89 | 437.2 | | | 3 |
| 32 | | 1.55 | 437.3 | 435.1 | | 2 |
| 188 | | 2.2 | 515 | 513 | | 2 |
| 196 | | 1.96 | 378 | 376 | | 4 |
| 197 | | 1.95 | 378 | 376 | | 4 |
| 77 | | 2.2 | 477 | 475 | | 2 |
| 195 | | 1.92 | 376 | 374 | | 4 |
| 194 | | 1.92 | 376 | 374 | | 4 |
| 137 | | 2.21 | 515 | 513 | | 2 |
| 200 | | 1.99 | 469 | 467 | | 4 |
| 201 | | 1.99 | 469 | 467 | | 4 |
| 92 | | 1.02 | 473.1 | 471 | | 3 |
| 28 | | 1.97 | 467 | 465 | | 4 |
| 45 | | 2.18 | 420 | 418 | | 2 |
| 135 | | 1.97 | 455 | 453 | | 4 |
| 94 | 182.26 | 1.83 | 453 | 451 | | 4 |
| 57 | | 2.11 | 507 | 505 | | 4 |
| 52 | | 1.23 | 472.1 | 470 | | 3 |
| 193 | | 1.58 | 513 | 511.1 | | 2 |
| 39 | | 1.58 | 513 | 511 | | 2 |
| 53 | | 2.13 | 497.3 | 495.2 | | 4 |
| 93 | | 1.04 | 467 | 465.1 | | 3 |
| 95 | | 1.96 | 478.2 | 476.1 | | 2 |
| 46 | | 1.85 | 444 | 442 | | 2 |
| 105 | | 1.93 | 467.4 | 465.3 | | 4 |
| 112 | | 1.96 | 457 | 455 | | 2 |
| 111 | | 0.94 | 470.2 | 468.2 | | 3 |
| 19 | | 2.33 | 517.99 | 515.92 | | 2 |
| 6 | | 2.02 | 440.07 | 437.9 | | 2 |
| 8 | | 1.79 | 407.2 | 465 | | 4 |
| | | | | [MCH₃COO⁻] | | |
| 11 | | 0.84 | 440.2 | 438.1 | | 3 |
| 15 | | 1.48 | 415.2 | 413.1 | | 4 |
| 199 | | 1.52 | 449 | 447 | | 4 |
| 198 | | 1.55 | 449 | 447 | | 4 |
| 66 | | 1.33 | 486.3 | 484.4 | | 1 |
| 181a | | 1.3434 | 573.4 | 571.5 | | 1 |
| 174 | | 1.4284 | 567.4 | 564.5 | | 1 |
| 180a | | 1.1967 | 509.3 | 507.5 | | 1 |
| 47 | | 1.2208 | 432.3 | 430.4 | | 1 |
| 171 | | 1.2917 | 553.4 | 551.5 | | 1 |
| 60 | | 1.4534 | 486.3 | 484.5 | | 1 |
| 151a | | 1.1192 | 547 | | | 1 |
| 207 | | 1.99 | 466.2 | 464.2 | | 4 |
| 203 | | 2.13 | 485.3 | 483.2 | | 4 |
| 208 | | 2.09 | 446.3 | 444.1 | | 4 |
| 213 | | 2.45 | | 454.3 | | 4 |
| 212 | | 2.43 | 456.3 | | 464.4 | 4 |
| 210 | | 1.02 | 458.2 | 456.1 | | 3 |
| 209 | | 1 | 458.2 | | 546.1 | 3 |

### LCMS General Procedure 2

HPLC-MS was carried out using an Acquity™ Ultra Performance LC system, comprising a PDA detector, Binary Solvent Manager and SQ detector (Waters UK Ltd., Elstree, UK), tandem linked to a mass spectrometry system (Waters UK Ltd., Manchester, UK) employing vendor software (OpenLynx Browser™ v4.1, SQ Detector v4.1, Instrument Driver V4.1 and MassLynx™ v4.1). Parallel evaporative light-scattering detection (385-LC, Varian; Agilent Technologies, Wokingham, U.K.) was incorporated into the system via an active splitter (Model EHMA, 10-port valve; Valco Intruments, active split achieved by proprietary Cyclofluidic hardware). Direct injection mass spectrometry was carried out on a ThermoQuest Finnigan LCQduo employing Xcalibur® v2.0 SR2, Tune Plus v2.0 and Qual Broswer v2.0 vendor software (ThermoFisher).

### Conditions:

| | |
|---|---|
| Column | Phenomenex Luna C18(2) 5 μm 150 × 4.6 mm. |
| Eluent | Aqueous phase—Water containing 0.2% v/v |
| | trifluoroacetic acid. |
| | Organic phase—Acetonitrile containing 0.2% v/v |
| | trifluoroacetic acid. |
| Temperature | Ambient |
| Detection | Mass spectrometry—ESI + over m/z range 150 to 850. |
| | UV—Diode array over range 220 to 400 nm. |
| | ELSD—Evaporator at 35° C., nebuliser at 35° C. and gas |
| | flow at 1.8 L/min. |

### Gradient Profile:

| Time | Flow | Amount of organic |
|---|---|---|
| (minutes) | (ml min⁻¹) | phase (%) |
| 0 | 1.5 | 10 |
| 0.2 | 1.5 | 10 |
| 9.0 | 1.5 | 99 |
| 11.0 | 1.5 | 99 |
| 11.1 | 1.5 | 10 |
| 12.0 | 1.5 | 10 |
| 12.1 | 0 | 10 |

Equilibration was achieved using a start-up method ahead of the next sample run.

TABLE 5c
Physico-chemical data for some compounds using
LCMS general procedure 2.

| | R_{T} | |
|---|---|---|
| CO. NO. | (MIN) | [M + H]⁺ |
| 47 | 5.85 | 432.2 |
| 48 | 7.415 | 482.1 |
| 49 | 6.89 | 438.1 |
| 50 | 6.79 | 456.1 |
| 51 | 7.52 | 524.1 |
| 54 | 7.27 | 474.1 |
| 55 | 7.14 | 452.2 |
| 56 | 7.44 | 466.2 |
| 58 | 7.2 | 470.1 |
| 59 | 7.93 | 486.1 |
| 60 | 7.69 | 486.1 |
| 61 | 5.9 | 454.1 |
| 62 | 7.03 | 482.2 |
| 63 | 6.77 | 486.1 |
| 64 | 7.14 | 496.2 |
| 65 | 6.8 | 486.1 |
| 66 | 6.86 | 486.1 |
| 67 | 7.18 | 486.1 |
| 68 | 7.27 | 502.1 |
| 69 | 7.19 | 522.1 |
| 70 | 6.61 | 498.2 |
| 71 | 6.148 | 498.2 |
| 72 | 6.94 | 498.2 |
| 73 | 7.81 | 510.2 |
| 74 | 7.75 | 536.1 |
| 75 | 7.69 | 540.1 |
| 78 | 6.68 | 477.2 |
| 81 | 6.59 | 468.2 |
| 83 | 7.57 | 530.2 |
| 84 | 5.99 | 483.2 |
| 85 | 5.93 | 537.2 |
| 86 | 6.85 | 541.2 |
| 96 | 6.14 | 469.1 |
| 97 | 6.68 | 508.2 |
| 98 | 6.17 | 549.2 |
| 99 | 5.87 | 483.2 |
| 100 | 6.504 | 508.2 |
| 101 | 5.47 | 538.2 |
| 106 | 6.5 | 483.2 |
| 112 | 6.02 | 457.1 |
| 112 | 5.79 | 457.1 |
| 113 | 6.87 | 444.1 |
| 114 | 6.63 | 518.2 |
| 115 | 5.36 | 456.2 |
| 116 | 6.21 | 540.2 |
| 117 | 6.22 | 540.2 |
| 118 | 5.95 | 510.1 |
| 119 | 6.68 | 546.2 |
| 120 | 7.2 | 532.2 |
| 121 | 6.12 | 489.2 |
| 122 | 6.54 | 493.2 |
| 123 | 6.21 | 489.2 |
| 124 | 6.18 | 510.2 |
| 125 | 6.88 | 491.2 |
| 126 | 6.93 | 491.2 |
| 127 | 5.24 | 492.2 |
| 128 | 6.94 | 494.2 |
| 129 | 6.33 | 492.2 |
| 130 | 7.19 | 478.1 |
| 131 | 6.79 | 492.2 |
| 132 | 6.27 | 489.2 |
| 133 | 7.52 | 494.1 |
| 138 | 7.68 | 526.2 |
| 139 | 7.38 | 604.2 |
| 140 | 7.75 | 588.2 |
| 141 | 7.66 | 604.2 |
| 142 | 8.28 | 518.2 |
| 143 | 6.96 | 582.2 |
| 144 | 7.2 | 573.2 |
| 145 | 6.95 | 575.2 |
| 146 | 5.99 | 539.2 |
| 147 | 6.58 | 570.2 |
| 148 | 7.44 | 581.3 |
| 149 | 8.61 | 565.3 |
| 150 | 8.2 | 551.2 |
| 151 | 4.67 | 547.2 |
| 152 | 6.61 | 579.2 |
| 153 | 6.15 | 564.2 |
| 154 | 7.66 | 544.2 |
| 155 | 7.3 | 523.2 |
| 156 | 7.32 | 507.2 |
| 157 | 7.97 | 588.2 |
| 158 | 7.85 | 539.2 |
| 159 | 7.11 | 559.2 |
| 160 | 7.32 | 518.2 |
| 161 | 6.79 | 559.2 |
| 162 | 5.17 | 546.2 |
| 163 | 6.2 | 562.2 |
| 164 | 6.73 | 575.2 |
| 165 | 7.23 | 571.2 |
| 166 | 6.09 | 574.2 |
| 167 | 5.51 | 542.2 |
| 168 | 6.76 | 526.2 |
| 169 | 6.81 | 595.2 |
| 170 | 6.64 | 599.2 |
| 171 | 6.04 | 553.2 |
| 172 | 8.19 | 551.2 |
| 173 | 7.14 | 511.2 |
| 174 | 7.37 | 566.2 |
| 174 | 7.33 | 566.2 |
| 175 | 7.12 | 578.2 |
| 176 | 7.24 | 548.2 |
| 177 | 7.05 | 582.2 |
| 178 | 6.98 | 573.2 |
| 179 | 6.77 | 564.2 |
| 180 | 6.63 | 509.2 |
| 181 | 6.81 | 573.2 |

### SFCMS

### General Procedure

The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow cell standing up to 400 bars. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

TABLE 6a
Analytical SFC-MS Methods (Flow expressed in mL/min; column
temperature (T) in ° C.; Run time in minutes, Backpressure (BPR) in bars).

| | | MOBILE | | FLOW | RUN TIME |
|---|---|---|---|---|---|
| METHOD | COLUMN | PHASE | GRADIENT | COL T | BPR |
| 1 | Daicel Chiralpak ® | A: CO2 | 30% B hold 4 min, | 5 | 7 |
| | AD column (5.0 | B: EtOH + 0.2% | to 50% in 1 min, | 40 | 110 |
| | μm, 250 × 4.6 mm) | iPrNH2 | hold 2 min | | |
| 2 | Daicel Chiralpak ® | A: CO2 | 15% B hold 4 min, | 5 | 7 |
| | AS column (5.0 | B: EtOH + 0.2% | to 50% in 1 min, | 40 | 110 |
| | μm, 250 × 4.6 mm) | iPrNH2 | hold 2 min | | |
| 3 | Regis Whelk-O ® 1 | A: CO2 | 35% B hold 4 min, | 5 | 7 |
| | (R,R) column (5.0 | B: EtOH + 0.2% | to 50% in 1 min, | 40 | 110 |
| | μm, 250 × 4.6 mm) | iPrNH2 | hold 2 min | | |
| 4 | Daicel Chiralpak ® | A: CO2 | 20% B hold 4 min, | 5 | 7 |
| | AD column (5.0 | B: EtOH—iPrOH + | to 50% in 1 min, | 40 | 110 |
| | μm, 250 × 4.6 mm) | 0.2% iPrNH2 | hold 2 min | | |
| 5 | Regis Whelk-O ® 1 | A: CO2 | 30% B hold 6 min, | 5 | 7 |
| | (R,R) column (5.0 | B: EtOH—iPrOH + | to 50% in 1 min, | 40 | 110 |
| | μm, 250 × 4.6 mm) | 0.2% iPrNH2 | hold 2.5 min | | |

TABLE 6a
Analytical SFC data—Rₜ means retention time (in minutes),
[M +H]⁺ means the protonated mass of the compound, method refers
to the method used for (SFC)MS analysis of enantiomerically pure
compounds.

| | | | ISOMER | |
|---|---|---|---|---|
| | | | ELUTION | |
| CO. NO. | R_{T} | [M + H]⁺ | ORDER | METHOD |
| 196 | 1.83 | 378 | A | 1 |
| 197 | 2.22 | 378 | B | 1 |
| 195 | 1.81 | 376 | A | 2 |
| 194 | 2.53 | 376 | B | 2 |
| 200 | 2.87 | 469 | A | 3 |
| 201 | 3.58 | 469 | B | 3 |
| 193 | 1.25 | 513 | A | 1 |
| 39 | 1.81 | 513 | B | 1 |
| 105 | 2.39 | 467 | A | 4 |
| 199 | 3.48 | 449 | A | 5 |
| 198 | 3.84 | 449 | B | 5 |

Isomer Elution Order: A means first eluting isomer; B means second eluting isomer.

### NMR

For a number of compounds, ¹H NMR spectra were recorded on a Bruker Avance III with a 300 MHz Ultrashield magnet, on a Bruker DPX-400 spectrometer operating at 400 MHz, on a Bruker Avance I operating at 500 MHz, on a Bruker DPX-360 operating at 360 MHz, or on a Bruker Avance 600 spectrometer operating at 600 MHz, using CHLOROFORM-d (deuterated chloroform, CDCl₃) or DMSO-d₆ (deuterated DMSO, dimethyl-d6 sulfoxide) as solvent. Chemical shifts (δ) are reported in parts per million (ppm) relative to tetramethylsilane (TMS), which was used as internal standard.

TABLE 7
1HNMR results

| CO. NO. | ¹HNMR RESULT |
|---|---|
| 1 | ¹H NMR (400 MHz, CHLOROFORM-d) δ ppm 2.65-2.80 (m, 3 H) 2.98- |
| | 3.14 (m, 4 H) 3.90 (s, 3 H) 6.64 (d, J = 5.29 Hz, 1 H) 7.22-7.26 (m, 1 H) 7.30- |
| | 7.39 (m, 4 H) 7.90 (d, J = 5.27 Hz, 1 H) |
| 2 | ¹H NMR (360 MHz, DMSO-d₆) δ ppm 2.65-2.93 (m, 5 H) 2.95-3.07 (m, 2 |
| | H) 3.80 (s, 3 H) 6.16 (s, 2 H) 6.76 (d, J = 5.27 Hz, 1 H) 7.14-7.25 (m, 2 H) |
| | 7.27-7.41 (m, 2 H) 7.86 (d, J = 5.22 Hz, 1 H) |
| 20 | ¹H NMR (360 MHz, CHLOROFORM-d) δ ppm 0.46-0.55 (m, 1 H) 0.64- |
| | 0.74 (m, 1 H) 0.83-0.96 (m, 2 H) 1.64-1.73 (m, 1 H) 2.85-2.97 (m, 3 H) |
| | 2.97-3.13 (m, 2 H) 3.20-3.31 (m, 2 H) 3.87 (s, 3 H) 4.48 (br. s, 2 H) 7.06 |
| | (dd, J = 12.74, 8.05 Hz, 1 H) 7.10-7.17 (m, 1 H) 7.24-7.30 (m, 1 H) 7.35- |
| | 7.43 (m, 1 H) 7.71 (s, 1 H) |
| 16 | ¹H NMR (360 MHz, CHLOROFORM-d) δ ppm 2.71-2.86 (m, 2 H) 2.89 (dd, |
| | J = 11.93, 2.60 Hz, 1 H) 3.01-3.10 (m, 2 H) 3.19-3.31 (m, 2 H) 3.88 (s, 3 H) |
| | 4.52 (br. s, 2 H) 7.01-7.10 (m, 1 H) 7.14 (td, J = 7.60, 1.35 Hz, 1 H) 7.27- |
| | 7.33 (m, 1 H) 7.37 (td, J = 8.12, 1.85 Hz, 1 H) 8.08 (s, 1 H) |
| 34 | ¹H NMR (400 MHz, CHLOROFORM-d) δ ppm 2.46 (dd, J = 17.96, 5.45 Hz, 1 |
| | H) 2.79 (dd, J = 11.91, 3.43 Hz, 1 H) 2.88 (dd, J = 17.95, 10.49 Hz, 1 H) 2.97- |
| | 3.04 (m, 2 H) 3.11-3.17 (m, 1 H) 3.17-3.23 (m, 1 H) 3.48 (s, 3 H) 5.90 (d, |
| | J = 7.27 Hz, 1 H) 7.01 (ddd, J = 12.92, 8.07, 1.21 Hz, 1 H) 7.03 (d, J = 7.05 Hz, 1 |
| | H) 7.09 (td, J = 7.66, 1.25 Hz, 1 H) 7.20-7.26 (m, 1 H) 7.34 (td, J = 8.17, 1.82 |
| | Hz, 1 H) |
| 3 | ¹H NMR (400 MHz, DMSO-d₆) δ ppm 2.51-2.58 (m, 1 H) 2.64-2.83 (m, 4 |
| | H) 2.83-2.89 (m, 1 H) 2.90-2.99 (m, 1 H) 5.97 (d, J = 6.75 Hz, 1 H) 6.35 (br. |
| | s, 2 H) 7.11 (d, J = 6.72 Hz, 1 H) 7.15-7.24 (m, 2 H) 7.27-7.39 (m, 2 H) |
| | 11.24 (br. s, 1 H) |
| 35 | ¹H NMR (360 MHz, DMSO-d₆) δ ppm 2.66-3.03 (m, 7 H) 3.80 (s, 3 H) 6.20 |
| | (br. s., 2 H) 6.76 (d, J = 5.26 Hz, 1 H) 7.11 (td, J = 8.54, 2.62 Hz, 1 H) 7.25 |
| | (ddd, J = 12.41, 9.32, 2.63 Hz, 1 H) 7.33 (td, J = 9.15, 6.89 Hz, 1 H) 7.86 (d, |
| | J = 5.25 Hz, 1 H) |
| 17 | ¹H NMR (360 MHz, DMSO-d₆) δ ppm 2.58-2.71 (m, 1 H) 2.73-2.86 (m, 3 |
| | H) 2.94-3.06 (m, 3 H) 3.81 (s, 3 H) 6.26 (s, 2 H) 7.12 (td, J = 8.49, 2.66 Hz, 1 |
| | H) 7.21-7.39 (m, 2 H) 8.12 (s, 1 H) |
| 182 | ¹H NMR (360 MHz, DMSO-d₆) δ ppm 2.66-3.04 (m, 7 H) 3.80 (s, 3 H) 6.20 |
| | (br. s., 2 H) 6.76 (d, J = 5.45 Hz, 1 H) 7.11 (td, J = 8.56, 2.63 Hz, 1 H) 7.25 |
| | (ddd, J = 12.42, 9.30, 2.63 Hz, 1 H) 7.33 (td, J = 8.94, 7.26 Hz, 1 H) 7.86 (d, |
| | J = 5.25 Hz, 1 H) |
| 22 | ¹H NMR (360 MHz, CHLOROFORM-d) δ ppm 2.82-2.94 (m, 2 H) 3.00- |
| | 3.13 (m, 3 H) 3.19-3.29 (m, 2 H) 3.95 (s, 3 H) 4.28 (br. s, 2 H) 7.07 (dd, |
| | J = 12.75, 8.06 Hz, 1 H) 7.12-7.20 (m, 1 H) 7.27-7.39 (m, 2 H) 8.30 (s, 1 H) |
| 23 | ¹H NMR (360 MHz, DMSO-d₆) δ ppm 2.73-2.88 (m, 3 H) 2.92-3.09 (m, 4 |
| | H) 3.90 (s, 3 H) 6.29 (s, 2 H) 7.12 (td, J = 8.50, 2.67 Hz, 1 H) 7.20-7.39 (m, 2 |
| | H) 8.49 (s, 1 H) |
| 25 | ¹H NMR (360 MHz, DMSO-d₆) δ ppm 2.54-3.11 (m, 7 H) 3.87 (s, 3 H) 6.23 |
| | (s, 2 H) 7.08-7.15 (m, 1 H) 7.21-7.30 (m, 1 H) 7.30-7.40 (m, 1 H) 7.52 |
| | (dd, J = 7.86, 4.76 Hz, 1 H) 7.73-7.80 (m, 1 H) 7.83 (s, 1 H) 8.53 (d, J = 2.26 |
| | Hz, 1 H) 8.61 (dd, J = 4.81, 1.64 Hz, 1 H) |
| 24 | ¹H NMR (360 MHz, DMSO-d₆) δ ppm 1.31 (t, J = 7.08 Hz, 3 H) 2.74-3.25 |
| | (m, 7 H) 3.89 (s, 3 H) 4.23-4.32 (m, 2 H) 6.26 (br. s, 2 H) 7.12 (t, J = 8.32 Hz, |
| | 1 H) 7.19-7.40 (m, 2 H) 8.53 (s, 1 H) |
| 87 | ¹H NMR (360 MHz, DMSO-d₆) δ ppm 2.59-3.13 (m, 7 H) 3.87 (s, 3 H) 6.23 |
| | (s, 2 H) 7.05-7.18 (m, 1 H) 7.20-7.30 (m, 1 H) 7.30-7.39 (m, 1 H) 7.51 |
| | (dd, J = 7.88, 4.84 Hz, 1 H) 7.72-7.79 (m, 1 H) 7.83 (s, 1 H) 8.53 (d, J = 2.21 |
| | Hz, 1 H) 8.61 (dd, J = 4.80, 1.64 Hz, 1 H) |
| 89 | ¹H NMR (400 MHz, DMSO-d₆) δ ppm 2.06-2.13 (m, 3 H) 2.29 (m, J = 17.46, |
| | 8.18 Hz, 1 H) 2.53-2.61 (m, 1 H) 2.74-2.88 (m, 2 H) 2.94-3.04 (m, 2 H) |
| | 3.08-3.28 (m, 1 H) 3.95 (s, 3 H) 5.83 (br. s, 2 H) 6.97-7.08 (m, 2 H) 7.31 (d, |
| | J = 4.87 Hz, 1 H) 7.40 (q, J = 8.33 Hz, 1 H) 7.72 (s, 1 H) 8.27 (br. s., 1 H) 8.46 |
| | (d, J = 4.93 Hz, 1 H) |
| 49 | ¹H NMR (360 MHz, DMSO-d₆) δ ppm 2.52-3.12 (m, 7 H) 3.85 (s, 3 H) 6.24 |
| | (br. s, 2 H) 7.12 (td, J = 8.50, 2.64 Hz, 1 H) 7.20-7.43 (m, 5 H) 7.44-7.52 (m, |
| | 2 H) 7.76 (s, 1 H) |
| 184 | ¹H NMR (360 MHz, DMSO-d₆) δ ppm 0.27-0.35 (m, 2 H) 0.54-0.60 (m, 2 |
| | H) 1.16-1.27 (m, 1 H) 2.69-2.89 (m, 5 H) 2.93-3.02 (m, 2 H) 3.75 (d, |
| | J = 7.01 Hz, 2 H) 3.80 (s, 3 H) 6.17 (s, 2 H) 6.75 (d, J = 5.27 Hz, 1 H) 6.80- |
| | 6.89 (m, 2 H) 7.04-7.17 (m, 1 H) 7.85 (d, J = 5.23 Hz, 1 H) |
| 136 | ¹H NMR (400 MHz, DMSO-d₆) δ ppm 0.28-0.33 (m, 2 H) 0.54-0.59 (m, 2 |
| | H) 1.17-1.26 (m, 1 H) 2.67-2.89 (m, 5 H) 2.93-3.01 (m, 2 H) 3.75 (d, |
| | J = 6.86 Hz, 2 H) 3.80 (s, 3 H) 6.14 (s, 2 H) 6.75 (d, J = 5.25 Hz, 1 H) 6.82- |
| | 6.87 (m, 2 H) 7.10 (dd, J = 12.31, 9.08 Hz, 1 H) 7.85 (d, J = 5.25 Hz, 1 H) |
| 42 | ¹H NMR (360 MHz, DMSO-d₆) δ ppm 2.61-2.85 (m, 4 H) 2.91-3.06 (m, 3 |
| | H) 3.81 (s, 3 H) 6.20 (br. s., 2 H) 6.66 (dt, J = 8.60, 3.56 Hz, 1 H) 6.74 (dd, |
| | J = 6.77, 3.11 Hz, 1 H) 6.99 (dd, J = 12.42, 8.77 Hz, 1 H) 8.11 (s, 1 H) 9.39 (br. |
| | s, 1 H) |
| 10 | ¹H NMR (400 MHz, DMSO-d₆) δ ppm 2.71-2.95 (m, 5 H) 2.99-3.09 (m, 2 |
| | H) 3.81 (s, 3 H) 6.21 (s, 2 H) 6.77 (d, J = 5.30 Hz, 1 H) 7.42 (dd, J = 12.17, 8.44 |
| | Hz, 1 H) 7.66 (dd, J = 7.70, 2.56 Hz, 1 H) 7.82 (ddd, J = 8.42, 4.42, 2.49 Hz, 1 |
| | H) 7.87 (d, J = 5.19 Hz, 1 H) 9.02 (s, 2 H) 9.20 (s, 1 H) |
| 5 | ¹H NMR (360 MHz, DMSO-d₆) δ ppm 2.61-3.07 (m, 7 H) 3.80 (s, 3 H) 6.15 |
| | (br. s, 2 H) 6.64 (dt, J = 8.67, 3.42 Hz, 1 H) 6.70-6.80 (m, 2 H) 6.98 (dd, |
| | J = 12.20, 8.68 Hz, 1 H) 7.85 (d, J = 5.26 Hz, 1 H) 9.37 (br. s, 1 H) |
| 76 | ¹H NMR (360 MHz, CHLOROFORM-d) δ ppm 2.38 (dd, J = 17.66, 5.33 Hz, 1 |
| | H) 2.68-2.87 (m, 2 H) 2.94-3.02 (m, 1 H) 3.04-3.16 (m, 2 H) 3.21-3.32 |
| | (m, 1 H) 3.95 (s, 3 H) 4.55 (br. s, 2 H) 6.78-6.92 (m, 2 H) 7.37 (td, J = 9.10, |
| | 6.57 Hz, 1 H) 7.51-7.62 (m, 3 H) 7.66-7.70 (m, 1 H) 7.81 (s, 1 H) |
| 18 | ¹H NMR (400 MHz, DMSO-d₆) δ ppm 2.60-2.72 (m, 1 H) 2.73-2.89 (m, 3 |
| | H) 2.92-3.06 (m, 3 H) 3.82 (s, 3 H) 6.16 (br. s, 2 H) 6.60-6.81 (m, 2 H) 6.98 |
| | (dd, J = 12.20, 8.65 Hz, 1 H) 8.11 (s, 1 H) 9.37 (br. s, 1 H) |
| 102 | ¹H NMR (360 MHz, CHLOROFORM-d) δ ppm 2.38 (dd, J = 17.69, 5.34 Hz, 1 |
| | H) 2.68 (dd, J = 11.91, 2.62 Hz, 1 H) 2.78 (dd, J = 17.69, 11.07 Hz, 1 H) 2.87- |
| | 2.96 (m, 1 H) 2.97-3.08 (m, 2 H) 3.17-3.24 (m, 1 H) 3.88 (s, 3 H) 3.94 (s, 3 |
| | H) 4.46 (br. s, 2 H) 6.71-6.85 (m, 3 H) 7.31 (td, J = 9.10, 6.60 Hz, 1 H) 7.47 |
| | (dd, J = 8.48, 2.47 Hz, 1 H) 7.77 (s, 1 H) 8.06 (d, J = 2.45 Hz, 1 H) |
| 189 | ¹H NMR (360 MHz, DMSO-d₆) δ ppm 2.61-2.85 (m, 4 H) 2.92-3.06 (m, 3 |
| | H) 3.81 (s, 3 H) 6.19 (br. s., 2 H) 6.66 (dt, J = 8.77, 3.47 Hz, 1 H) 6.74 (dd, |
| | J = 6.94, 2.94 Hz, 1 H) 6.99 (dd, J = 12.41, 8.77 Hz, 1 H) 8.11 (s, 1 H) 9.39 (br. |
| | s, 1 H) |
| 9 | ¹H NMR (360 MHz, DMSO-d₆) δ ppm 2.71-3.06 (m, 7 H) 3.80 (s, 3 H) 6.33 |
| | (br. s, 2 H) 6.77 (d, J = 5.27 Hz, 1 H) 7.50 (dd, J = 12.07, 8.46 Hz, 1 H) 7.63 (dd, |
| | J = 7.34, 2.24 Hz, 1 H) 7.87 (d, J = 5.23 Hz, 1 H) 7.90-7.98 (m, 1 H) |
| 7 | ¹H NMR (360 MHz, CHLOROFORM-d) δ ppm 2.65 (dd, J = 17.54, 5.68 Hz, 1 |
| | H) 2.84 (dd, J = 12.00, 2.87 Hz, 1 H) 2.93-3.05 (m, 3 H) 3.12-3.23 (m, 2 H) |
| | 3.91 (s, 3 H) 4.27 (br. s, 2 H) 6.64 (d, J = 5.29 Hz, 1 H) 7.07-7.14 (m, 2 H) |
| | 7.27-7.33 (m, 1 H) 7.90 (d, J = 5.29 Hz, 1 H) |
| 41 | ¹H NMR (360 MHz, DMSO-d₆) δ ppm 2.63-2.89 (m, 4 H) 2.94-3.07 (m, 3 |
| | H) 3.81 (s, 3 H) 6.29 (br. s, 2 H) 7.12 (td, J = 8.50, 2.68 Hz, 1 H) 7.25 (ddd, |
| | J = 12.41, 9.30, 2.67 Hz, 1 H) 7.29-7.38 (m 1 H) 8.01 (s 1 H) |
| 26 | ¹H NMR (400 MHz, CHLOROFORM-d) δ ppm 2.05-2.76 (m, 6 H) 3.00- |
| | 3.32 (m, 4 H) 3.99 (s, 3 H) 6.68 (m, J = 8.50 Hz, 1 H) 6.87-6.97 (m, 1 H) 6.98- |
| | 7.06 (m, 1 H) 7.22-7.28 (m, 1 H) 7.78 (s, 1 H) 8.39 (s, 1 H) 8.48-8.57 (m, 1 H) |
| 27 | ¹H NMR (400 MHz, CHLOROFORM-d) δ ppm 2.04-2.76 (m, 6 H) 3.00 |
| | (ddd, J = 12.02, 4.77, 2.92 Hz, 1 H) 3.11-3.26 (m, 2 H) 3.31-3.40 (m, 1 H) |
| | 3.97 (s, 3 H) 7.20-7.27 (m, 2 H) 7.47-7.54 (m, 1 H) 7.61 (td, J = 8.18, 2.45 |
| | Hz, 1 H) 7.77 (s, 1 H) 8.30-8.42 (m, 1 H) 8.46-8.56 (m, 1 H) 8.92 (s, 2 H) |
| | 9.20 (s, 1 H) |
| 12 | ¹H NMR (400 MHz, DMSO-d₆) δ ppm 2.71-3.11 (m, 7 H) 3.81 (s, 3 H) 6.20 |
| | (br. s., 2 H) 6.77 (d, J = 5.14 Hz, 1 H) 7.42 (dd, J = 12.29, 8.29 Hz, 1 H) 7.67 |
| | (dd, J = 7.71, 2.55 Hz, 1 H) 7.78-7.84 (m, 1 H) 7.87 (d, J = 5.09 Hz, 1 H) 8.51 |
| | (t, J = 2.15 Hz, 1 H) 9.01-9.04 (m, 2 H) |
| 37 | ¹H NMR (360 MHz, DMSO-d₆) δ ppm 2.71-2.95 (m, 5 H) 2.99-3.10 (m, 2 |
| | H) 3.81 (s, 3 H) 6.23 (br. s., 2 H) 6.78 (d, J = 5.30 Hz, 1 H) 7.42 (dd, J = 12.20, |
| | 8.41 Hz, 1 H) 7.67 (dd, J = 7.61, 2.55 Hz, 1 H) 7.81 (ddd, J = 8.11, 4.81, 2.57 |
| | Hz, 1 H) 7.87 (d, J = 5.21 Hz, 1 H) 8.51 (t, J = 2.08 Hz, 1 H) 9.01-9.05 (m, 2 H) |
| 43 | ¹H NMR (400 MHz, CHLOROFORM-d) δ ppm 2.80-2.90 (m, 2 H) 2.94- |
| | 3.06 (m, 3 H) 3.11-3.24 (m, 2 H) 3.91 (s, 3 H) 4.45-4.63 (m, 2 H) 6.79- |
| | 6.89 (m, 2 H) 7.32 (td, J = 9.06, 6.64 Hz, 1 H) 7.61 (s, 1 H) |
| 80 | ¹H NMR (360 MHz, CHLOROFORM-d) δ ppm 2.39 (dd, J = 17.84, 5.79 Hz, 1 |
| | H) 2.73-2.84 (m, 2 H) 3.03-3.18 (m, 3 H) 3.35 (d, J = 17.40 Hz, 1 H) 3.60 (s, |
| | 3 H) 5.91 (t, J = 2.20 Hz, 1 H) 6.54 (dt, J = 7.46, 1.21 Hz, 1 H) 6.75-6.87 (m, 2 |
| | H) 6.92 (td, J = 8.28, 2.67 Hz, 1 H) 7.20 (t, J = 7.83 Hz, 1 H) 7.37 (td, J = 9.08, |
| | 6.47 Hz, 1 H) 7.66 (s, 1 H) |
| 185 | ¹H NMR (360 MHz, DMSO-d₆) δ ppm 2.72-2.95 (m, 5 H) 2.99-3.10 (m, 2 |
| | H) 3.81 (s, 3 H) 6.24 (br. s, 2 H) 6.78 (d, J = 5.32 Hz, 1 H) 7.43 (dd, J = 12.21, |
| | 8.46 Hz, 1 H) 7.65 (dd, J = 7.68, 2.63 Hz, 1 H) 7.82 (ddd, J = 8.43, 4.43, 2.51 |
| | Hz, 1 H) 7.87 (d, J = 5.24 Hz, 1 H) 9.02 (s, 2 H) 9.21 (s, 1 H) |
| 38 | ¹H NMR (360 MHz, DMSO-d₆) δ ppm 2.73-2.94 (m, 5 H) 2.99-3.11 (m, 2 |
| | H) 3.81 (s, 3 H) 6.24 (br. s, 2 H) 6.78 (d, J = 5.26 Hz, 1 H) 7.43 (dd, J = 12.30, |
| | 8.42 Hz, 1 H) 7.65 (dd, J = 7.70, 2.66 Hz, 1 H) 7.82 (ddd, J = 8.43, 4.43, 2.51 |
| | Hz, 1 H) 7.87 (d, J = 5.31 Hz, 1 H) 9.02 (s, 2 H) 9.21 (s, 1 H) |
| 186 | ¹H NMR (360 MHz, DMSO-d₆) δ ppm 2.73-2.94 (m, 5 H) 3.01 (m, J = 17.10 |
| | Hz, 2 H) 3.81 (s, 3 H) 6.23 (br. s, 2 H) 6.78 (d, J = 5.38 Hz, 1 H) 7.42 (dd, |
| | J = 12.16, 8.41 Hz, 1 H) 7.67 (dd, J = 7.68, 2.50 Hz, 1 H) 7.82 (ddd, J = 8.47, |
| | 4.42, 2.52 Hz, 1 H) 7.87 (d, J = 5.24 Hz, 1 H) 8.52 (t, J = 2.11 Hz, 1 H) 9.02- |
| | 9.05 (m, 2 H) |
| 110 | ¹H NMR (360 MHz, CHLOROFORM-d) δ ppm 2.58-2.86 (m, 3 H) 2.97 (dd, |
| | J = 11.96, 4.58 Hz, 1 H) 3.02-3.11 (m, 1 H) 3.15-3.31 (m, 2 H) 3.63 (s, 3 H) |
| | 3.92 (s, 3 H) 4.11 (br. s, 2 H) 6.26 (t, J = 6.77 Hz, 1 H) 6.78-6.88 (m, 2 H) |
| | 7.28-7.41 (m, 3 H) 7.76 (s, 1 H) |
| 44 | ¹H NMR (360 MHz, CHLOROFORM-d) δ ppm 2.81-3.06 (m, 5 H) 3.11- |
| | 3.27 (m, 2 H) 3.35 (s, 3 H) 3.90 (s, 3 H) 4.31 (d, J = 11.53 Hz, 1 H) 4.46 (d, |
| | J = 11.52 Hz, 1 H) 6.79-6.90 (m, 2 H) 7.28-7.39 (m, 1 H) 7.87 (s, 1 H) |
| 79 | ¹H NMR (360 MHz, CHLOROFORM-d) δ ppm 1.98-2.29 (m, 4 H) 2.38- |
| | 2.62 (m, 1 H) 2.69 (m, J = 11.90 Hz, 1 H) 2.91-3.00 (m, 1 H) 3.01-3.18 (m, 2 |
| | H) 3.23-3.31 (m, 1 H) 3.96 (s, 3 H) 4.52 (br. s, 2 H) 6.79-6.90 (m, 2 H) 7.29- |
| | 7.38 (m, 1 H) 7.38-7.46 (m, 2 H) 7.60 (dt, J = 7.89, 2.26 Hz, 1 H) 7.70 (d, |
| | J = 2.36 Hz, 1 H) |
| 13 | ¹H NMR (600 MHz, DMSO-d₆) δ ppm 0.95 (t, J = 6.71 Hz, 3 H) 2.66-3.05 |
| | (m, 8 H) 3.13-3.26 (m, 5 H) 3.63 (dd, J = 13.94, 2.35 Hz, 1 H) 3.72 (dd, |
| | J = 13.79, 2.79 Hz, 1 H) 3.80 (s, 3 H) 6.10 (br. s, 2 H) 6.75 (d, J = 5.28 Hz, 1 H) |
| | 7.12 (dd, J = 12.47, 8.10 Hz, 1 H) 7.23-7.32 (m, 2 H) 7.85 (d, J = 5.25 Hz, 1 H) |
| 109 | ¹H NMR (360 MHz, CHLOROFORM-d) δ ppm 2.47 (dd, J = 17.70, 5.48 Hz, 1 |
| | H) 2.70-3.30 (m, 6 H) 3.60 (s, 3 H) 3.92 (s, 3 H) 4.67 (br. s, 2 H) 6.63 (d, |
| | J = 9.23 Hz, 1 H) 6.76-6.92 (m, 2 H) 7.21 (d, J = 2.52 Hz, 1 H) 7.28-7.41 (m, |
| | 2 H) 7.78 (s, 1 H) |
| 31 | ¹H NMR (360 MHz, CHLOROFORM-d) δ ppm 2.54-2.86 (m, 3 H) 3.02 (d, |
| | J = 17.9 Hz, 1 H) 3.28 (d, J = 17.9 Hz, 1 H) 3.78-3.92 (m, 2 H) 3.95 (s, 3 H) |
| | 4.10 (br s, 2 H) 6.77-6.85 (m, 1 H) 6.89 (td, J = 8.2, 2.2 Hz, 1 H) 7.36-7.41 |
| | (m, 1 H) 7.51 (td, J = 9.1, 6.6 Hz, 1 H) 7.65 (dt, J = 7 .7 , 2.0 Hz, 1 H) 7.85 (br s, |
| | 1 H) 8.60 (dd, J = 2.2, 0.7 Hz, 1 H) 8.63 (dd, J = 4.8, 1.8 Hz, 1 H) |
| 32 | ¹H NMR (360 MHz, CHLOROFORM-d) δ ppm 2.11 (d, J = 12.8 Hz, 3 H) 2.25- |
| | 2.38 (m, 1 H) 2.44-2.54 (m, 1 H) 2.83 (q, J = 9.5 Hz, 1 H) 3.04 (dd, J = 17.9, |
| | 4.4 Hz, 1 H) 3.24-3.32 (m, 1 H) 3.74-3.84 (m, 1 H) 3.84-3.92 (m, 1 H) |
| | 3.96 (s, 3 H) 6.77-6.86 (m, 1 H) 6.89 (br t, J = 8.2 Hz, 1 H) 7.08 (d, J = 4.8 Hz, |
| | 1 H) 7.44-7.54 (m, 1 H) 7.73 (d, J = 3.3 Hz, 1 H) 8.50 (d, J = 4.9 Hz, 1 H) 8.56 |
| | (d, J = 2.9 Hz, 1 H) |
| 112 | ¹H NMR (360 MHz, DMSO-d₆) δ ppm 2.03 (d, J = 30.4 Hz, 3 H) 2.22 (d, |
| | J = 27.4 Hz, 3 H) 2.29-2.44 (m, 1 H) 2.53-2.61 (m, 1 H) 2.72 (dd, J = 11.9, 4.2 |
| | Hz, 1 H) 2.79-2.89 (m, 2 H) 2.90-3.00 (m, 1 H) 3.04 (br d, J = 16.8 Hz, 1 H) |
| | 3.85 (s, 3 H) 6.25 (br d, J = 4.8 Hz, 2 H) 7.07-7.15 (m, 1 H) 7.26 (ddd, J = 12.3, |
| | 9.3, 2.6 Hz, 1 H) 7.30-7.39 (m, 1 H) 7.77 (d, J = 9.1 Hz, 1 H) |

### Pharmacological Examples

The compounds provided in the present invention are inhibitors of the beta-site APP-cleaving enzyme 1 (BACE1). Inhibition of BACE1, an aspartic protease, is believed to be relevant for treatment of Alzheimer's Disease (AD). The production and accumulation of beta-amyloid peptides (Abeta) from the beta-amyloid precursor protein (APP) is believed to play a key role in the onset and progression of AD. Abeta is produced from the amyloid precursor protein (APP) by sequential cleavage at the N- and C-termini of the Abeta domain by beta-secretase and gamma-secretase, respectively.

Compounds of Formula (I) are expected to have their effect substantially at BACE1 by virtue of their ability to inhibit the enzymatic activity. The behaviour of such inhibitors tested using a biochemical Fluorescence Resonance Energy Transfer (FRET) based assay and a cellular αLisa assay in SKNBE2 cells described below and which are suitable for the identification of such compounds, and more particularly the compounds according to Formula (I), are shown in Table 8 and Table 9.

### BACE1 Biochemical Fret Based Assay

This assay is a Fluorescence Resonance Energy Transfer Assay (FRET) based assay. The substrate for this assay is an APP derived 13 amino acids peptide that contains the ‘Swedish’ Lys-Met/Asn-Leu mutation of the amyloid precursor protein (APP) beta-secretase cleavage site. This substrate also contains two fluorophores: (7-methoxycoumarin-4-yl) acetic acid (Mca) is a fluorescent donor with excitation wavelength at 320 nm and emission at 405 nm and 2,4-Dinitrophenyl (Dnp) is a proprietary quencher acceptor. The distance between those two groups has been selected so that upon light excitation, the donor fluorescence energy is significantly quenched by the acceptor, through resonance energy transfer. Upon cleavage by BACE1, the fluorophore Mca is separated from the quenching group Dnp, restoring the full fluorescence yield of the donor. The increase in fluorescence is linearly related to the rate of proteolysis.

Briefly in a 384-well format recombinant BACE1 protein in a final concentration of 0.04 μg/ml is incubated for 450 minutes at room temperature with 20 μM substrate in incubation buffer (50 mM Citrate buffer pH 5.0, 0.05% PEG) in the presence of compound or DMSO. Next the amount of proteolysis is directly measured by fluorescence measurement (excitation at 320 nm and emission at 405 nm) at different incubation times (0, 30, 60, 90, 120 and 450 min). For every experiment a time curve (every 30 min between 0 min and 120 min) is used to determine the time where we find the lowest basal signal of the high control. The signal at this time (Tx) is used to subtract from the signal at 450 min. Results are expressed in RFU, as difference between T450 and Tx.

A best-fit curve is fitted by a minimum sum of squares method to the plot of % Controlmin versus compound concentration. From this an IC₅₀ value (inhibitory concentration causing 50% inhibition of activity) can be obtained.
LC=Median of the low control values=Low control: Reaction without enzyme
HC=Median of the High control values=High Control: Reaction with enzyme

% Effect=100−[(sample−LC)/(HC−LC)*100]

% Control=(sample/HC)*100

% Controlmin=(sample−LC)/(HC−LC)*100

The following exemplified compounds were tested essentially as described above and exhibited the following the activity:

TABLE 8

| | BIOCHEMICAL FRET |
|---|---|
| | BASED ASSAY |
| CO. NO. | pIC₅₀ |
| 1 | 6.4 |
| 2 | 6.8 |
| 3 | 5.06 |
| 4 | 6.78 |
| 5 | 6.79 |
| 6 | 6.63 |
| 7 | 6.84 |
| 8 | 6.66 |
| 9 | 6.88 |
| 10 | 7.45 |
| 11 | 7.66 |
| 12 | 7.04 |
| 13 | 7.15 |
| 15 | 7.52 |
| 16 | 7.26 |
| 17 | 7.51 |
| 19 | 6.79 |
| 20 | 7.61 |
| 21 | 6.87 |
| 22 | 6.75 |
| 23 | 7.12 |
| 24 | 7.86 |
| 25 | 8.23 |
| 26 | 7.6 |
| 27 | 7.93 |
| 28 | 6.84 |
| 29 | 7.45 |
| 30 | 6.16 |
| 31 | 7.16 |
| 32 | 7.3 |
| 33 | 6.85 |
| 34 | 5.22 |
| 35 | 7.46 |
| 36 | 6.86 |
| 37 | 7.37 |
| 38 | 7.81 |
| 39 | 8.3 |
| 41 | 7.57 |
| 42 | 7.56 |
| 43 | 7.43 |
| 44 | 7.91 |
| 45 | 8.56 |
| 46 | 8.64 |
| 47 | 7.99 |
| 49 | 8.81 |
| 52 | 8.38 |
| 53 | 7.97 |
| 55 | 8.45 |
| 60 | 8.26 |
| 66 | 8.22 |
| 76 | 8.22 |
| 77 | 8.35 |
| 79 | 8.17 |
| 80 | 9.01 |
| 81 | 7.3 |
| 82 | 6.19 |
| 87 | 8.42 |
| 88 | 8.08 |
| 89 | 7.88 |
| 90 | 7.06 |
| 91 | 7.27 |
| 92 | 8.15 |
| 93 | 7.25 |
| 94 | 7.79 |
| 95 | 7.94 |
| 102 | 8.16 |
| 103 | 6.68 |
| 104 | 7.95 |
| 105 | 7.44 |
| 106 | 8.06 |
| 107 | 8.5 |
| 108 | 8.11 |
| 109 | 7.81 |
| 110 | 6.69 |
| 111 | 7.76 |
| 112 | 7.83 |
| 57 | 6.6 |
| 134 | 8.06 |
| 135 | 7.6 |
| 136 | 7.83 |
| 137 | 7.97 |
| 151a | 7.44 |
| 171a | 6.94 |
| 174a | 7.86 |
| 180a | 5.91 |
| 181a | 7.14 |
| 182 | 5.32 |
| 183 | 5.35 |
| 184 | <5 |
| 185 | <5 |
| 186 | <5 |
| 189 | <5 |
| 193 | 5.26 |
| 194 | <5 |
| 195 | 6.9 |
| 196 | <5 |
| 197 | 7.02 |
| 198 | 5.97 |
| 199 | 7.79 |
| 200 | 7.75 |
| 201 | 5.19 |

### Cellular αLisa Assay in SKNBE2 Cells

In two αLisa assays the levels of Abeta total and Abeta 1-42 produced and secreted into the medium of human neuroblastoma SKNBE2 cells are quantified. The assay is based on the human neuroblastoma SKNBE2 expressing the wild type Amyloid Precursor Protein (hAPP695). The compounds are diluted and added to these cells, incubated for 18 hours and then measurements of Abeta 1-42 and Abeta total are taken. Abeta total and Abeta 1-42 are measured by sandwich αLisa. αLisa is a sandwich assay using biotinylated antibody AbN/25 attached to streptavidin coated beads and antibody Ab4G8 or cAb42/26 conjugated acceptor beads for the detection of Abeta total and Abeta 1-42 respectively. In the presence of Abeta total or Abeta 1-42, the beads come into close proximity. The excitation of the donor beads provokes the release of singlet oxygen molecules that trigger a cascade of energy transfer in the acceptor beads, resulting in light emission. Light emission is measured after 1 hour incubation (excitation at 650 nm and emission at 615 nm).

A best-fit curve is fitted by a minimum sum of squares method to the plot of % Controlmin versus compound concentration. From this an IC₅₀ value (inhibitory concentration causing 50% inhibition of activity) can be obtained.
LC=Median of the low control values=Low control: cells preincubated without compound, without biotinylated Ab in the αLisa
HC=Median of the High control values=High Control: cells preincubated without compound

% Effect=100−[(sample−LC)/(HC−LC)*100]

% Control=(sample/HC)*100

% Controlmin=(sample−LC)/(HC−LC)*100

The following exemplified compounds were tested essentially as described above and exhibited the following the activity:

TABLE 9

| | CELLULAR αLISA ASSAY | CELLULAR αLISA ASSAY |
|---|---|---|
| | IN SKNBE2 CELLS | IN SKNBE2 CELLS |
| | ABETA 42 | ABETA TOTAL |
| CO. NO. | PIC50 | pIC₅₀ |
| 1 | 7.16 | 7.13 |
| 2 | 7.27 | 7.33 |
| 3 | 5.39 | 5.33 |
| 4 | 6.73 | n.t. |
| 5 | 7.58 | 7.49 |
| 6 | 6.7 | nt. |
| 7 | 7.15 | nt. |
| 8 | 6.87 | nt. |
| 9 | 7.68 | nt. |
| 10 | 8.07 | nt. |
| 11 | 8.53 | nt. |
| 12 | 7.65 | nt. |
| 13 | 8.84 | nt. |
| 15 | 7.76 | nt. |
| 16 | 6.86 | 6.86 |
| 17 | 6.88 | 6.84 |
| 18 | | nt. |
| 19 | 6.38 | nt. |
| 20 | 7.63 | 7.63 |
| 21 | 6.45 | 6.52 |
| 22 | 7.38 | 7.31 |
| 23 | 7.58 | 7.53 |
| 24 | 8.16 | 8.18 |
| 25 | 8.77 | 8.72 |
| 26 | 7.37 | nt. |
| 27 | 8.41 | nt. |
| 28 | 7.38 | 7.32 |
| 29 | 7.87 | 7.9 |
| 30 | 6.77 | nt. |
| 31 | 8.05 | nt. |
| 32 | 8.14 | nt. |
| 33 | 7.79 | nt. |
| 34 | 5.85 | 5.82 |
| 35 | 7.86 | 7.92 |
| 36 | 7.78 | 7.8 |
| 37 | 7.77 | nt. |
| 38 | 8.45 | n.t. |
| 39 | 8.78 | 8.71 |
| 41 | 7.35 | n.t. |
| 42 | 6.81 | n.t. |
| 43 | 8 | n.t. |
| 44 | 8.6 | n.t. |
| 45 | 8.65 | 8.68 |
| 46 | 9.21 | n.t. |
| 47 | 8.25 | n.t. |
| 49 | 8.56 | 8.75 |
| 52 | 7.96 | 7.88 |
| 53 | 7.84 | 7.9 |
| 55 | 7.99 | n.t. |
| 60 | 7.25 | n.t. |
| 66 | 7.64 | n.t. |
| 76 | 8.39 | n.t. |
| 77 | 8.26 | 8.31 |
| 79 | 8.13 | 7.96 |
| 80 | 8.84 | n.t. |
| 81 | 7.22 | n.t. |
| 82 | 6.32 | n.t. |
| 87 | 9.19 | 9.2 |
| 88 | 8.74 | n.t. |
| 89 | 8.7 | 8.7 |
| 90 | 7.76 | n.t. |
| 91 | 7.83 | n.t. |
| 92 | 8.45 | 8.46 |
| 93 | 8.3 | 8.2 |
| 94 | 8.46 | 8.51 |
| 95 | 8.36 | 8.24 |
| 102 | 8.46 | n.t. |
| 103 | 7.86 | n.t. |
| 104 | 8.42 | n.t. |
| 105 | 8.19 | n.t. |
| 106 | 8.23 | n.t. |
| 107 | 9.04 | n.t. |
| 108 | 8.88 | n.t. |
| 109 | 8.25 | n.t. |
| 110 | 7.21 | n.t. |
| 111 | 8.01 | n.t. |
| 112 | 7.78 | n.t. |
| 57 | 7.44 | 7.37 |
| 134 | 7.6 | n.t. |
| 135 | 7.6 | 7.62 |
| 136 | 7.73 | n.t. |
| 137 | 7.74 | 7.82 |
| 151a | 7.1 | n.t. |
| 171a | 6.81 | n.t. |
| 174a | 6.89 | n.t. |
| 180a | 5.57 | n.t. |
| 181a | 6.4 | n.t. |
| 182 | 5.89 | 5.88 |
| 183 | 5.51 | 5.46 |
| 184 | 5.27 | n.t. |
| 185 | <5.05 | n.t. |
| 186 | <5.05 | n.t. |
| 189 | 5.16 | n.t. |
| 193 | 6.01 | 5.9 |
| 194 | <5.05 | 5.16 |
| 195 | 7.09 | 7.09 |
| 196 | <5.05 | <5.05 |
| 197 | 7.31 | 7.33 |
| 198 | 6.58 | n.t. |
| 199 | 8.36 | n.t. |
| 200 | 8.09 | 8.05 |
| 201 | 5.64 | 5.69 |

n.t. means not tested

### BACE2 Biochemical Fret Based Assay

This assay is a Fluorescence Resonance Energy Transfer Assay (FRET) based assay. The substrate for this assay contains the ‘Swedish’ Lys-Met/Asn-Leu mutation of the amyloid precursor protein (APP) beta-secretase cleavage site. This substrate also contains two fluorophores: (7-methoxycoumarin-4-yl) acetic acid (Mca) is a fluorescent donor with excitation wavelength at 320 nm and emission at 405 nm and 2,4-Dinitrophenyl (Dnp) is a proprietary quencher acceptor. The distance between those two groups has been selected so that upon light excitation, the donor fluorescence energy is significantly quenched by the acceptor, through resonance energy transfer. Upon cleavage by the beta-secretase, the fluorophore Mca is separated from the quenching group Dnp, restoring the full fluorescence yield of the donor. The increase in fluorescence is linearly related to the rate of proteolysis.

Briefly in a 384-well format recombinant BACE2 protein in a final concentration of 0.4 μg/ml is incubated for 450 minutes at room temperature with 10 μM substrate in incubation buffer (50 mM Citrate buffer pH 5.0, 0.05% PEG, no DMSO) in the absence or presence of compound. Next the amount of proteolysis is directly measured by fluorescence measurement at T=0 and T=450 (excitation at 320 nm and emission at 405 nm). Results are expressed in RFU (Relative Fluorescence Units), as difference between T450 and TO.

A best-fit curve is fitted by a minimum sum of squares method to the plot of % Controlmin versus compound concentration. From this an IC₅₀ value (inhibitory concentration causing 50% inhibition of activity) can be obtained.
LC=Median of the low control values=Low control: Reaction without enzyme
HC=Median of the High control values=High Control: Reaction with enzyme

% Effect=100−[(sample−LC)/(HC−LC)*100]

% Control=(sample/HC)*100

% Controlmin=(sample−LC)/(HC−LC)*100

The following exemplified compounds were tested essentially as described above and exhibited the following the activity:

TABLE 10

| | BIOCHEMICAL FRET |
|---|---|
| | BASED ASSAY |
| Co. NO. | pIC₅₀ |
| 1 | 6.33 |
| 2 | 6.76 |
| 3 | 5.02 |
| 4 | 6.48 |
| 5 | 7.2 |
| 6 | 6.66 |
| 7 | 6.81 |
| 8 | 6.27 |
| 9 | 6.66 |
| 10 | 6.34 |
| 11 | 6.66 |
| 12 | 6.6 |
| 13 | 7.4 |
| 15 | 6.94 |
| 16 | 7.11 |
| 17 | 7.17 |
| 19 | 6.55 |
| 20 | 7.85 |
| 21 | 7.01 |
| 22 | 6.62 |
| 23 | 6.81 |
| 24 | 7.91 |
| 25 | 7.92 |
| 26 | 6.32 |
| 27 | 5.4 |
| 28 | 5.92 |
| 29 | 7.45 |
| 30 | 5.94 |
| 31 | 6.75 |
| 32 | 5.95 |
| 33 | 5.21 |
| 34 | 5.22 |
| 35 | 7.35 |
| 36 | 7.34 |
| 37 | 6.91 |
| 38 | 6.64 |
| 39 | 5.78 |
| 41 | 7.32 |
| 42 | 7.53 |
| 43 | 6.7 |
| 44 | 7.55 |
| 45 | 8.56 |
| 46 | 8.44 |
| 47 | 8.18 |
| 49 | 8.43 |
| 52 | 7.35 |
| 53 | 6.84 |
| 55 | 7.4 |
| 60 | 6.9 |
| 66 | 7.45 |
| 76 | 8.06 |
| 77 | 7.27 |
| 79 | 6.4 |
| 80 | 8.6 |
| 81 | 6.3 |
| 82 | 5.34 |
| 87 | 8.15 |
| 88 | 7.91 |
| 89 | 6.67 |
| 90 | 6.78 |
| 91 | 6.34 |
| 92 | 7.03 |
| 93 | 6.08 |
| 94 | 6.56 |
| 95 | 6.65 |
| 102 | 7.86 |
| 103 | 5.59 |
| 104 | 7.69 |
| 105 | 5.83 |
| 106 | 6.71 |
| 107 | 7.16 |
| 109 | 7.32 |
| 110 | 5.23 |
| 111 | 6.31 |
| 112 | 5.76 |
| 57 | <5 |
| 134 | 7.91 |
| 135 | 7.55 |
| 136 | 7.83 |
| 137 | 7.72 |
| 151a | 6.65 |
| 171a | 5.2 |
| 174a | 6.48 |
| 180a | 5.69 |
| 181a | 5.24 |
| 182 | 5.21 |
| 183 | 5.16 |
| 184 | <5 |
| 185 | <5 |
| 186 | <5 |
| 189 | <5 |
| 193 | <5 |
| 194 | <5 |
| 195 | 6.85 |
| 196 | <5 |
| 197 | 7 |
| 198 | 6.3 |
| 199 | 8.15 |
| 200 | 6.36 |
| 201 | <5 |

### Biochemical Assay—Automated

### General Methods

Unless otherwise indicated all biochemicals were purchased from Sigma-Aldrich Chemical Company, Poole, Dorset, U.K. and non-aqueous solvents, of analytical or higher grade, were purchased from ThermoFisher Scientific, Loughborough, U.K. MilliQ water (Elix 5 & MilliQ Gradient; Merck Millipore) was used as the base aqueous solvent to make up the biological buffers. Base assay buffer was prepared by adding a 50 mM solution of citric acid (1.00244; Merck Biosciences) to stirring solution of 50 mM trisodium citrate (1.06448; Merck Biosciences) until a final pH of 5.0 was achieved. To this was added a 40% solution of polyethylene glycol (“PEG”) (P1458; Sigma Aldrich) to a final concentration of 0.05%; hence base buffer comprised of 50 mM sodium citrate, pH 5.0 containing 0.05% PEG. All assays were routinely carried out in 384-well assay plates (Costar 4514; Corning Life Sciences) and incubated at 37±1° C. for 60 min. prior to reading the endpoint fluorescence intensity. The (7-methoxyl coumarin-4-yl)acetic acid based substrate β-secretase substrate VI (M2465; Bachem) was prepared as a 1 mM stock in 100% DMSO (D/4121/PB08; ThermoFisher). Assay buffer was prepared by adding DMSO to base buffer to a final concentration of 1% (vol./vol.). β-secretase I (18.64 μM; “BACE1”) and β-secretase II (4.65 μM; “BACE2”) were obtained from Janssen Pharmaceutica, Beerse, Belgium and were stored as frozen aliquots (˜20 μl) and thawed as required.

### Manual Assays

Typically 12.5 μl of assay buffer was dispensed to rows B to P of the assay plate. To row A was added 18.75 μl of test compound diluted appropriately in assay buffer. A 6.25 μl aliquot of sample was transferred from row A to row B and the sample mixed three times by pipette. The process was repeated down the plate and 6.25 μl of solution discarded at row N post-mix. Rows O and P were designated as the positive and negative controls. To row P was added 6.25 μl base buffer. To rows A to O was added 6.25 μl enzyme (freshly prepared 40 nM BACE1 or 40 nM BACE2) diluted in base buffer. To initiate the assay 6.25 μl of freshly prepared 80 μM substrate, made up by diluting the 1 mM in 100% DMSO solution into HPLC grade water (Optima W6-212; ThermoFisher), was added to all the wells. The assay plate was covered and incubated at 37±1° C. for 60 min. The fluorescence intensity of the wells was read at 360/405 nm (excitation/emission) utilising a nine reads per well protocol (50 ms integration; density of 3, 0.25 mm spacing; SpectraMAX Paradigm plate reader; TUNE cartridge; SoftMax Pro v 6.3 software; Molecular Devices UK Ltd., Wokingham, Berkshire, UK) and outputting the median value of the nine reads as a text file. Data analysis was carried out using Prism software v 6.3 (GraphPad Inc., San Diego, Calif., USA) using the non-linear regression analysis models supplied by the vendor. For IC₅₀ determinations the four parameter logistic variable slope model was used to fit the raw fluorescence intensity data with the ‘bottom’ fixed to the negative control.

### Automated Bioassay Hardware

The CyclOps bioassay module consisted of a fraction collection station, a reagent station, liquid handling robotics, plate store and an integrated plate reader (SpectraMAX Paradigm, TUNE cartridge, SoftMax Pro v 6.3; Molecular Devices). The fraction collection station composed of a 384 well collection plate (P-384-240SQ-C; Axygen, Union City, Calif., USA) mounted on a H-portal carriage (Festo AG & Co. KG, Esslingen, Germany), a syringe drive and a two-way six port injection valve fitted with a 200 μl loop (VICI AG International, Schenkon Switzerland). The output of the injection valve was addressable to all the positions of a 384 well collection plate. The reagent station consisted of hydraulically cooled (10-12° C.) aluminium segments; each manufactured to house a SBS microtiter plate footprint. Independent addressable reagent stations were housed within these sections. Where required, custom aluminium housings were used to accommodate standard laboratory plastic ware (e.g. Eppendorf tubes, Falcon tubes, etc.). As and when required the reagent reservoirs were covered and the lids contained holes through which the Teflon-coated probe could access solutions. The reagents present on the liquid handling system were:
Probe wash solution (˜150 ml; 33.3:33.3:33.3 water:propan-2-ol (P/7508/17; ThermoFisher):methanol (M/4058/17; ThermoFisher) contained in a covered reagent reservoir (390007; Porvair Sciences Ltd., Leatherhead, UK).
Assay buffer solution
HPLC grade water
40 nM BACE1 diluted in base buffer contained in a 5 ml Eppendorf tube (0030 119.401; Eppendorf)
400 nM BACE2 diluted in 25 mM tris (648311; Merck Biosciences, Nottingham, U.K.), pH 7.5 containing 100 mM sodium chloride and 20% glycerol (16374; USB Corp., Cleveland, Ohio, USA) contained in a 1.5 ml Eppendorf tube (0030 000.919; Eppendorf)
1 mM substrate in 100% DMSO contained in a 1.5 ml Eppendorf tube (maintained at ambient temperature)
Two empty 1.5 ml Eppendorf tubes

The liquid handling system composed of a LISSY system (Zinsser Analytik GmbH, Frankfurt, Germany) equipped with gripper arm and single teflon-coated stainless steel probe. Between every liquid handling step the teflon-coated stainless steel probe was washed with probe wash solution followed by system liquid (water). Control of the bioassay system was achieved using WinLISSY software (Zinsser Analytik) and SoftMax Pro (which was under WinLISSY automation command control). A plate store housed a stack of assay plates (Costar 4514). Input and output relays enabled contact closure control and feedback between the bioassay module and the CyclOps control software. The plate store was an aluminium rack that accommodated a stack of assay plates which could be accessed by the liquid handling system.

### The Automated Bioassay Process

The output of the dilution module flowed through the collection station injection valve set in the ‘load’ position. With WinLISSY set to input polling mode contact closure by the CyclOps control software initiated the bioassay protocol. The first action triggered the injection valve to the ‘inject’ position, isolating the loop contents, and the fraction collection system dispensed the loop contents to an addressable well on the collection plate. Concomitantly the liquid handling system delivered an assay plate to an assay station on the liquid handling bed. Onto columns of the assay plate the liquid handling system dispensed 12.5 μl assay buffer down two columns of the assay plate from row B to row P. To row A was added 18.75 μl of test compound from the respective well of the collection plate. A 6.25 μl aliquot of sample from row A was transferred to row B. The process was repeated down the plate for both columns and 6.25 μl reagent discarded at row N. Rows O and P were designated as the positive and negative controls. To row P was added 6.25 μl assay buffer. To rows A to O of the first column was added 6.25 μl 40 nM BACE1 stored in base buffer. For the BACE2 enzyme addition, 17.5 μl of 400 nM BACE2 was diluted with 157.5 μl base buffer. This was mixed by pipetting 175 μl of solution five times in the designated receiving Eppendorf tube and then 6.25 μl of the diluted BACE2 was added up the respective column. For the MCA substrate, 30.8 μl of 1 mM MCA substrate in 100% DMSO was diluted with 385 μl HPLC water. This was mixed by pipetting 400 μl five times in the designated receiving Eppendorf tube and 6.25 μl added up the respective columns. The assay plate was then transferred to the plate reader carriage, the drawer closed and the assay incubation initiated. After 60 min. WinLISSY executed a sub-routine that instructed the plate reader to load and execute a protocol file which read the fluorescence intensity. This protocol file contained the parameters required to read the microtiter plate and write the corresponding data as a text file. Fluorescence intensity was read at 360/405 nm (excitation/emission) utilising a nine reads per well protocol (50 ms integration; density of 3, 0.25 mm spacing) and outputted the median value of the nine reads as a text file.

### CyclOps Bioassay Data Analysis

CyclOps software was set to poll the bioassay shared data file folder. On saving the data, WinLISSY sent an output contact closure signal notifying the CyclOps software that the bioassay had been completed. CyclOps software opened, processed and analysed the data. Data processing consisted of appending the respective concentration of test article to the corresponding rows (with data received from the dilution module). Thereafter the data was analysed (MATLAB; MathWorks, Cambridge, U.K.) by a non-linear regression analysis employing a four parameter logistic model to determine the IC₅₀. The span was fixed between baseline (i.e. row P) and the maximum observed positive control rate (i.e. row O). To maintain data quality, rules were set up to govern automated bioassay data analysis. In the first instance if no less than seventy-five percent activity or no greater than twenty-five percent activity were observed the data was rejected. This ensured that there was sufficient titration data for good analysis to be carried out. Thereafter the quality of the fit was judged by the R-squared value. If this value fell below 0.85 then the data was rejected. In all cases rejection led to a bioassay failure tag being reported to the system. Outlier analysis was carried out as described previously (Motulsky, H. J. and Brown, R. E., (2006), BMC Bioinformatics, 7, 123) with a Q value of 10%. For the automated IC₅₀ analysis a maximum of three outliers could be excluded prior to an error of fit flag being generated. Cross validation of the bioassay data was achieved by analysing the same using Prism software v 6.3 (GraphPad Inc.) employing a non-linear regression analysis four parameter logistic variable slope model to fit the raw fluorescence intensity data with the ‘bottom’ fixed to the negative control.

TABLE 11

| | AUTOMATED | AUTOMATED |
|---|---|---|
| | ASSAY BACE1 | ASSAY BACE2 |
| Co. NO. | pIC₅₀ | pIC₅₀ |
| 47 | 8.01 | 8.23 |
| 48 | 7.76 | 7.63 |
| 49 | 8.6 | 9.02 |
| 50 | 8.28 | 7.96 |
| 51 | 7.34 | 6.83 |
| 54 | 8.54 | 8.42 |
| 55 | 7.77 | 7.3 |
| 56 | 8.11 | 7.63 |
| 57 | 5.87 | 5.74 |
| 58 | 7.77 | 6.99 |
| 59 | 7.68 | 7.15 |
| 60 | 7.65 | 6.73 |
| 62 | 8.77 | 8.08 |
| 64 | 6.15 | 5.2 |
| 65 | 7.84 | 7.29 |
| 66 | 7.5 | 6.68 |
| 67 | 7.64 | 7.15 |
| 70 | 6.88 | 5.76 |
| 71 | 6.41 | 5.66 |
| 72 | 7.99 | 7.11 |
| 73 | 6.55 | 5.47 |
| 74 | 7.52 | 6.72 |
| 78 | 7.85 | 6.75 |
| 81 | 7.24 | 6.24 |
| 83 | 6.59 | 6.06 |
| 84 | 8.28 | 8.51 |
| 86 | 8.44 | 8.33 |
| 96 | 7.2 | 6.41 |
| 97 | 7.96 | 7.6 |
| 98 | 7.66 | 7.38 |
| 99 | 7.27 | 6.99 |
| 100 | 7.68 | 7.31 |
| 106 | 8.33 | 7.12 |
| 112 | 7.795 | 6.18 |
| 113 | 8.41 | 8.92 |
| 114 | 8.52 | 8.64 |
| 115 | 7.92 | 7.57 |
| 116 | 7.6 | 6.28 |
| 117 | 7.68 | 6.34 |
| 119 | 6.77 | 6.17 |
| 122 | 7.62 | 7.3 |
| 123 | 7.59 | 6.81 |
| 124 | 7.4 | 6.64 |
| 125 | 7.13 | 6.59 |
| 126 | 8.05 | 7.18 |
| 127 | 6.77 | 5.6 |
| 128 | 7.01 | 6.19 |
| 130 | 7.4 | 7.03 |
| 131 | 6.84 | 5.57 |
| 132 | 7.82 | 7.16 |
| 133 | 8.6 | 7.67 |
| 138 | 7.21 | 7.49 |
| 139 | 5.55 | 4.97 |
| 140 | 5.52 | 5.52 |
| 141 | 6.24 | 6.24 |
| 142 | 8.4 | 7.28 |
| 143 | 6.43 | 4.78 |
| 144 | 7.42 | 6.43 |
| 145 | 6.89 | 5.52 |
| 146 | 6.74 | 6.63 |
| 147 | 7.7 | 6.64 |
| 148 | 6 | 5.19 |
| 149 | 5.09 | 5.17 |
| 150 | 6.12 | 5.3 |
| 151 | 7.77 | 6.02 |
| 152 | 7.52 | 5.65 |
| 153 | 6.73 | 6.64 |
| 154 | 7.51 | 7.88 |
| 155 | 7.77 | 6.12 |
| 156 | 6.32 | 6.36 |
| 157 | 7.1 | 6.13 |
| 158 | 6.6 | 5.49 |
| 159 | 7.37 | 6.29 |
| 160 | 5.39 | 4.82 |
| 161 | 9 | 8.4 |
| 162 | 6.85 | 5.96 |
| 163 | 7.29 | 7.2 |
| 164 | 8.3 | 8.22 |
| 165 | 7.62 | 6.61 |
| 166 | 7.54 | 6.79 |
| 167 | 7.21 | 7.6 |
| 168 | 7.6 | 7.54 |
| 169 | 7.33 | 7.16 |
| 170 | 6.88 | 5.6 |
| 171 | 7.07 | 6.21 |
| 172 | 6.53 | 5.34 |
| 173 | 7.77 | 7.03 |
| 174 | 8.26 | 6.29 |
| 175 | 9 | 6.56 |
| 177 | 8.05 | 6.84 |
| 178 | 8.22 | 7.49 |
| 179 | 6.47 | 4.86 |
| 180 | 7.72 | 6.19 |
| 181 | 7.215 | 4.893 |

### Demonstration of In Vivo Efficacy

Aβ lowering agents of the invention can be used to treat AD in mammals such as humans or alternatively demonstrating efficacy in animal models such as, but not limited to, the mouse, rat, or guinea pig. The mammal may not be diagnosed with AD, or may not have a genetic predisposition for AD, but may be transgenic such that it overproduces and eventually deposits Aβ in a manner similar to that seen in humans afflicted with AD.

Aβ lowering agents can be administered in any standard form using any standard method. For example, but not limited to, Aβ lowering agents can be in the form of liquid, tablets or capsules that are taken orally or by injection. Aβ lowering agents can be administered at any dose that is sufficient to significantly reduce levels of Aβ in the blood, blood plasma, serum, cerebrospinal fluid (CSF), or brain.

To determine whether acute administration of an Aβ lowering agent would reduce Aβ levels in vivo, non-transgenic rodents, e.g. mice or rats were used. Animals treated with the Aβ lowering agent were examined and compared to those untreated or treated with vehicle and brain levels of soluble Aβ42, Aβ40, Aβ38, and Aβ37 were quantitated by Meso Scale Discovery's (MSD) electrochemiluminescence detection technology. Treatment periods varied from hours (h) to days and were adjusted based on the results of the Aβ lowering once a time course of onset of effect could be established.

A typical protocol for measuring Aβ lowering in vivo is shown but it is only one of many variations that could be used to optimize the levels of detectable Aβ. For example, Aβ lowering compounds were formulated in 20% of Captisol® (a sulfo-butyl ether of β-cyclodextrin) in water or 20% hydroxypropyl 3 cyclodextrin. The Aβ lowering agents were administered as a single oral dose or by any acceptable route of administration to overnight fasted animals. After 4 h, the animals were sacrificed and Aβ levels were analysed.

Blood was collected by decapitation and exsanguinations in EDTA-treated collection tubes. Blood was centrifuged at 1900 g for 10 minutes (min) at 4° C. and the plasma recovered and flash frozen for later analysis. The brain was removed from the cranium and hindbrain. The cerebellum was removed and the left and right hemisphere were separated. The left hemisphere was stored at −18° C. for quantitative analysis of test compound levels. The right hemisphere was rinsed with phosphate-buffered saline (PBS) buffer and immediately frozen on dry ice and stored at −80° C. until homogenization for biochemical assays.

Mouse brains from non-transgenic animals were resuspended in 8 volumes of 0.4% DEA (diethylamine)/50 mM NaCl containing protease inhibitors (Roche-11873580001 or 04693159001) per gram of tissue, e.g. for 0.158 g brain, add 1.264 ml of 0.4% DEA. All samples were homogenized in the FastPrep-24 system (MP Biomedicals) using lysing matrix D (MPBio #6913-100) at 6 m/s for 20 seconds. Homogenates were centrifuged at 20800×g for 5 min and supernatants collected. Supernatants were centrifuged at 221.300×g for 50 min. The resulting high speed supernatants were then transferred to fresh eppendorf tubes. Nine parts of supernatant were neutralized with 1 part 0.5 M Tris-HCl pH 6.8 and used to quantify Aβ.

To quantify the amount of Aβ42, Aβ40, Aβ38, and Aβ37 in the soluble fraction of the brain homogenates, simultaneous specific detection of Aβ42, Aβ40, Aβ38, and Aβ37 was performed using MSD's electro-chemiluminescence multiplex detection technology. In this assay purified monoclonal antibodies specific for Abeta37 (JRD/Aβ37/3), Abeta38 (J&JPRD/Aβ38/5), Abeta40 (JRF/cAβ40/28), and Abeta42 (JRF/cAβ42/26) were coated on MSD 4-plex plates. Briefly, the standards (a dilution of synthetic Aβ42, Aβ40, Aβ38, and Aβ37) were prepared in 1.5 ml Eppendorf tube in Ultraculture, with final concentrations ranging from 10000 to 0.3 pg/m. The samples and standards were co-incubated with Sulfo-tag labelled JRF/rAβ/2 antibody to the N-terminus of Aβ as detector antibody. 50 μl of conjugate/sample or conjugate/standards mixtures were then added to the antibody-coated plate. The plate was allowed to incubate overnight at 4° C. in order to allow formation of the antibody-amyloid complex. Following this incubation and subsequent wash steps the assay was finished by adding read buffer according to the manufacturer's instructions (Meso Scale Discovery, Gaitherburg, Md.).

The SULFO-TAG emits light upon electrochemical stimulation initiated at the electrode. MSD Sector instrument SI6000 was used for signal read-out.

In this model a AB lowering compared to untreated animals would be advantageous, in particular a AB lowering with at least 10%, more in particular a AB lowering with at least 20%.

### Results

The results are shown in Table 12 (value for untreated animals as control (Ctrl) was set at 100):

TABLE 12

| | | | | | TIME |
|---|---|---|---|---|---|
| | | | | ROUTE OF | AFTER |
| CO. | Aβ40 (% vs | Aβ42 (% vs | DOSE | ADMINI- | ADMINI- |
| NO. | Ctrl)_Mean | Ctrl)_Mean | (mg/kg) | STRATION | STRATION |
| 22 | 39 | 32 | 10 | s.c. | 4 h |

s.c. means subcutaneous;
p.o. means oral

1. A compound of Formula (I) or (II) 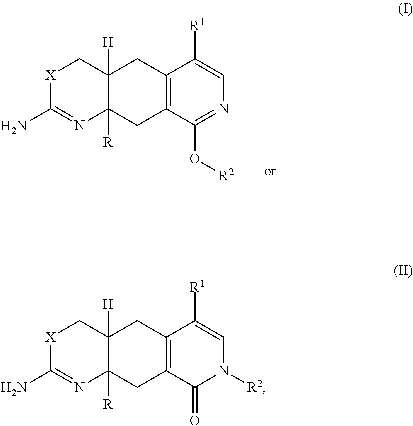
or a tautomer or a stereoisomeric form thereof, wherein
X is S or O;
R is phenyl optionally substituted with 1, 2 or 3 substituents each independently selected from the group consisting of halo; hydroxyl; C₁₋₃alkyl; cyano; nitro; Het; Ar; (C₁₋₃alkyloxy)C₁₋₃alkyl-NH—C₁₋₃alkyl-; C₁₋₆alkyloxy optionally substituted with cyano, or C₁₋₃alkyloxy; C₂₋₆alkynyloxy; tetrahydro-2H-pyranyloxy; Ar-oxy-; Het-oxy-;
Ar—CH(OH)—; —NR^{a}R^{b}; a divalent —NH—CH₂CH₂—O— substituent optionally substituted with 1 or 2 substituents each independently selected from halo and oxo;
C₁₋₄alkyl(C═O)—; Ar(C═O)—; and R³—C₁₋₆alkyloxy-; wherein
Het is selected from pyridinyl and pyrimidinyl, each of which can be optionally substituted with halo, cyano, C₁₋₃alkyl, C₁₋₃alkyloxy, —CF₃, and —OCF₃;
Ar is phenyl optionally substituted with halo, cyano, C₁₋₃alkyl, C₁₋₃alkyloxy, —CF₃, —OCF₃;
R^{a} is selected from H, or C₁₋₃alkyl; and
R^{b} is selected from C₁₋₃alkyl, (C₁₋₃alkyloxy)C₁₋₃alkyl(C═O)—, or Het¹(C═O)—;
R³ is selected from the group consisting of C₃₋₆cycloalkyl; Het¹; Ar¹; tetrahydro-2H-pyranyl; C₃₋₆cycloalkyloxy; tetrahydro-2H-pyranyloxy; Het¹-oxy-; and Ar¹-oxy-; wherein
Ar¹ is phenyl optionally substituted with halo, cyano, C₁₋₃alkyl, C₁₋₃alkyloxy, cyano-C₁₋₃alkyloxy —CF₃, or —OCF₃;
Het¹ is selected from the group consisting of pyridyl, pyrimidinyl, pyrazinyl, pyridazinyl, thienyl, pyrazolyl, isoxazolyl, 1H-imidazolyl, thiazolyl, oxazolyl,
1H-indolyl, and 1H-indazolyl; each of which is optionally substituted with 1 or 2 substituents each independently selected from the group consisting of halo, cyano,
C₁₋₃alkyl, C₁₋₃alkyloxy, —CF₃, and —OCF₃;
R¹ is selected from the group consisting of hydrogen; halo; cyano; C₁₋₃alkyl optionally substituted with hydroxyl or C₁₋₃alkyloxy; C₃₋₆cycloalkyl; C₃₋₆cycloalkenyl; (C₃₋₆cycloalkyl)C₁₋₃alkyl; C₁₋₃alkyloxy; —NR^{x}R^{y}; C₁₋₃alkyloxy-(C═O)—; C₁₋₃alkyloxy-C₂₋₃alkenyl; (halo-phenyl)-C₂₋₃alkenyl-; heterocyclyl; homoaryl; heteroaryl;
C₃₋₆cycloalkyloxy; homoaryloxy; heteroaryloxy; homoaryl-CH₂-oxy; and heteroaryl-CH₂-oxy;
wherein
R^{x} is hydrogen or C₁₋₃alkyl;
R^{y} is C₁₋₃alkyl or phenyl optionally substituted 1, 2, or 3 substituents each independently selected from halo, C₁₋₃alkyl, and C₁₋₃alkyloxy;
heterocyclyl is selected from the group consisting of piperidinyl, morpholinyl, 3,4-dihydro-2H-pyranyl; and tetrahydro-2H-pyranyl, each of which being optionally substituted with 1, 2 or 3 substituents each independently selected from the group consisting of C₁₋₃alkyl, C₃₋₆cycloalkyl and oxo;
homoaryl is phenyl optionally substituted with 1, 2 or 3 substituents each independently selected from the group consisting of halo, hydroxyl, cyano, C₁₋₃alkyl, mono-halo-C₁₋₃alkyl, poly-halo-C₁₋₃alkyl, cyano-C₁₋₃alkyl, C₁₋₃alkyloxy, mono-halo-C₁₋₃alkyloxy, poly-halo-C₁₋₃alkyloxy, C₁₋₃alkyloxy-(C═O)—, phenyloxy-, NR^{1a}R^{1b}, —(C═O)NR^{1a}R^{1b}, 1H-pyrazolyl optionally substituted with 1 or 2 methyl substituents; or is naphthalenyl, optionally substituted with C₁₋₃alkyl or C₁₋₃alkyloxy; wherein R^{1a} is hydrogen or
C₁₋₃alkyl and R^{1b} is C₁₋₃alkyl, or NR^{1a}R^{1b} form together a 1-pyrrolidinyl, 1-piperidinyl, 4-piperazinyl or a 4-morpholinyl;
heteroaryl is selected from the group consisting of pyridyl, 2-oxo-1,2-dihydropyridinyl, 6-oxo-1,6-dihydropyridinyl, pyrimidinyl, pyrazinyl, pyridazinyl, pyrazolyl, isoxazolyl, oxazolyl, thiophenyl, indolyl, indazolyl, 1-benzothienyl, 1-benzofuranyl, isoquinolinyl, 5,6,7,8-tetrahydroquinolinyl, 3,4-dihydro-2H-chromenyl and 3,4-dihydro-2H-pyrido[3,2-b][1,4]oxazinyl, each of which is optionally substituted with 1, 2 or 3 substituents each independently selected from the group consisting of halo, cyano,
C₁₋₃alkyl, mono-haloC₁₋₃alkyl, poly-halo-C₁₋₃alkyl, C₁₋₃alkyloxy, C₃₋₆cycloalkyl, tetrahydro-2H-pyranyl, phenyl optionally substituted with C₁₋₃alkyl, and —NR^{1c}R^{1d}; wherein
R^{1c} is hydrogen or C₁₋₃alkyl, R^{1d} is C₁₋₃alkyl, or NR^{1c}R^{1d} form together 1-pyrrolidinyl, 1-piperidinyl, 4-piperazinyl, 4-morpholinyl or 1H-imidazolyl, each of which is optionally substituted with C₁₋₃alkyl; and
R² is hydrogen or C₁₋₃alkyl;
or a pharmaceutically acceptable addition salt or a solvate thereof.

2. The compound according to claim 1, wherein
X is S or O;
R is phenyl optionally substituted with 1, 2 or 3 substituents each independently selected from the group consisting of halo; hydroxyl; C₁₋₃alkyl; cyano; nitro; Het; Ar; (C₁₋₃alkyloxy)C₁₋₃alkyl-NH—C₁₋₃alkyl-; C₁₋₆alkyloxy optionally substituted with cyano, or C₁₋₃alkyloxy; C₂₋₆alkynyloxy; tetrahydro-2H-pyranyloxy; Ar-oxy-; Het-oxy-;
—NR^{a}R^{b}; a divalent —NH—CH₂CH₂—O— substituent optionally substituted with 1 or 2 substituents each independently selected from halo and oxo; and R³—C₁₋₆alkyloxy-; wherein
Het is selected from pyridinyl and pyrimidinyl, each of which can be optionally substituted with cyano;
Ar is phenyl;
R^{a} is selected from H, or C₁₋₃alkyl; and
R^{b} is selected from C₁₋₃alkyl, and (C₁₋₃alkyloxy)C₁₋₃alkyl(C═O)—;
R³ is selected from the group consisting of C₃₋₆cycloalkyl; Het¹; Ar¹; tetrahydro-2H-pyranyl; C₃₋₆cycloalkyloxy; tetrahydro-2H-pyranyloxy; Het¹-oxy-; and Ar¹-oxy-; wherein
Ar¹ is phenyl optionally substituted with halo, cyano, C₁₋₃alkyl, and C₁₋₃alkyloxy;
Het¹ is selected from the group consisting of pyridyl, pyrimidinyl, pyrazinyl, pyridazinyl, thienyl, pyrazolyl, isoxazolyl, 1H-imidazolyl, thiazolyl, oxazolyl, 1H-indolyl, and 1H-indazolyl; each of which is optionally substituted with 1 or 2 substituents each independently selected from the group consisting of halo, cyano, and C₁₋₃alkyl;
R¹ is selected from the group consisting of hydrogen; halo; cyano; C₁₋₃alkyl optionally substituted with hydroxyl or C₁₋₃alkyloxy; C₃₋₆cycloalkyl; C₁₋₃alkyloxy; C₁₋₃alkyloxy-(C═O)—; C₁₋₃alkyloxyC₂₋₃alkenyl; (halo-phenyl)-C₂₋₃alkenyl-; heterocyclyl; homoaryl; heteroaryl; homoaryl-CH₂-oxy; and heteroaryl-CH₂-oxy; wherein
heterocyclyl is 3,4-dihydro-2H-pyranyl;
homoaryl is phenyl optionally substituted with 1, 2 or 3 substituents each independently selected from the group consisting of halo, hydroxyl, cyano, C₁₋₃alkyl, mono-halo-C₁₋₃alkyl, poly-halo-C₁₋₃alkyl, cyano-C₁₋₃alkyl, C₁₋₃alkyloxy, mono-halo-C₁₋₃alkyloxy, poly-halo-C₁₋₃alkyloxy, C₁₋₃alkyloxy-(C═O)—, phenyloxy-, NR^{1a}R^{1b}, and
—(C═O)NR^{1a}R^{1b}; or is naphthalenyl, optionally substituted with C₁₋₃alkyl or
C₁₋₃alkyloxy; wherein R^{1a} is hydrogen or C₁₋₃alkyl and R^{1b} is C₁₋₃alkyl, or NR^{1a}R^{1b} form together a 4-morpholinyl;
heteroaryl is selected from the group consisting of pyridyl, 2-oxo-1,2-dihydropyridinyl, 6-oxo-1,6-dihydropyridinyl, pyrimidinyl, pyrazinyl, pyridazinyl, pyrazolyl, isoxazolyl, oxazolyl, thiophenyl, indolyl, indazolyl, 1-benzothienyl, 1-benzofuranyl, isoquinolinyl, 5,6,7,8-tetrahydroquinolinyl, 3,4-dihydro-2H-chromenyl and 3,4-dihydro-2H-pyrido[3,2-b][1,4]oxazinyl, each of which is optionally substituted with 1, 2 or 3 substituents each independently selected from the group consisting of halo, cyano,
C₁₋₃alkyl, mono-haloC₁₋₃alkyl, poly-halo-C₁₋₃alkyl, C₁₋₃alkyloxy, tetrahydro-2H-pyranyl, phenyl optionally substituted with C₁₋₃alkyl, and —NR^{1c}R^{1d}; wherein
R^{1c} is hydrogen or C₁₋₃alkyl, R^{1d} is C₁₋₃alkyl, or NR^{1c}R^{1d} form together 1-pyrrolidinyl, 1-piperidinyl, 4-piperazinyl, 4-morpholinyl or 1H-imidazolyl, each of which is optionally substituted with C₁₋₃alkyl; and
R² is hydrogen or C₁₋₃alkyl.

3. The compound according to claim 1, wherein
X is S or O;
R is phenyl optionally substituted with 1, 2 or 3 substituents each independently selected from the group consisting of halo; hydroxyl; nitro; Het; C₁₋₆alkyloxy optionally substituted with cyano, or C₁₋₃alkyloxy; C₂₋₆alkynyloxy; tetrahydro-2H-pyranyloxy; Het-oxy-; —NR^{a}R^{b}; a divalent —NH—CH₂CH₂—O— substituent optionally substituted with 1 or 2 substituents each independently selected from halo and oxo; and R³—C₁₋₆alkyloxy-; wherein
Het is selected from pyridinyl and pyrimidinyl, each of which can be optionally substituted with cyano;
Ar is phenyl;
R^{a} is H; and
R^{b} is (C₁₋₃alkyloxy)C₁₋₃alkyl(C═O)—;
R³ is selected from the group consisting of C₃₋₆cycloalkyl; Het¹; Ar¹; tetrahydro-2H-pyranyl; C₃₋₆cycloalkyloxy; tetrahydro-2H-pyranyloxy; Het¹-oxy-; and Ar¹-oxy-; wherein
Ar¹ is phenyl optionally substituted with halo, cyano, C₁₋₃alkyl, and C₁₋₃alkyloxy;
Het¹ is selected from the group consisting of pyridyl, pyrimidinyl, isoxazolyl, 1H-imidazolyl, thiazolyl, and 1H-indazolyl; each of which is optionally substituted with 1 or 2 substituents each independently selected from C₁₋₃alkyl;
R¹ is selected from the group consisting of hydrogen; halo; cyano; C₁₋₃alkyl optionally substituted with hydroxyl or C₁₋₃alkyloxy; C₃₋₆cycloalkyl; C₁₋₃alkyloxy; C₁₋₃alkyloxy-(C═O)—; C₁₋₃alkyloxyC₂₋₃alkenyl; (halo-phenyl)-C₂₋₃alkenyl-; heterocyclyl; homoaryl; heteroaryl; homoaryl-CH₂-oxy; and heteroaryl-CH₂-oxy; wherein
heterocyclyl is 3,4-dihydro-2H-pyranyl;
homoaryl is phenyl optionally substituted with 1, 2 or 3 substituents each independently selected from the group consisting of halo, hydroxyl, cyano, C₁₋₃alkyl, mono-halo-C₁₋₃alkyl, poly-halo-C₁₋₃alkyl, cyano-C₁₋₃alkyl, C₁₋₃alkyloxy, mono-halo-C₁₋₃alkyloxy, poly-halo-C₁₋₃alkyloxy, C₁₋₃alkyloxy-(C═O)—, phenyloxy-, NR^{1a}R^{1b}, and —(C═O)NR^{1a}R^{1b}; or is naphthalenyl, optionally substituted with C₁₋₃alkyl or C₁₋₃alkyloxy; wherein R^{1a} is hydrogen or C₁₋₃alkyl and R^{1b} is C₁₋₃alkyl, or NR^{1a}R^{1b} form together a 4-morpholinyl;
heteroaryl is selected from the group consisting of pyridyl, 2-oxo-1,2-dihydropyridinyl, 6-oxo-1,6-dihydropyridinyl, pyrimidinyl, pyrazinyl, pyridazinyl, pyrazolyl, isoxazolyl, oxazolyl, thiophenyl, indolyl, indazolyl, 1-benzothienyl, 1-benzofuranyl, isoquinolinyl, 5,6,7,8-tetrahydroquinolinyl, 3,4-dihydro-2H-chromenyl and 3,4-dihydro-2H-pyrido[3,2-b][1,4]oxazinyl, each of which is optionally substituted with 1, 2 or 3 substituents each independently selected from the group consisting of halo, cyano,
C₁₋₃alkyl, mono-haloC₁₋₃alkyl, poly-halo-C₁₋₃alkyl, C₁₋₃alkyloxy, tetrahydro-2H-pyranyl, phenyl optionally substituted with C₁₋₃alkyl, and —NR^{1c}R^{1d}; wherein
R^{1c} is hydrogen or C₁₋₃alkyl, R^{1d} is C₁₋₃alkyl, or NR^{1c}R^{1d} form together 1-pyrrolidinyl, 4-piperazinyl, or 1H-imidazolyl, each of which is optionally substituted with C₁₋₃alkyl; and
R² is hydrogen or C₁₋₃alkyl.

4. The compound according to claim 3, wherein
R is phenyl optionally substituted with 1, 2 or 3 substituents each independently selected from the group consisting of halo; and Het; wherein
Het is selected from pyridinyl and pyrimidinyl, each of which can be optionally substituted with cyano.

5. The compound according to claim 3, wherein
X is S or O;
R is phenyl optionally substituted with 1, 2 or 3 substituents each independently selected from the group consisting of halo; C₁₋₆alkyloxy optionally substituted with cyano, or C₁₋₃alkyloxy; C₂₋₆alkynyloxy; tetrahydro-2H-pyranyloxy; and R³—C₁₋₆alkyloxy-; wherein
R³ is selected from the group consisting of C₃₋₆cycloalkyl; Ar; tetrahydro-2H-pyranyl; C₃₋₆cycloalkyloxy; tetrahydro-2H-pyranyloxy; Het¹-oxy-; and Ar¹-oxy-; wherein
Ar¹ is phenyl optionally substituted with halo, cyano, C₁₋₃alkyl, and C₁₋₃alkyloxy;
Het¹ is selected from the group consisting of pyridyl, pyrimidinyl, isoxazolyl, 1H-imidazolyl, thiazolyl, and 1H-indazolyl; each of which is optionally substituted with 1 or 2 substituents each independently selected from C₁₋₃alkyl;
R¹ is selected from the group consisting of hydrogen; halo; homoaryl; and heteroaryl; wherein
homoaryl is phenyl optionally substituted with 1 or 2 or 3 substituents each independently selected from the group consisting of halo, cyano, C₁₋₃alkyl, and
C₁₋₃alkyloxy;
heteroaryl is selected from the group consisting of pyridyl and isoxazolyl, each of which is optionally substituted with 1 or 2 substituents each independently selected from the group consisting of halo, C₁₋₃alkyl and C₁₋₃alkyloxy; and
R² is hydrogen or C₁₋₃alkyl.

6. The compound according to claim 4, wherein
X is S or O;
R¹ is homoaryl or heteroaryl; wherein
homoaryl is phenyl optionally substituted with 1 or 2 substituents each independently selected from the group consisting of halo, cyano, and C₁₋₃alkyl;
heteroaryl is selected from the group consisting of pyridyl, and isoxazolyl, each of which is optionally substituted with 1 or 2 substituents each independently selected from the group consisting of halo, cyano, and C₁₋₃alkyl; and
R² is hydrogen or C₁₋₃alkyl.

7. A pharmaceutical composition comprising a therapeutically effective amount of a compound according to claim 1 and a pharmaceutically acceptable carrier.

8. A process for preparing a pharmaceutical composition comprising mixing a pharmaceutically acceptable carrier with a therapeutically effective amount of a compound according to claim 1.

9. (canceled)

10. (canceled)

11. A method of treating a disorder selected from the group consisting of Alzheimer's disease, mild cognitive impairment, senility, dementia, dementia with Lewy bodies, Down's syndrome, dementia associated with stroke, dementia associated with Parkinson's disease, and dementia associated with beta-amyloid comprising administering to a subject in need thereof, a therapeutically effective amount of a compound according to claim 1.

12. A method for modulating beta-site amyloid cleaving enzyme activity, comprising administering to a subject in need thereof, a therapeutically effective amount of a compound according to claim 1.

13. (canceled)

14. A process for the preparation of a compound according to Formula (I-a) or (I-b) wherein R, Het^{1a}, R^{1a} and R² are as defined in claim 1, comprising steps a) or b)
a) subjecting compound of Formula (XIV) to a Suzuki type reaction by heating with an appropriate boronic acid or ester in a suitable solvent, using an appropriate catalyst, in the presence of a suitable base 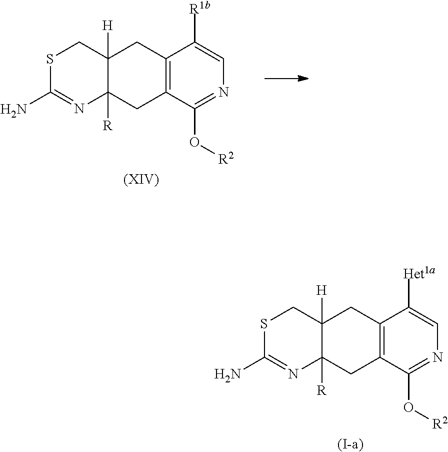
b) subjecting a compound of Formula (III-i), wherein R² is as defined in any one of claims 1 to 6, PG is a base labile protecting group and R is a phenyl having at least a substituent selected from hydroxyl, is first alkylated at the phenol with a suitable alkylating agent in the presence of a base and subsequently, a Suzuki reaction is performed using an appropriate base, such as potassium carbonate

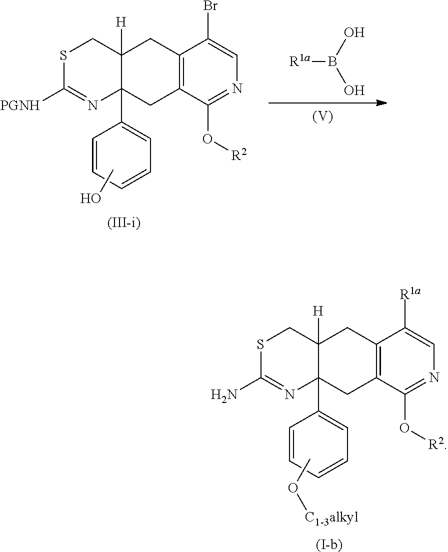

15. A compound of Formula (III-i′) 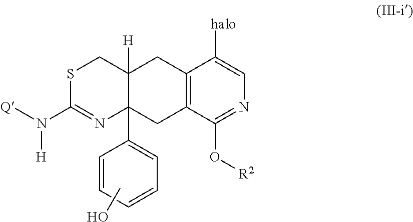
wherein Q′ is H or a protecting group, halo is bromo or chloro, in particular bromo, and R² is as defined in claim 1.

16. A method of treating a disorder selected from the group consisting of Alzheimer's disease, mild cognitive impairment, senility, dementia, dementia with Lewy bodies, Down's syndrome, dementia associated with stroke, dementia associated with Parkinson's disease, and dementia associated with beta-amyloid comprising administering to a subject in need thereof, a therapeutically effective amount of a pharmaceutical composition according to claim 7.

17. A method for modulating beta-site amyloid cleaving enzyme activity, comprising administering to a subject in need thereof, a therapeutically effective amount of a pharmaceutical composition according to claim 7.
